SEQUENCE LISTING (43 sequences)

SEQ ID NO: 1 - DNA, 14 bp, synthetic construct
  source 1..14: mol_type = other DNA
ggtgcatcgatgca

SEQ ID NO: 2 - DNA, 14 bp, synthetic construct
  source 1..14: mol_type = other DNA
ggtgcatgcatgca

SEQ ID NO: 3 - DNA, 14 bp, synthetic construct
  source 1..14: mol_type = other DNA
ggggtcaacgttga

SEQ ID NO: 4 - DNA, 14 bp, synthetic construct
  source 1..14: mol_type = other DNA
gggggacgatcgtc

SEQ ID NO: 5 - DNA, 15 bp, synthetic construct
  source 1..15: mol_type = other DNA
ggggacgacgtcgtg

SEQ ID NO: 6 - DNA, 10 bp, synthetic construct
  source 1..10: mol_type = other DNA
tcgtcgtttt

SEQ ID NO: 7 - DNA, 24 bp, synthetic construct
  source 1..24: mol_type = other DNA
tcgtcgttttgtcgttttgtcgtt

SEQ ID NO: 8 - DNA, 22 bp, synthetic construct
  source 1..22: mol_type = other DNA
tcgtcgttttcggcggccgccg

SEQ ID NO: 9 - DNA, 22 bp, synthetic construct
  source 1..22: mol_type = other DNA
tcgtcgttttcggcgcgcgccg

SEQ ID NO: 10 - DNA, 14 bp, synthetic construct
  source 1..14: mol_type = other DNA
  modified_base 1..3: mod_base = OTHER; note = phosphorothioated internucleotide linkage
ggtgcatcgatgca

SEQ ID NO: 11 - DNA, 14 bp, synthetic construct
  source 1..14: mol_type = other DNA
  modified_base 1..3: mod_base = OTHER; note = phosphorothioated internucleotide linkage
ggtgcatgcatgca

SEQ ID NO: 12 - DNA, 14 bp, synthetic construct
  source 1..14: mol_type = other DNA
  modified_base 1..3: mod_base = OTHER; note = phosphorothioated internucleotide linkage
ggggtcaacgttga

SEQ ID NO: 13 - DNA, 14 bp, synthetic construct
  source 1..14: mol_type = other DNA
  modified_base 1..3: mod_base = OTHER; note = phosphorothioated internucleotide linkage
gggggacgatcgtc

SEQ ID NO: 14 - DNA, 15 bp, synthetic construct
  source 1..15: mol_type = other DNA
  modified_base 1..4: mod_base = OTHER; note = phosphorothioated internucleotide linkage
ggggacgacgtcgtg

SEQ ID NO: 15 - DNA, 20 bp, synthetic construct
  source 1..20: mol_type = other DNA
ggtgcatcgatgcagggggg

SEQ ID NO: 16 - DNA, 20 bp, synthetic construct
  source 1..20: mol_type = other DNA
ggtgcatgcatgcagggggg

SEQ ID NO: 17 - DNA, 20 bp, synthetic construct
  source 1..20: mol_type = other DNA
tccatgacgttcctgatgct

SEQ ID NO: 18 - DNA, 20 bp, synthetic construct
  source 1..20: mol_type = other DNA
ggggtcaacgttgagggggg

SEQ ID NO: 19 - DNA, 20 bp, synthetic construct
  source 1..20: mol_type = other DNA
gggggacgatcgtcgggggg

SEQ ID NO: 20 - DNA, 21 bp, synthetic construct
  source 1..21: mol_type = other DNA
ggggacgacgtcgtggggggg

SEQ ID NO: 21 - DNA, 20 bp, synthetic construct
  source 1..20: mol_type = other DNA
tccatgacgttcctgatgct

SEQ ID NO: 22 - DNA, 20 bp, synthetic construct
  source 1..20: mol_type = other DNA
tccatgacgttcctgacgtt

SEQ ID NO: 23 - DNA, 24 bp, synthetic construct
  source 1..24: mol_type = other DNA
tcgtcgttttgtcgttttgtcgtt

SEQ ID NO: 24 - DNA, 22 bp, synthetic construct
  source 1..22: mol_type = other DNA
tcgtcgttgtcgttttgtcgtt

SEQ ID NO: 25 - DNA, 22 bp, synthetic construct
  source 1..22: mol_type = other DNA
tcgtcgttttcggcgcgcgccg

SEQ ID NO: 26 - DNA, 25 bp, synthetic construct
  source 1..25: mol_type = other DNA
tcgtcgtcgttcgaacgacgttgat

SEQ ID NO: 27 - DNA, 20 bp, synthetic construct
  source 1..20: mol_type = other DNA
  modified_base 16..20: mod_base = OTHER; note = phosphorothioated internucleotide linkage
  modified_base 1..3: mod_base = OTHER; note = phosphorothioated internucleotide linkage
ggtgcatcgatgcagggggg

SEQ ID NO: 28 - DNA, 20 bp, synthetic construct
  source 1..20: mol_type = other DNA
  modified_base 1..3: mod_base = OTHER; note = phosphorothioated internucleotide linkage
  modified_base 16..>20: mod_base = OTHER; note = phosphorothioated internucleotide linkage
ggtgcatcgatgcagggggg

SEQ ID NO: 29 - DNA, 20 bp, synthetic construct
  source 1..20: mol_type = other DNA
  modified_base 16..20: mod_base = OTHER; note = phosphorothioated internucleotide linkage
  modified_base 1..3: mod_base = OTHER; note = phosphorothioated internucleotide linkage
ggtgcatgcatgcagggggg

SEQ ID NO: 30 - DNA, 20 bp, synthetic construct
  source 1..20: mol_type = other DNA
  modified_base 1..20: mod_base = OTHER; note = phosphorothioated internucleotide linkage
ggtgcatcgatgcagggggg

SEQ ID NO: 31 - DNA, 20 bp, synthetic construct
  source 1..20: mol_type = other DNA
  modified_base 1..20: mod_base = OTHER; note = phosphorothioated internucleotide linkage
tccatgacgttcctgatgct

SEQ ID NO: 32 - DNA, 20 bp, synthetic construct
  source 1..20: mol_type = other DNA
  modified_base 1..3: mod_base = OTHER; note = phosphorothioated internucleotide linkage
  modified_base 15..20: mod_base = OTHER; note = phosphorothioated internucleotide linkage
ggggtcaacgttgagggggg

SEQ ID NO: 33 - DNA, 20 bp, synthetic construct
  source 1..20: mol_type = other DNA
  modified_base 15..20: mod_base = OTHER; note = phosphorothioated internucleotide linkage
  modified_base 1^2: mod_base = OTHER; note = phosphorothioated internucleotide linkage
ggggtcaacgttgagggggg

SEQ ID NO: 34 - DNA, 20 bp, synthetic construct
  source 1..20: mol_type = other DNA
  modified_base 1..3: mod_base = OTHER; note = phosphorothioated internucleotide linkage
  modified_base 16..20: mod_base = OTHER; note = phosphorothioated internucleotide linkage
gggggacgatcgtcgggggg

SEQ ID NO: 35 - DNA, 20 bp, synthetic construct
  source 1..20: mol_type = other DNA
  modified_base 1^2: mod_base = OTHER; note = phosphorothioated internucleotide linkage
  modified_base 15..20: mod_base = OTHER; note = phosphorothioated internucleotide linkage
gggggacgatcgtcgggggg

SEQ ID NO: 36 - DNA, 21 bp, synthetic construct
  source 1..21: mol_type = other DNA
  modified_base 1..4: mod_base = OTHER; note = phosphorothioated internucleotide linkage
  modified_base 16..21: mod_base = OTHER; note = phosphorothioated internucleotide linkage
ggggacgacgtcgtggggggg

SEQ ID NO: 37 - DNA, 21 bp, synthetic construct
  source 1..21: mol_type = other DNA
  modified_base 1..3: mod_base = OTHER; note = phosphorothioated internucleotide linkage
  modified_base 16..21: mod_base = OTHER; note = phosphorothioated internucleotide linkage
ggggacgacgtcgtggggggg

SEQ ID NO: 38 - DNA, 20 bp, synthetic construct
  source 1..20: mol_type = other DNA
  modified_base 1..20: mod_base = OTHER; note = phosphorothioated internucleotide linkage
tccatgacgttcctgatgct

SEQ ID NO: 39 - DNA, 20 bp, synthetic construct
  source 1..20: mol_type = other DNA
  modified_base 1..20: mod_base = OTHER; note = phosphorothioated internucleotide linkage
tccatgacgttcctgacgtt

SEQ ID NO: 40 - DNA, 24 bp, synthetic construct
  source 1..24: mol_type = other DNA
  modified_base 1..24: mod_base = OTHER; note = phosphorothioated internucleotide linkage
tcgtcgttttgtcgttttgtcgtt

SEQ ID NO: 41 - DNA, 22 bp, synthetic construct
  source 1..22: mol_type = other DNA
  modified_base 1..22: mod_base = OTHER; note = phosphorothioated internucleotide linkage
tcgtcgttgtcgttttgtcgtt

SEQ ID NO: 42 - DNA, 22 bp, synthetic construct
  source 1..22: mol_type = other DNA
  modified_base 1..22: mod_base = OTHER; note = phosphorothioated internucleotide linkage
tcgtcgttttcggcgcgcgccg

SEQ ID NO: 43 - DNA, 25 bp, synthetic construct
  source 1..25: mol_type = other DNA
  modified_base 1..25: mod_base = OTHER; note = phosphorothioated internucleotide linkage
tcgtcgtcgttcgaacgacgttgat

(19) United States
(12) Patent Application Publication
(10) Pub. No.: US 2025/0376689 A1
(43) Pub. Date: Dec. 11, 2025

(54) OLIGONUCLEOTIDES HAVING 6-THIO-2'-DEOXYGUANOSINE RESIDUES AND USES THEREOF

(71) Applicant: City of Hope, Duarte, CA (US)

(72) Inventors: Marcin Tomasz Kortylewski, Monrovia, CA (US); Chunsong Yu, Monrovia, CA (US); Piotr Marek Swiderski, San Dimas, CA (US)

(21) Appl. No.: 18/686,588

(22) PCT Filed: Aug. 31, 2022
(86) PCT No.: PCT/US2022/075745

§ 371 Date: Feb. 26, 2024

Related U.S. Application Data
(60) Provisional application No. 63/239,285, filed on Aug. 31, 2021.
(60) Provisional application No. 63/240,078, filed on Sep. 2, 2021.

(51) Int. Cl. C12N 15/117

(52) U.S. Cl. CPC C12N 15/117; C12N 2310/17; C12N 2310/321

(57) ABSTRACT

The disclosure provides, inter alia, oligonucleotides comprising 6-thio-2′-deoxy-guanosine residues. The oligonucleotides comprising said residues are used in pharmaceutical compositions and methods for treating cancer. The invention also includes CpG oligodeoxynucleotides in combination with the 6-thio-2-deoxy-guanosine residues in said pharmaceutical compositions and methods for treating cancer. Oligonucleotides of the invention may also include additional therapeutic moieties or exonuclease resistant moieties. The compositions and methods of the invention may employ additional pharmaceutical entities for the treatment of cancer.

### CROSS-REFERENCE TO RELATED APPLICATIONS

This application claims the benefit of priority to U.S. Application No. 63/240,078 filed Sep. 2, 2021, and U.S. Application No. 63/239,285 filed Aug. 31, 2021, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant no. CA213131 awarded by the National Institutes of Health. The government has certain rights in the invention.

### SEQUENCE LISTING

The material in the accompanying Sequence Listing is hereby incorporated by reference in its entirety. The accompanying file, named “048440-814001WO_SL_ST26.xml” was created on Aug. 30, 2022 and is 56,272 bytes. The file can be accessed using Microsoft Word on a computer that uses Windows OS.

### BACKGROUND

Telomerase is an enzyme expressed in about 90% of human cancers and critical for tumorigenesis and tumor survival. It is an attractive therapeutic target for small molecule inhibitors based on nucleoside analogs, such as 6-thio-2′-deoxyguanosine (6tdG). 6tdG can interrupt telomerase activity, thereby damaging/uncapping telomeres and inducing cancer cell senescence. A recent study suggested that telomere-associated DNA released from 6tdG treated cancer cells is also potently immunogenic and triggers immune responses through activation of cGAS/STING signaling in immune cells. However, small molecule inhibitors of telomerase can interfere with the activation and expansion of T cells, which depend on the telomerase activity (FIG. 14). Thus, systemic telomerase inhibition is likely to interfere with the generation of long-term antitumor immunity. Initial clinical testing (phase I/II) revealed toxicities related to the effect of telomerase inhibitors on hematopoietic stem cells, such as thrombocytopenia. Due to these limitations, telomere-targeted therapies have not moved past initial clinical trials. Thus, there is a need in the art to improve the safety and efficacy of 6tdG telomerase therapy. The disclosure is directed to this, as well as other, important end.

### BRIEF SUMMARY

Provided herein are 6tdG-oligonucleotides comprising at least one 6-thio-2′-deoxyguanosine residue. The 6tdG-oligonucleotides can comprise from 2 to 100 nucleotides. The 6tdG-oligonucleotides can comprise one or more modified nucleotides (e.g., modified bases, phosphorothioated internucleotide linkages). The 6tdG-oligonucleotides can be oligodeoxynucleotides, such as 6tdG-CpG oligodeoxynucleotides. The 6tdG-oligonucleotides can optionally include one or more therapeutic moieties.

Provided herein are methods of treating cancer in a patient by administering an effective amount of the 6tdG-oligonucleotides described herein (i.e., oligonucleotides comprising a 6-thio-2′-deoxyguanosine residue) or pharmaceutical compositions comprising the 6tdG-oligonucleotides described herein and a pharmaceutically acceptable excipient. In embodiments, the cancer expresses telomerase reverse transcriptase.

Provided herein are methods of treating cancer in a patient by administering an effective amount of a CpG oligonucleotide and an effective amount of 6-thio-2′-deoxyguanosine. In embodiments, the cancer expresses telomerase reverse transcriptase.

These and other embodiments of the disclosure are described in detail herein.

### BRIEF DESCRIPTION OF THE DRAWINGS

FIGS. 1A-1C show different fluorescent spectra of various CpG oligodeoxynucleotides (ODNs) used in the study. Unmodified CpG (D19), CpG(6tdG)₆-p, CpG(6tdG*)₆-p, CpG(6tdG)₅G, or CpG(6tdG)₅*G ODNs (FIG. 1A) were dissolved in nuclease-free water at 40 μM concentrations and analyzed using spectral scanning on the Cytation3 plate reader at 260 nm. FIG. 1B: Chemical modifications of the 3′ end of CpG(6tdG)6-p. FIG. 1C: The absorbance peak at 340 nm indicates detection of the incorporated of 6tdG phosphoramidites in ODNs.

FIGS. 2A-2H shows serum stability of CpG and 6tdG-modified ODN variants. CpG (D19), CpG(6tdG)₆-p, CpG(6tdG*)₆-p, CpG(6tdG)₅G, or CpG(6tdG)₅*G were incubated in 50% human serum at 37° C. for indicated times and then resolved by electrophoresis on the native 15% polyacrylamide gels. The band intensities were quantified, and the estimated oligonucleotide half-lives are shown.

FIGS. 3A-3H show the direct cytotoxic effects of 6tdG alone and CpG(6tdG)₅G on a variety of human cancer cell lines. (FIG. 3A) The effect of CpG(6tdG)₅G was assessed in vitro on a standard NCI-60 panel of human cancer cells with additional 6 pancreatic cell lines; shown are IC50 values calculated for the individual cell lines. Green: high sensitivity; yellow: moderate sensitivity; red—poor sensitivity to cytotoxic effects. (FIGS. 3B-3H) Comparison of dose-dependent toxicity of 6tdG small molecule vs. oligomeric CpG(6tdG)₅G. Human acute myeloid leukemia MOLM14 (FIG. 3B), HL-60 (FIG. 3C), K562 (FIG. 3D), OCI-Ly3 (FIG. 3E) OCI-Ly10 (FIG. 3F) diffuse large B cell lymphoma, LN-S17/TLR9 prostate cancer cells (FIG. 3G) and SCC1 head-neck cancer cells (FIG. 3H) were incubated in vitro in the presence of 6tdG alone or CpG(6tdG)₅G oligonucleotides. Cell viability was assessed using Cell Counting Kit-8 (CCK-8) viability assay and the half-maximal inhibitory doses (IC50) were calculated when possible; means±SEM (n=3).

FIGS. 4A-4C show that the 6tdG small molecule, but not the CpG-6tdG oligomers, is cytotoxic to activated T cells. (FIG. 4A) Neither 6tdG nor CpG-6tdG oligomers affect viability of resting human lymphocytes. PMBCs from 3 healthy donors were seeded at a density of 10⁷/mL and treated with CpG alone, 6tdG alone, combination of CpG and 6tdG, CpG(6tdG)₆-p, CpG(6tdG)₅G for 2 days. Cell apoptosis was measured using flow cytometry after staining for Annexin V and 7AAD. Representative results obtained from 3 independent experiments; means±SEM (n=3). (FIG. 4B) Human T cells isolated from 3 healthy donor PBMCs using negative selection were activated with CD3/CD28 beads in the presence of 20 ng/mL human IL-2 to induce proliferation. After activation, T cells were incubated in the presence of CpG alone, 6tdG alone, combination of CpG and 6tdG, CpG(6tdG)₆-p or CpG(6tdG)₅G for the indicated times before subject to viability analysis in flow cytometry. (FIG. 4C) Activated and proliferating T cells, but not naïve/resting T cells, have high levels of telomerase activity. The telomerase activity was assayed using TRAPeze kit (EMD Millipore), a telomerase repeated amplification protocol, and PCR products were qualitatively analyzed in a PAGE gel.

FIGS. 5A-5F show the cytotoxic effects of 6tdG alone and 6tdG-modified CpG ODN variants on telomerase-positive mouse prostate cancer cells. Mouse RM9 prostate cancer cells were incubated in vitro in the presence of 6tdG alone or CpG(6tdG)₆-p, CpG(6tdG*)₆-p and CpG(6tdG)₅G oligonucleotides. FIG. 5A-5D: Cell viability was assessed using cell counting kit-8 (CCK-8) viability assay and the half-maximal inhibitory doses (IC50) were calculated when possible. FIG. 5E: Representative microscopic images for the RM9 cells treated with various concentrations of 6tdG alone, CpG(6tdG)₅G or CpG(6tdG)₆-p. Scale bar=100 μm. FIG. 5F: RM9 cells show high levels of telomerase activity. The telomerase activity of RM9 was assayed by TRAPeze kit (EMD Millipore), a telomerase repeated amplification protocol, and PCR products were qualitatively analyzed in a PAGE gel.

FIGS. 6A-6H show that targeted delivery of 6tdG by CpG(6tdG)₆-p and CpG(6tdG)₅G ODNs induced apoptosis in prostate cancer cells. Mouse RM9 prostate cancer cells were incubated in the presence of 6tdG alone, CpG ODN alone, the combination thereof, CpG(6tdG)₆-p, CpG(6tdG)₅G or with inactivated CpG(6tdG*)₆-p oligonucleotides. Cell apoptosis were measured using flow cytometry after staining for Annexin V and 7AAD. Representative results are shown; means±SEM (n=3).

FIGS. 7A-7N demonstrate that CpG-6tdG oligomers have enhanced potency compared to small molecule 6tdG telomerase inhibitor against an array of mouse cancer cells. Tramp-C1 (FIGS. 7A-7D), Tramp-C2 prostate (FIGS. 7E-7F), MC-38 colon cancer (FIGS. 7G-7H), Hepa1-6 liver cancer (FIGS. 7I-7J), MB49 bladder cancer (FIGS. 7K-7L), Renca kidney cancer cells (FIGS. 7M-7N) were incubated in vitro in the presence of 6tdG alone or CpG(6tdG)₅G or other oligomer variants as indicated. Cell viability was assessed using Cell Counting Kit-8 (CCK-8) viability assay and the half-maximal inhibitory doses (IC50) were calculated when possible; means±SEM (n=3).

FIGS. 8A-8C show that the combination of CpG and 6tdG promoted cancer cell immunogenicity on co-cultured dendritic cells (DCs). FIG. 8A: RM9 prostate cancer cells were treated with a combination of 5 μM 6tdG and 1 μM CpG, 1 μM CpG(6tdG)₆-p or PBS overnight, washed and incubated for the additional 3 days in fresh media. The dying cancer cells were then harvested and co-cultured for 18 h with freshly isolated, mouse primary splenic DC at a ratio of 1:10. The percentages of activated DCs (CD11c+MHC II+/CD80+ or CD86+) were analyzed by flow cytometry. FIG. 8B: Bone-marrow derived dendritic cells (BMDCs) were co-cultured with cancer cells in the presence of CpG alone, 6tdG alone, combination of CpG and 6tdG, or CpG(6tdG)₆-p for 3 days. The expression of IFN-β mRNA was analyzed by real-time qPCR. Shown are results from three independent experiments; means±SEM (n=3); ****P<0.0001; *P<0.05 compared to the untreated group. FIG. 8C: TBK1 signaling downstream from STING is activated in primary mouse DCs co-cultured with RM9 cells pretreated using 6tdG alone, in combination with CpG or by CpG-(6tdG)₆-p oligomer. The protein content of dendritic cells was analyzed using western blot to evaluate levels of activated and total TBK1. The protein levels were normalized to beta-actin and quantified densitometrically.

FIGS. 9A-9D show that local administration of CpG(6tdG)₆-p conjugates improved antitumor efficacy against prostate tumors in vivo compared to the combination of unconjugated 6tdG and CpG. 2×10⁵. RM9 cells were implanted subcutaneously into the right flanks of C56BL/6 mice. When the tumor volume reached about 100 mm³, the mice were intratumorally injected with PBS, combination of equimolar amounts of CpG (2.5 mg/kg) and 6tdG (0.7 mg/kg) or CpG(6tdG)₆-p conjugate (2.5 mg/kg) every day for 6 days. The tumor volume was measured every day from Day 7 to Day 14, then mice were euthanized and tumors were analyzed using flow cytometry (A, B) CpG(6tdG)6-p conjugate reduced tumor growth (FIG. 9A) and tumor weight (FIG. 9B) significantly more effective than co-injection of CpG and 6tdG. FIG. 9C: The percentage of activated DCs (CD11c⁺MHC II⁺CD86⁺) in tumor draining lymph nodes (TDLN) was assessed by flow cytometry. FIG. 9D: The percentage of tumor-infiltrating CD8⁺ T cells was assessed using flow cytometry. Shown are results from three independent experiments; means±SEM (n=5); ****P<0.0001; ***P<0.001; *P<0.05; ns: no significance compared to the untreated group.

FIGS. 10A-10G show that systemic administration of CpG(6tdG)₆-p conjugate inhibited prostate tumors in mice. RM9 tumor-bearing immunocompetent C57BL/6 (FIG. 10A) or immunodeficient NSG (FIG. 10B) mice were injected intravenously using PBS, combination of equimolar amounts of CpG (5 mg/kg) and 6tdG (1.4 mg/kg) or CpG(6tdG)₆-p conjugate (5 mg/kg) every day for 6 days. In both models, CpG(6tdG)₆-p conjugate significantly reduced tumor growth more effectively than co-injection of CpG and 6tdG. FIGS. 10C-10G: The immunocompetent mice showed reduced levels of CD11b+Gr1+ MDSCs in various organs and increased percentages of activated DCs (CD11c⁺MHC II⁺CD86⁺) (FIG. 10F) and tumor-infiltrating CD8⁺ T cells (FIG. 10G) as assessed by flow cytometry. Results are shown as means±SEM (n=5); ****P<0.0001; ***P<0.001; *P<0.05; ns: not significant.

FIGS. 11A-11H show the combination of CpG and 6tdG resulted in abscopal effect against prostate tumors in mice. FIGS. 11A-11B: C57BL/6 mice were inoculated with RM9 tumor cells injected into two flanks to establish dual tumor model. Tumors on one side were injected peritumorally using PBS, combination of equimolar amounts of CpG (2.5 mg/kg) and 6tdG (0.7 mg/kg) or CpG(6tdG)₆-p conjugate (2.5 mg/kg) every day for 6 days. The treatment of CpG(6tdG)₆-p conjugate arrested tumor growth in both treated primary tumors (FIG. 11A) and the untreated distal tumors (FIG. 11B). The abscopal effects of CpG(6tdG)₆-p oligomer but not of the combination of CpG and 6tdG were associated with the increased ratio of CD8 T cells to Tregs in distal tumors (FIGS. 11C-11E) and tumor draining lymph nodes (FIG. 11F-11H) as assessed using flow cytometry. Results are shown as means±SEM (n=5); ****P<0.0001; ***P<0.001; *P<0.05; ns: not significant compared to the PBS control group.

FIGS. 12A-12C show that STAT3 silencing in RM9 cancer cells dramatically enhanced cytotoxic effects of CpG-6tdG conjugates. FIG. 12A: The decrease of STAT3 protein level in RM9 after treatment of STAT3 ASO in combination with CpG, CpG-(6tdG)₆-p, CpG-(6tdG)₅G. FIGS. 12B-12C: The frequency of viable RM9 cells after treatment of CpG, CpG-(6tdG)₆-p, CpG-(6tdG)₅G alone or with STAT3 ASO as assessed using flow cytometry after Annexin-V/77AAD staining.

FIGS. 13A-13E show that systemic administration of CpG(6tdG)₆-p conjugate reduced AML progression in syngeneic and in immunodeficient mice. FIGS. 13A-13D: 6tdG ODN results in regression of GFP-expressing Cbfb-Myh11-Mpl leukemia in C57BL/6j mice. Mice (n=5/group) were injected IV using 1.4 mg/kg 6tdG in combination with 5 mg/kg CpG or PBS alone for 3 consecutive days as indicated. FIG. 13A: Reduced percentages of GFP+ AML cells in the peripheral blood. FIG. 13B: Reduced splenomegaly in treated mice at the end of the study. FIG. 13C: Reduced frequency of GFP+ AML cells and increased percentage of CD8 T cells (FIG. 13D) in blood, spleen, and bone marrow at the end of the study. FIG. 13E: Immunodeficient NSG mice bearing mouse C1498 AML were treated using intravenous injections of PBS, combination of equimolar amounts of CpG (5 mg/kg) and 6tdG (1.4 mg/kg) or CpG(6tdG)₆-p conjugate (5 mg/kg) every day for 6 days. CpG(6tdG)₆-p conjugate significantly delayed leukemia progression.

FIG. 14 shows the putative mode of action of 6tdG oligonucleotide. The 6tdG ODN is internalized via scavenger receptor-mediated endocytosis by cancer cells as well as antigen-presenting immune cells (APCs) such as dendritic cells (DC) and macrophages (MAC). In cancer cells, 6tdG is cleaved by endosomal DNases. The released 6-thio-dG, disrupts telomerase activity, induces cancer cell death and release of damaged telomere DNA. The APCs recognize both 6tdG as well as telomere DNA, which synergistically induce immune signaling via TLR9 and/or cGAS/STING. Both pathways stimulate transcriptional regulation by interferon regulatory factors (IRFs) in the nucleus, promoting production of type I IFNs and antigen-presentation. As a result, APCs effectively stimulate expansion and antitumor activity of NK cells and T lymphocytes. 6tdG does not target T cells, which can preserve telomerase activity required for their proliferation and maximum antitumor activity.

FIGS. 15A-15B show that the cytotoxic effect of CpG-6tdG oligomers is selective to telomerase-positive target cells. FIG. 15A. Telomerase activity was assessed in untransformed/hTERT⁻ and transformed/hTERT⁺ primary human kidney epithelial cells (RPTEC) using a telomerase repeated amplification protocol (TRAPeze assay) with PCR products analyzed quantitatively on a PAGE gel. FIG. 15B. Cytotoxic effects of CpG-6tdGO are TERT-dependent; viability of primary and hTERT-expressing renal epithelial cells incubated for 72 h with CpG-6tdGO was measured by flow cytometry; means±SEM.

FIGS. 16A-16B show that CpG-6tdGO induced telomeric DNA damage in RM9 prostate cancer cells similarly to 6tdG nucleoside alone. FIG. 16A. CpG-6tdGO induced telomeric DNA damage in RM9 prostate cancer cells similar to 6tdG nucleoside. Fluorescent in situ hybridization results using telomere-specific probe (green) and γH2AX-specific antibodies (red), arrows indicate signal colocalization and telomere damage-induced foci. FIG. 16B. Bar graph with the quantification of FISH results; shown are telomere dysfunction-induced foci (TIFs) per nucleus, means±SEM (n=20).

FIGS. 17A-17E show the cell-selective internalization and endocytosis of CpG-6tdGO by cancer cells and immune cells. FIGS. 17A-17B. Dose-dependent cell-selective uptake of fluorescently-labeled CpG-6tdGO^{Cy3} by (FIG. 17A) human OCI-Ly3 and OCI-ly18 diffuse large B-cell lymphoma cells, and by (FIG. 17B) mouse RM9, human LNS17TTLR9 and DU145 prostate cancer cells at 2 h measured cytofluorimetrically. FIG. 17C. CpG-6tdGO^{Cy3} uptake by primary human immune cells, such as CD303⁺ plasmacytoid dendritic cells, CD19⁺ B cells, and CD3⁺ T cells. FIG. 17D. CpG-6tdGO is internalized by an active, scavenger receptor-mediated endocytic uptake. Normalized oligonucleotide uptake was assessed in RM9 cells pretreated with endocytosis inhibitors or cultured at 4° C.; means±SEM. FIG. 17E. Intracellular localization of CpG-6tdGO^{Cy3} in RM9 cells; a representative confocal microscopy image.

FIG. 18 shows that intravenously injected CpG-6tdGO preferentially accumulated in cancer cells and in tumor-associated myeloid cells. In vivo biodistribution of IV injected CpG-6tdGO was measure in RM9-luc/mCherry tumor-bearing mice injected IV once with 5 mg/kg Cy3-labeled CpG-6tdGO and euthanized at 3 and 18 h to harvest various organs. The percentages of Cy3⁺ cells within subcutaneously grown tumors were assessed using flow cytometry; mCherry⁺ cancer cells, CD11c⁺ DCs, CD11b⁺Gr1⁺MDSCs, and CD3⁺ T cells; means±SEM.

FIGS. 19A-19D show that systemic administration of CpG-6tdGO arrested acute myeloid leukemia (AML) progression and activated immune responses in mice. Mice bearing CMM (Cbfb-MYH11/Mpl) AML were treated from day 6 with 2.5 mg/kg CpG-6tdGO or an equimolar amount of 6tdG nucleoside and CpG alone daily for total of six times or left untreated. FIG. 19A. The percentages of GFP⁺ CMM cells in blood were assess every other day to monitor leukemia progression; means±SEM (n=5). FIG. 19B. Reduced splenomegaly in CpG-6tdGO-treated mice. Spleens were harvested, photographed and weighted at the end of study. FIG. 19C. CpG-6tdGO extends survival of CMM-bearing mice. Results from an independent study following the same treatment dosing and schedule (n=10). FIG. 19D. Frequencies of leukemia stem cells (c-kit+lin−), differentiated AML cells (MHCII⁺CD80⁺GFP⁺), and T cells (CD3⁺) in bone marrow of CMM-bearing mice were analyzed using flow cytometry; means±SEM (n=5).

FIGS. 20A-20F show that systemic administration of CpG-6tdGO induced regression of patient-derived AML xenograft in mice. FIG. 20A. Mice bearing patient sample-derived AML were treated using 5 mg/kg CpG-6tdGO or control PBS daily for total 6 times; means±SEM (n=6). The level of hCD45⁺mCD45⁻ was assessed every other day to monitor the progression of leukemia. FIG. 20B. CpG-6tdGO reduced splenomegaly in AML-bearing mice; representative photographs (left) and quantification of organ weights (right). FIGS. 20C-20F. At the end of study, the percentages of hCD45⁺FmCD45⁻ cells (FIG. 20C), AML cell viability (FIG. 20D), leukemia cell antigen presentation markers (FIG. 20E) and differentiation markers (FIG. 20F) in various organs were quantified using flow cytometry; means±SEM (n=6).

FIG. 21 shows that local intratumoral CpG-6tdGO injections arrested growth of human diffuse large B-cell lymphoma. OCI-Ly3-bearing NSG mice with tumor size around 100 mm³ were injected intratumorally with 2.5 mg/kg CpG-6tdGO or equimolar amounts of 6tdG and CpG every other day for six injections or only with PBS; shown are means±SEM (n=5).

FIGS. 22A-22C provide a safety assessment of CpG-6tdGO in humanized mice. HuCD34-NCG mice were humanized using CD34⁺ cord blood cells from two healthy donors, the successful engraftment of human immune cells was verified at week 14. Mice were injected IV using 5 mg/kg CpG-6tdGO or PBS every other day for two weeks and mice body weight was followed for an additional week (FIG. 22A). Later, mice were euthanized to harvest various organs to assess the effect of CpG-6tdGO. The percentages of total human hCD45⁺ immune cells (FIG. 22B), hematopoietic stem/progenitor cells (HSC/HPCs), myeloid cells, B cells and T cells (FIG. 22C) was measured using flow cytometry; means±SD (n=4). No negative effects on HSC/HPC, myeloid cells or T cells were observed.

### DETAILED DESCRIPTION

### Definitions

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The symbol “ [symbol] ” or denotes the point of attachment of a chemical moiety to the remainder of a molecule or chemical formula.

The term “6-thio-2′-deoxyguanosine” refers to a compound having the structure:

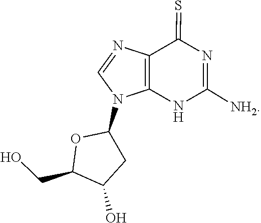 [SMILES: Nc1nc(=S)c2ncn([C@H]3C[C@H](O)[C@@H](CO)O3)c2[nH]1]

6-thio-2′-deoxyguanosine is a modified purine nucleoside analogue that is preferentially incorporated into telomeres in telomerase-positive cells, leading to telomere uncapping, genomic instability, and cell death, with minimal cytotoxic effects on telomerase-negative normal cells.

The term “6tdG residue” or “6-thio-2′-deoxyguanosine residue” refers to a nucleotide having the structure:

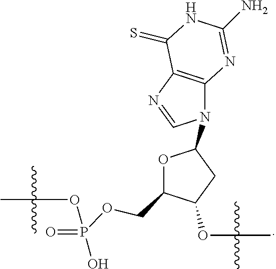 [SMILES: *O[C@H]1C[C@H](n2cnc3c(=S)[nH]c(N)nc32)O[C@@H]1COP(=O)(O)O*]

When the 6tdG residue is at the terminal position in a nucleic acid, then the 6tdG residue has an end-cap group (e.g., hydrogen) at the indicated point of attachment (by the symbol [symbol] ). Thus, when the 6tdG residue is at the terminal position in a nucleic acid, then the 6tdG residue has an end-cap group (e.g., hydrogen) at the indicated at the phosphate group or 3′ oxygen.

The term “6-thio-2′-deoxyguanosine-5′-monophosphorothioate residue” or “6tdG-monophosphorothioate residue” or “6tdG-PS residue” refers to a nucleotide having the structure:

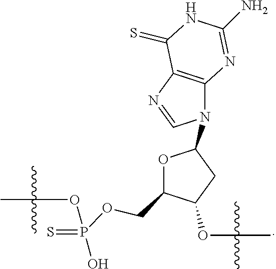 [SMILES: *O[C@H]1C[C@H](n2cnc3c(=S)[nH]c(N)nc32)O[C@@H]1COP(O)(=S)O*]

When the 6tdG-PS residue is at the terminal position in a nucleic acid, then the 6tdG-PS residue has an end-cap group at the indicated point of attachment (by the symbol [symbol] ).

The term “guanine residue” refers to a nucleotide having the structure:

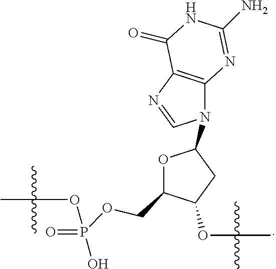 [SMILES: *O[C@H]1C[C@H](n2cnc3c(=O)[nH]c(N)nc32)O[C@@H]1COP(=O)(O)O*]

When the guanine residue is at the terminal position in a nucleic acid, then the guanine residue has an end-cap group at the point of attachment (by the symbol [symbol] ).

An “end-cap group” is used in accordance with its plain and ordinary meaning in the art. An end-cap group can be any known in the art, such as hydrogen or an exonuclease-resistant moiety. The end-cap group on the 5′ end (e.g., 5′ cap) and the 3′ end (e.g., 3′ cap) can be the same or different. In embodiments, an end-cap group is hydrogen. In embodiments, an end-cap group is an exonuclease-resistant moiety.

The term “6tdG-oligonucleotide” refers to an oligonucleotide that contains at least one 6-thio-2′-deoxyguanosine residue.

The term “6tdG-oligodeoxynucleotide” refers to an oligodeoxynucleotide that contains at least one 6-thio-2′-deoxyguanosine residue.

The term “6tdG-CpG oligodeoxynucleotide” refers to a CpG oligodeoxynucleotide that contains at least one 6-thio-2′-deoxyguanosine residue.

The term “phosphorothioated oligonucleotide” refers to a nucleic acid sequence in which one, some, or all the internucleotide linkages constitute a phosphorothioate linkage. In embodiments, a phosphorothioated oligonucleotide is 5 to 50 bases long, single-stranded, and partly or completely phosphorothioated. In embodiments, the phosphorothioated oligonucleotide contains 1 to 28 phosphorothioate internucleotide linkages. In embodiments, the phosphorothioated oligonucleotide contains 1 to 10 phosphorothioate internucleotide linkages. In embodiments, the phosphorothioated oligonucleotide is a phosphorothioated 6tdG-oligonucleotide. In embodiments, the phosphorothioated oligonucleotide is a phosphorothioated 6tdG-CpG oligonucleotide.

The term “phosphorothioated oligodeoxynucleotide” refers to a nucleic acid sequence in which one, some, or all the internucleotide linkages constitute a phosphorothioate linkage. In embodiments, phosphorothioated oligodeoxynucleotide (ODN) is 5 to 30 bases long, single-stranded, partly or completely phosphorothioated. In embodiments, the phosphorothioated ODN contains 1 to 28 phosphorothioate internucleotide linkages. In embodiments, the phosphorothioated oligodeoxynucleotide is a phosphorothioated 6tdG-oligodeoxynucleotide. In embodiments, the phosphorothioated oligodeoxynucleotide is a phosphorothioated 6tdG-CpG oligodeoxynucleotide.

The term “phosphorothioated internucleotide linkage” refers to a phosphorothioate bond between two adjacent nucleotide residues that replaces the natural phosphate internucleotide bond.

The term “6tdG mixmer” or “6-thio-2′-deoxyguanosine mixmer” refers to one or more 6tdG residues interspaced with adenine residues and/or thymine residues. In embodiments, the 6tdG residues are interspaced with: (a) 1 to 12 adenine residues; (b) 1 to 12 thymine residues; or (c) from 1 to about 12 adenine residues and from 1 to about 12 thymine residue. When the nucleic acid contains both adenine residues and thymine residues, the adenine residues and thymine residues can be random, block, or alternating. A 6tdG mixmer can alternatively be described, as an example, by the structure -(6tdG)z-(MIX)-, where z is an integer from 1 to 10, and (MIX) refers to 1 to 12 adenine residues, 1 to 12 thymine residues; or from 1 to about 12 adenine residues and from 1 to about 12 thymine residue (e.g., random, block, alternating). The (6tdG) and (MIX) groups can be repeated one or more times. For the purposes of illustration only, an example of a mixmer is -(6tdG)₄-AAA-(6tdG)₂-TTATA-(6tdG)₃-. In embodiments, a 6tdG residue can be a 6tdG-PS residue. In embodiments, the 6tdG residue can be replaced by a guanine residue, provided that there is at least one 6tdG residue present. In embodiments, a 6tdG mixmer comprises from 2 to 40 amino acid residues. In embodiments, a 6tdG mixmer comprises from 2 to 35 amino acid residues. In embodiments, a 6tdG mixmer comprises from 2 to 30 amino acid residues. In embodiments, a 6tdG mixmer comprises from 2 to 25 amino acid residues. In embodiments, a 6tdG mixmer comprises from 2 to 20 amino acid residues. In embodiments, a 6tdG mixmer comprises from 2 to 15 amino acid residues. In embodiments, a 6tdG mixmer comprises from 2 to 10 amino acid residues.

The term “exonuclease resistant moiety” refers to a compound (e.g., modified nucleotide, biotin, polyethylene glycol) that is resistant to nuclease degradation. In embodiments, a exonuclease resistant moiety is linked to the 3′ end of a nucleic acid to inhibit nuclease degradation.

The term “modified nucleotide” refers to a nucleotide that is modified from its natural state. The modification to the nucleotide can be to the base, the sugar, the phosphate, or two or more thereof. Nucleotides can be modified to include a hydroxyalkyl-terminated phosphate group, 2′-O-aminopropyl group, a 2′-constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, a locked nucleic acid (LNA) modification (e.g., the ribose is modified with an extra bridge connecting the 2′ oxygen and 4′ carbon), a dideoxy modification (e.g., the 2′ and 3′ positions on the ribose lack hydroxyl groups), an inverted deoxybasic modification. These modifications confer exonuclease resistance to the nucleotide.

The term “hydroxyalkyl-terminated phosphate group” refers to a phosphate group linked to a hydroxyalkyl group. In embodiments, a hydroxyalkyl-terminated phosphate group refers to a compound having the structure —[OP(═O)(OH)]_{w}—O—(CH₂)ₓOH, where w is 1, 2, or 3, and x is an integer from 1 to 20. In embodiments, w is 1. In embodiments, w is 2. In embodiments, w is 3. In embodiments, a hydroxyalkyl-terminated phosphate group refers to a compound having the structure —OP(═O)(OH)—O—(CH₂)ₓOH, where x is an integer from 1 to 20, which can alternatively be represented by the structure:

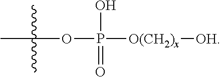

A “therapeutic agent” as used herein refers to a compound (e.g., nucleic acid, small molecule, DNA aptamer, RNA aptamer, or pharmaceutical composition described herein) that when administered to a subject will have the intended prophylactic effect, e.g., preventing or delaying the onset (or reoccurrence) of a disease, pathology or condition, or reducing the likelihood of the onset (or reoccurrence) of a disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the pathology or condition more tolerable to the patient; slowing in the rate of degeneration or decline; making the final point of degeneration less debilitating; or improving a patient's physical well-being. In embodiments, the therapeutic agent is an anti-cancer agent. In embodiments, the methods of treating cancer described herein further comprise administering to a patient an effective amount of an anti-cancer agent.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” or the like refer, in the usual and customary sense, to a linear sequence of nucleotides. The “oligonucleotide” can be a 6tdG-oligonucleotide, a 6tdG-oligodeoxynucleotide, a 6tdG-CpG oligodeoxynucleotide, a CpG oligodeoxynucleotide, and the like. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known modified nucleotides (e.g., nucleotide analogs) or modified internucleotide linkages (e.g., modified phosphate moieties, which are synthetic, naturally occurring, or non-naturally occurring. The modified nucleic acids may have similar binding properties as the reference nucleic acid, and which are metabolized in a manner similar to the reference nucleotides. Examples of modified internucleotide linkages include phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, an O-methylphosphoroamidite linkages (see Eckstein, Oligonucleotides and Analogues: A Practical Approach, Oxford University Press). Examples of modified nucleotide bases include 5-methyl cytidine and pseudouridine. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “residue” or “nucleotide residue” refers to a nucleotide having a base, sugar, and phosphate group. In context, it will be appreciated that the term “residue” can refer to a monovalent nucleotide residue or a divalent nucleotide residue that can be incorporated into a oligonucleotide.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (e.g., Fundamental Immunology (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (e.g., McCafferty et al., (1990) Nature 348:552). The term “antibody” also includes bivalent or bispecific molecules, diabodies, triabodies, and tetrabodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “aptamer” refers to single-stranded DNA or single-stranded RNA that can selectively bind to a specific target, including proteins, peptides, carbohydrates, small molecules, toxins, and live cells. Aptamers assume a variety of shapes due to their tendency to form helices and single-stranded loops. They are extremely versatile and bind targets with high selectivity and specificity. The term “aptamer” encompasses optimer ligands, split aptamers, X-aptamers, and the like.

The phrase “specifically (or selectively) binds” to an antibody or “specifically (or selectively) immunoreactive with,” when referring to a protein or peptide (e.g., binding of CpG to TLR9), refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and known in the art.

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “Toll-like receptor 9” or “TLR9” refers to any of the recombinant or naturally-occurring forms of the TLR9 protein or variants or homologs thereof that maintain TLR9 protein activity (e.g. within at least 50%, 80%, 90%, 95%, 96%, 97%, 98%, 99% or 100% activity compared to the TLR9 receptor). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TLR9 receptor polypeptide. In embodiments, the TLR9 receptor protein is substantially identical to or identical to the protein identified by UniProtKB reference number Q9NR96, or a variant or homolog having substantial identity thereto.

A “toll-like receptor 9-binding nucleic acid” refers to a nucleic acid capable of binding to toll like receptor 9. Exemplary nucleic acids include CpG oligodeoxynucleotides (ODN).

The term “CpG motif” is used in accordance with its plain and ordinary meaning and refers to at least one cytosine-guanine dinucleotide in an oligonucleotide or oligodeoxynucleotide. In embodiments, the “CpG motif” refers to at least one unmethylated cytosine-guanine dinucleotide in an oligonucleotide or oligodeoxynucleotide. In embodiments, “CpG motif” refers to at least one cytosine deoxynucleotide and guanine deoxynucleotide present in an oligodeoxynucleotide. In embodiments, the cytosine and guanine are connected through a phosphodiester internucleotide linkage or a phosphodiester derivative internucleotide linkage. In embodiments, the cytosine and guanine are connected through a phosphorothioate internucleotide linkage. In embodiments, the cytosine and guanine are connected through a phosphorothioate internucleotide linkage. In embodiments,

The term “CpG oligodeoxynucleotide” or “CpG ODN” refers to an oligonucleotide which contains at least one CpG motif and at least one nucleotide containing a deoxyribose. In embodiments, the CpG oligodeoxynucleotide comprises a phosphodiester internucleotide linkage, a phosphodiester derivative internucleotide linkage, or a combination thereof. In embodiments, a CpG ODN includes a phosphodiester internucleotide linkage. In embodiments, a CpG ODN includes a phosphodiester derivative internucleotide linkage. In embodiments, a CpG ODN includes a phosphorothioate internucleotide linkage. As defined herein, a “CpG oligodeoxynucleotide” does not contain a 6-thio-2′-deoxyguanosine residue (e.g., whereas a 6tdG-CpG oligodeoxynucleotide does contain a 6-thio-2′-deoxyguanosine residue).

The term “Class A CpG ODN” or “A-class CpG ODN” or “D-type CpG ODN” or “Class A CpG DNA sequence” refers to a CpG motif including oligodeoxynucleotide including one or more of poly-G sequence at the 5′, 3′, or both ends; an internal palindrome sequence including CpG motif; or one or more phosphodiester derivatives linking deoxynucleotides. In embodiments, a Class A CpG ODN includes poly-G sequence at the 5′, 3′, or both ends; an internal palindrome sequence including CpG motif; and one or more phosphodiester derivatives linking deoxynucleotides. In embodiments, the phosphodiester derivative is phosphorothioate Examples of Class A CpG ODNs include ODN D19, ODN 1585, ODN 2216, and ODN 2336, the sequences of which are known in the art.

The term “Class B CpG ODN” or “B-class CpG ODN” or “K-type CpG ODN” or “Class B CpG DNA sequence” refers to a CpG motif including oligodeoxynucleotide including one or more of a 6mer motif including a CpG motif: phosphodiester derivatives linking all deoxynucleotides. In embodiments, a Class B CpG ODN includes one or more copies of a 6mer motif including a CpG motif and phosphodiester derivatives linking all deoxynucleotides. In embodiments, the phosphodiester derivative is phosphorothioate. In embodiments, a Class B CpG ODN includes one 6mer motif including a CpG motif. In embodiments, a Class B CpG ODN includes two copies of a 6mer motif including a CpG motif. In embodiments, a Class B CpG ODN includes three copies of a 6mer motif including a CpG motif. In embodiments, a Class B CpG ODN includes four copies of a 6mer motif including a CpG motif. Examples of Class B CpG ODNs include ODN 1668, ODN 1826, ODN 2006, ODN 2007, ODN BW006, and ODN D-SL01, the sequences of which are known in the art.

The term “Class C CpG ODN” or “C-class CpG ODN” or “C-type CpG DNA sequence” refers to an oligodeoxynucleotide including a palindrome sequence including a CpG motif and phosphodiester derivatives (phosphorothioate) linking all deoxynucleotides. Examples of Class C CpG ODNs include ODN 2395, ODN M362, and ODN D-SL03, the sequences of which are known in the art.

The term “STAT” or “STAT transcription factor” are used interchangeably and refer to a “signal transducer and activator of transcription” protein and homologs thereof (e.g. STAT1, STAT2, STAT3, STAT4, STAT5A, STAT5B, STAT6, STAT7, STAT8, STAT7/8, STAT9). In aspects, “STAT transcription factor” refers to a human protein. Included in the term “STAT transcription factor” are the wildtype and mutant forms of the protein. In aspects, “STAT transcription factor” refers to the wildtype protein. In aspects, “STAT transcription factor” refers to a mutant protein. “Phosphorylated STAT” refers to a STAT protein that is phosphorylated and activated by the phosphorylation. In aspects, activation of a STAT transcription factor means the STAT is capable of activating transcription.

A “STAT3” or “STAT3 protein” refers to any of the recombinant or naturally-occurring forms of the Signal transducer and activator of transcription 3 (STAT3) protein or variants or homologs thereof that maintain STAT3 protein activity (e.g. within at least 50%, 80%, 90%, 95%, 96%, 97%, 98%, 99% or 100% activity compared to STAT3). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring STAT3 polypeptide. In aspects, the STAT3 protein is substantially identical to the protein identified by the NCBI reference number GI: 47458820, or a variant or homolog having substantial identity thereto. In aspects, the STAT3 protein is substantially identical to the protein identified by the NCBI reference number GI: 1610577068, or a variant or homolog having substantial identity thereto. In aspects, the STAT3 protein is substantially identical to the protein identified by the NCBI reference number GI: 1610577050, or a variant or homolog having substantial identity thereto. “Phosphorylated STAT3” refers to a STAT3 protein that is phosphorylated and activated by the phosphorylation. In aspects, a phosphorylated STAT3 is phosphorylated on tyrosine 705 or the residue corresponding to tyrosine 705 in homologs. In aspects, activation of STAT3 means the STAT3 is capable of activating transcription.

The terms “STAT3 gene” or “STAT3 sequence” as used herein refer to the genetically engineered gene or variants thereof that code for an STAT3 polypeptide capable of maintaining the activity of the STAT3 polypeptide (e.g., within at least 50%, 80%, 90%, 95%, 96%, 97%, 98%, 99% or 100% activity compared to the STAT3 polypeptide). In aspects, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to the STAT3 sequence. In aspects, STAT3 is substantially identical to the nucleic acid sequence identified by Accession No. NG_007370 or a variant or homolog having substantial identity thereto.

The term “STAT inhibitor” refers to any compound (e.g., small molecule, protein, nucleic acid sequence) capable of inhibiting STAT (e.g. STAT1, STAT2, STAT3, STAT4, STAT5A, STAT5B, STAT6, STAT7, STAT8, STAT7/8, STAT9). Exemplary STAT inhibitors include OPB-111077, AS-1517499, cryptotanshinone, galiellactone, LLL3, LLL12, niclosamide, pimozide, SH-4-54, STA-21, SPI, S31-201, X188-9, (E)-N′-((4-oxo-4H-chromen-3-yl)methylene)-nicotinohydrazide, XZH-5, SF-1066, SF-1087, H-Pro-Tyr-(PO₃H₂)-Leu-Lys-Thr-Lys-Ala-Ala-Val-Leu-Leu-Pro-Val-Leu-Leu-Ala-Ala-Pro-OH, and the like. In embodiments, the STAT inhibitor is a STAT3 inhibitor. In embodiments, the STAT inhibitor is a STAT-inhibiting nucleic acid sequence. The term “STAT inhibitor moiety” refers to a monovalent STAT inhibitor that can be covalently bonded to another compound (e.g., to a CpG oligonucleotide or a 6tdG-oligonucleotide).

The term “STAT-inhibiting nucleic acid sequence” refers to a nucleic acid sequence capable of inhibiting STAT (e.g. STAT1, STAT2, STAT3, STAT4, STAT5A, STAT5B, STAT6, STAT7, STAT8, STAT7/8, STAT9). In aspects the STAT-inhibiting nucleic acid sequence is a STAT3-inhibiting nucleic acid sequence.

“Contacting” is used in accordance with its plain ordinary meaning and refers to the process of allowing at least two distinct species (e.g. chemical compounds including biomolecules or cells) to become sufficiently proximal to react, interact, or physically touch. It should be appreciated; however, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a nucleic acid as described herein and a cell, protein, or enzyme.

“Control” or “control experiment” is used in accordance with its plain ordinary meaning and refers to an experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity of a protein in the absence of an olignucleotide (e.g., 6tdG-oligonucleotide) as described herein (including embodiments and examples). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 2-fold, 4-fold, 5-fold, 10-fold, or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In embodiments, expression or activity is 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

6tdG-Oligonucleotides

The disclosure provides 6tdG-oligonucleotides comprising at least one 6-thio-2′-deoxyguanosine residue. In embodiments, the 6tdG-oligonucleotide comprises two 6-thio-2′-deoxyguanosine residue. In embodiments, the 6tdG-oligonucleotide comprises at least two 6-thio-2′-deoxyguanosine residues. In embodiments, the 6tdG-oligonucleotide comprises at least three 6-thio-2′-deoxyguanosine residues. In embodiments, the 6tdG-oligonucleotide comprises at least four 6-thio-2′-deoxyguanosine residues. In embodiments, the 6tdG-oligonucleotide comprises at least five 6-thio-2′-deoxyguanosine residues. In embodiments, the 6tdG-oligonucleotide comprises at least six 6-thio-2′-deoxyguanosine residues. In embodiments, the 6tdG-oligonucleotide comprises from two to about forty 6-thio-2′-deoxyguanosine residues. In embodiments, the 6tdG-oligonucleotide comprises from two to about thirty 6-thio-2′-deoxyguanosine residues. In embodiments, the 6tdG-oligonucleotide comprises from two to about twenty-five 6-thio-2′-deoxyguanosine residues. In embodiments, the 6tdG-oligonucleotide comprises from two to about twenty 6-thio-2′-deoxyguanosine residues. In embodiments, the 6tdG-oligonucleotide comprises from two to about fifteen 6-thio-2′-deoxyguanosine residues. In embodiments, the 6tdG-oligonucleotide comprises from two to about ten 6-thio-2′-deoxyguanosine residues. In embodiments, the 6tdG-oligonucleotide comprises from three to six 6-thio-2′-deoxyguanosine residues. In embodiments, the 6tdG-oligonucleotide comprises from four to six 6-thio-2′-deoxyguanosine residues. In embodiments, the 6tdG-oligonucleotide further comprises a 6tdG mixmer.

In embodiments, the 6tdG-oligonucleotides comprise at least two contiguous (i.e., adjacent) 6-thio-2′-deoxyguanosine residues. In embodiments, the 6tdG-oligonucleotides comprise at least three contiguous (i.e., adjacent) 6-thio-2′-deoxyguanosine residues. In embodiments, the 6tdG-oligonucleotides comprise at least four contiguous (i.e., adjacent) 6-thio-2′-deoxyguanosine residues. In embodiments, the 6tdG-oligonucleotides comprise at least five contiguous (i.e., adjacent) 6-thio-2′-deoxyguanosine residues. In embodiments, the 6tdG-oligonucleotides comprise at least six contiguous (i.e., adjacent) 6-thio-2′-deoxyguanosine residues. In embodiments, the 6tdG-oligonucleotides comprise at least seven contiguous (i.e., adjacent) 6-thio-2′-deoxyguanosine residues. In embodiments, the 6tdG-oligonucleotides comprise at least eight contiguous (i.e., adjacent) 6-thio-2′-deoxyguanosine residues. In embodiments, the 6tdG-oligonucleotides comprise at least nine contiguous (i.e., adjacent) 6-thio-2′-deoxyguanosine residues. In embodiments, the 6tdG-oligonucleotides comprise at least ten contiguous (i.e., adjacent) 6-thio-2′-deoxyguanosine residues. In embodiments, the 6tdG-oligonucleotides comprise from two to twelve contiguous (i.e., adjacent) 6-thio-2′-deoxyguanosine residues. In embodiments, the 6tdG-oligonucleotides comprise from two to ten contiguous (i.e., adjacent) 6-thio-2′-deoxyguanosine residues. In embodiments, the 6tdG-oligonucleotides comprise from three to ten contiguous (i.e., adjacent) 6-thio-2′-deoxyguanosine residues. In embodiments, the 6tdG-oligonucleotides comprise from three to eight contiguous (i.e., adjacent) 6-thio-2′-deoxyguanosine residues. In embodiments, the 6tdG-oligonucleotides comprise from four to eight contiguous (i.e., adjacent) 6-thio-2′-deoxyguanosine residues. In embodiments, the 6tdG-oligonucleotides comprise from four to six contiguous (i.e., adjacent) 6-thio-2′-deoxyguanosine residues. In embodiments, the 6tdG-oligonucleotides comprise four contiguous (i.e., adjacent) 6-thio-2′-deoxyguanosine residues. In embodiments, the 6tdG-oligonucleotides comprise five contiguous (i.e., adjacent) 6-thio-2′-deoxyguanosine residues. In embodiments, the 6tdG-oligonucleotides comprise six contiguous (i.e., adjacent) 6-thio-2′-deoxyguanosine residues. In embodiments, the contiguous (i.e., adjacent) 6-thio-2′-deoxyguanosine residues are covalently bonded together via a phosphate internucleotide linkage or a modified phosphate internucleotide linkage. In embodiments, the contiguous (i.e., adjacent) 6-thio-2′-deoxyguanosine residues are covalently bonded together via a phosphate internucleotide linkage, a phosphorothioate internucleotide linkage, or a combination thereof. In embodiments, the contiguous (i.e., adjacent) 6-thio-2′-deoxyguanosine residues are covalently bonded together via a phosphate internucleotide linkage. In embodiments, the contiguous (i.e., adjacent) 6-thio-2′-deoxyguanosine residues are not covalently bonded together via a phosphorothioate internucleotide linkage. In embodiments, the contiguous (i.e., adjacent) 6-thio-2′-deoxyguanosine residues are not covalently bonded together via a modified internucleotide linkage.

In embodiments, the 6tdG-oligonucleotides further comprise one or more nucleotides or modified nucleotides (i.e., other than the 6-thio-2′-deoxyguanosine residue). In embodiments, the one or more nucleotides include an adenine residue, a deoxyadenine residue, a cytidine residue, a deoxycytidine residue, a uracil residue, or a deoxyuracil residue. In embodiments, the one or more nucleotides include an adenine residue, a cytidine residue, or a uracil residue. In embodiments, the one or more nucleotides include an adenine residue, a deoxyadenine residue, a guanine residue, a deoxyguanine residue, a thymine residue, a deoxythymine residue, a cytidine residue, a deoxycytidine residue, a uracil residue, or a deoxyuracil residue. In embodiments, the one or more nucleotides include an adenine residue, a deoxyadenine residue, a cytidine residue, a deoxycytidine residue, a uracil residue, a deoxyuracil residue, a modified adenine residue, a modified deoxyadenine residue, a modified cytidine residue, a modified deoxycytidine residue, a modified uracil residue, or a modified deoxyuracil residue. In embodiments, the one or more nucleotides include an adenine residue, a deoxyadenine residue, a guanine residue, a deoxyguanine residue, a thymine residue, a deoxythymine residue, a cytidine residue, a deoxycytidine residue, a uracil residue, a deoxyuracil residue, a modified adenine residue, a modified deoxyadenine residue, a modified guanine residue, a modified deoxyguanine residue, a modified thymine residue, a modified deoxythymine residue, a modified cytidine residue, a modified deoxycytidine residue, a modified uracil residue, and a modified deoxyuracil residue. In embodiments, the modified nucleotide residue comprises a modified base. In embodiments, the modified base comprises a dideoxy modification, an inverted deoxybasic modification, a 2′-O-aminopropyl group, a 2′ constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, or a locked nucleic acid modification. In embodiments, the modified nucleotide residue comprises a modified sugar. In embodiments, the modified nucleotide residue comprises a modified phosphate group (e.g., phosphorothioate group). In embodiments, the 6tdG-oligonucleotide further comprises a 6tdG mixmer.

In embodiments, the 6tdG-oligonucleotides described herein comprise from 2 to about 100 nucleotides. In embodiments, the 6tdG-oligonucleotides comprise from about 2 to about 50 nucleotides. In embodiments, the 6tdG-oligonucleotides comprise from about 2 to about 40 nucleotides. In embodiments, the 6tdG-oligonucleotides comprise from about 2 to about 35 nucleotides. In embodiments, the 6tdG-oligonucleotides comprise from about 2 to about 30 nucleotides. In embodiments, the 6tdG-oligonucleotides comprise from about 2 to about 25 nucleotides. In embodiments, the 6tdG-oligonucleotides comprise from about 10 to about 100 nucleotides. In embodiments, the 6tdG-oligonucleotides comprise from about 10 to about 50 nucleotides. In embodiments, the 6tdG-oligonucleotides comprise from about 10 to about 40 nucleotides. In embodiments, the 6tdG-oligonucleotides comprise from about 10 to about 35 nucleotides. In embodiments, the 6tdG-oligonucleotides comprise from about 10 to about 30 nucleotides. In embodiments, the 6tdG-oligonucleotides comprise from about 10 to about 25 nucleotides. In embodiments, the 6tdG-oligonucleotide comprises at least one phosphorthioate internucleotide linkage. In embodiments, the 6tdG-oligonucleotide is a oligodeoxynucleotide. In embodiments, the 6tdG-oligonucleotide is a oligodeoxynucleotide comprising at least one phosphorthioate internucleotide linkage.

In embodiments, the nucleosides in the 6tdG-oligonucleotide are covalently bonded together via a phosphodiester internucleotide linkage, a modified phosphodiester internucleotide linkage, or a combination thereof. In embodiments, the modified phosphodiester internucleotide linkage is a phosphorothioate internucleotide linkage, a phosphonoacetate internucleotide linkage, a methyl phosphonate internucleotide linkage, or a phosphonocarboxylate internucleotide linkage. In embodiments, the modified phosphodiester internucleotide linkage is a phosphorothioate internucleotide linkage. In embodiments, the 6tdG-oligonucleotide comprises at least one phosphorothioate internucleotide linkage. In embodiments, the 6tdG-oligonucleotide comprises at least two phosphorothioate internucleotide linkage. In embodiments, the 6tdG-oligonucleotide comprises at least three phosphorothioate internucleotide linkage. In embodiments, the 6tdG-oligonucleotide comprises at least one phosphorothioate internucleotide linkage, provided that the phosphorothioate internucleotide linkage is not between 6-thio-2′-deoxyguanosine residues. In embodiments, the 6tdG-oligonucleotide comprises at least two phosphorothioate internucleotide linkage, provided that the phosphorothioate internucleotide linkages are not between 6-thio-2′-deoxyguanosine residues. In embodiments, the 6tdG-oligonucleotide comprises at least three phosphorothioate internucleotide linkage, provided that the phosphorothioate internucleotide linkages are not between 6-thio-2′-deoxyguanosine residues.

In embodiments, the 6tdG-oligonucleotides described herein are phosphorothioated 6tdG-oligonucleotides. In embodiments, the 6tdG-oligonucleotides are oligodeoxynucleotides. In embodiments, the 6tdG-oligonucleotides are phosphorothioated oligodeoxynucleotides.

In embodiments, the disclosure provides 6tdG-oligonucleotides comprising: (i) at least two 6-thio-2′-deoxyguanosine residues; and (ii) at least one phosphorothioated internucleotide linkage. In embodiments, the disclosure provides 6tdG-oligonucleotides comprising: (i) at least two 6-thio-2′-deoxyguanosine residues; and (ii) at least one phosphorothioated internucleotide linkage, wherein the at least one phosphorothioated internucleotide linkage is not between the 6-thio-2′-deoxyguanosine residues. In embodiments, the disclosure provides 6tdG-oligonucleotides comprising: (i) at least two 6-thio-2′-deoxyguanosine residues; (ii) a nucleotide residue selected from the group consisting of an adenine residue, a deoxyadenine residue, a cytidine residue, a deoxycytidine residue, a uracil residue, or a deoxyuracil residue; and (iii) at least one phosphorothioated internucleotide linkage. In embodiments, the disclosure provides 6tdG-oligonucleotides comprising: (i) at least two 6-thio-2′-deoxyguanosine residues; (ii) a nucleotide residue selected from the group consisting of an adenine residue, a deoxyadenine residue, a cytidine residue, a deoxycytidine residue, a uracil residue, or a deoxyuracil residue; and (iii) at least one phosphorothioated internucleotide linkage, wherein the at least one phosphorothioated internucleotide linkage is not between the 6-thio-2′-deoxyguanosine residues.

In embodiments, the 6tdG-oligonucleotides described herein, including all embodiments thereof, further comprise a therapeutic moiety, an exonuclease resistant moiety, a detectable moiety, or a combination thereof. In embodiments, the therapeutic moiety, detectable moiety, or exonuclease resistant moiety are located at the 3′ end of the 6tdG-oligonucleotide. In embodiments, the therapeutic moiety, detectable moiety, or exonuclease resistant moiety are located at the 5′ end of the 6tdG-oligonucleotide. In embodiments, the therapeutic moiety, detectable moiety, or exonuclease resistant moiety are located at the 3′ end of the 6tdG-oligonucleotide and/or the 5′ end of the 6tdG-oligonucleotide.

In embodiments, the 6tdG-oligonucleotides further comprise a therapeutic moiety. The term “therapeutic moiety” refers to a monovalent therapeutic agent that is covalently bonded to an 6tdG-oligonucleotide described herein (including all embodiments thereof). In embodiments, the therapeutic moiety is a compound, such as small molecule, antibody, DNA aptamer, or RNA aptamer, that binds to a target molecule. In such an embodiment, the therapeutic moiety can be referred to as a “targeting moiety” because it binds to a target molecule. The target molecule can be a receptor (e.g., a cell surface receptor) or a protein or enzyme (e.g., a protein or enzyme that binds to a cell surface receptor). Thus, the therapeutic moiety (optionally referred to as a targeting moiety) can bind directly to a cell surface receptor or bind to a protein or enzyme in order to prevent that protein or enzyme from binding to a cell surface receptor or to interfere with a signaling pathway. In embodiments, the therapeutic moiety binds to a cell surface receptor to inhibit the activity of the cell surface receptor. In embodiments, the therapeutic moiety binds to a protein or enzyme to inhibit the ability of that protein or enzyme to bind to a cell surface receptor. In embodiments, the therapeutic moiety binds to a protein or enzyme to inhibit or otherwise interfere with a signaling pathway. The therapeutic moiety can be released from the 6tdG-oligonucleotide prior to binding to a target molecule or can remain bonded to the 6tdG-oligonucleotide when binding to the target molecule. Exemplary therapeutic moieties include monoclonal antibodies, DNA apatmers, RNA aptamers, folate, cholesterol, N-acetylgalactosamine, and STAT inhibitors. In embodiments, the monoclonal antibody is capable of binding to HER2, EFGR, VEGFR, HGFR, SLAMF7, GD2, CD19, CD20, CD30, CD33, CD38, CD70, PSCA, or PSMA. In embodiments, the therapeutic moiety is a nucleic acid. In embodiments, the therapeutic moiety is a small molecule. In embodiments, the therapeutic moiety is an antibody. In embodiments, the therapeutic moiety is a DNA aptamer. In embodiments, the therapeutic moiety is an RNA aptamer. In embodiments, the therapeutic moiety is a STAT inhibitor. In embodiments, the therapeutic moiety is a STAT3 inhibitor. In embodiments, the therapeutic moiety is at the 5′ end and/or the 3′ end of the 6tdG-oligonucleotide. In embodiments, the therapeutic moiety is at the 5′ end of the 6tdG-oligonucleotide. In embodiments, the therapeutic moiety is at the 3′ end of the 6tdG-oligonucleotide. In embodiments, a therapeutic moiety is at the 5′ end of the 6tdG-oligonucleotide and the 3′ end of the 6tdG-oligonucleotide, where the therapeutic moiety at the 3′ end and the 5′ end are the same or different.

In embodiments, the therapeutic moiety is an antibody or a fragment of an antibody. In embodiments, the therapeutic moiety is an antibody. In embodiments, the therapeutic moiety is a fragment of an antibody. In embodiments, the therapeutic moiety is a single chain antibody (scFv). In embodiments, the therapeutic moiety is a chimeric antibody. In embodiments, the therapeutic moiety is an antibody to HER2, EGFR, VEGFR, HGFR, SLAMF7, GD2, CD19, CD20, CD30, CD33, CD38, CD70, PSCA, or PSMA. In embodiments, the therapeutic moiety is an antibody to HER2. In embodiments, the therapeutic moiety is an antibody to EGFR. In embodiments, the therapeutic moiety is an antibody to VEGFR. In embodiments, the therapeutic moiety is an antibody to HGFR. In embodiments, the therapeutic moiety is an antibody to SLAMF7. In embodiments, the therapeutic moiety is an antibody to GD2. In embodiments, the therapeutic moiety is an antibody to CD19. In embodiments, the therapeutic moiety is an antibody to CD20. In embodiments, the therapeutic moiety is an antibody to CD30. In embodiments, the therapeutic moiety is an antibody to CD33. In embodiments, the therapeutic moiety is an antibody to CD38. In embodiments, the therapeutic moiety is an antibody to CD70. In embodiments, the therapeutic moiety is an antibody to PSCA. In embodiments, the therapeutic moiety is an antibody to PSMA. In embodiments, the therapeutic moiety is folate, cholesterol, or N-acetylgalactosamine (GalNac). In embodiments, the antibody or fragment thereof is capable of being cleaved or otherwise released from the 6tdG-oligonucleotide in vivo.

In embodiments, the therapeutic moiety is a STAT inhibitor moiety. In embodiments, the STAT inhibitor moiety is linked to the 3′ end of the 6tdG-oligonucleotide. In embodiments, the 5′ end of the STAT inhibitor moiety is covalently bonded to the 3′ end of the 6tdG-oligonucleotide. In embodiments, the 3′ end of the STAT inhibitor moiety is covalently bonded to the 3′ end of the 6tdG-oligonucleotide. In embodiments, the STAT inhibitor moiety is a STAT1 inhibitor. In embodiments, the STAT inhibitor moiety is a STAT2 inhibitor. In embodiments, the STAT inhibitor moiety is a STAT3 inhibitor. In embodiments, the STAT inhibitor moiety is a STAT4 inhibitor. In embodiments, the STAT inhibitor moiety is a STAT5A inhibitor. In embodiments, the STAT inhibitor moiety is a STAT5B inhibitor. In embodiments, the STAT inhibitor moiety is a STAT6 inhibitor. In embodiments, the STAT inhibitor moiety is a STAT7 inhibitor. In embodiments, the STAT inhibitor moiety is a STAT8 inhibitor. In embodiments, the STAT inhibitor moiety is a STAT7/8 inhibitor. In embodiments, the STAT inhibitor moiety is a STAT9 inhibitor.

In embodiments, the 6tdG-CpG oligodeoxynucleotide further comprises a exonuclease resistant moiety. In embodiments, the 6tdG-CpG oligodeoxynucleotide further comprises a exonuclease resistant moiety linked to the 3′ end of the 6tdG-CpG oligodeoxynucleotide. The exonuclease resistant moiety can be any known in the art. In embodiments, the exonuclease resistant moiety comprises a hydroxyalkyl-terminated monophosphate group, a hydroxyalkyl-terminated diphosphate group, a hydroxyalkyl-terminated triphosphate group, 2′-O-aminopropyl group, a 2′-constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, a locked nucleic acid (LNA) modification (e.g., the ribose is modified with an extra bridge connecting the 2′ oxygen and 4′ carbon), a dideoxy modification (e.g., the 2′ and 3′ positions on the ribose lack hydroxyl groups), an inverted deoxybasic modification, or a combination of two or more thereof.

In embodiments, the exonuclease resistant moiety comprises a hydroxyalkyl-terminated monophosphate group, a hydroxyalkyl-terminated diphosphate group, a hydroxyalkyl-terminated triphosphate group. In embodiments, the exonuclease resistant moiety comprises a hydroxyalkyl-terminated monophosphate group. In embodiments, the exonuclease resistant moiety comprises a hydroxyalkyl-terminated diphosphate group. In embodiments, the exonuclease resistant moiety comprises a hydroxyalkyl-terminated triphosphate group. In embodiments, the exonuclease resistant moiety is —OP(═O)(OH)—O—(CH₂)ₓOH, and x is an integer from 1 to 20. In embodiments, the exonuclease resistant moiety is —[OP(═O)(OH)]₂—O(CH₂)ₓOH, and x is an integer from 1 to 20. In embodiments, the exonuclease resistant moiety is —[OP(═O)(OH)]₃—O(CH₂)ₓOH, and x is an integer from 1 to 20. In embodiments, x is an integer from 1 to 15. In embodiments, x is an integer from 1 to 10. In embodiments, x is an integer from 1 to 6. In embodiments, x is 1. In embodiments, x is 2. In embodiments, x is 3. In embodiments, x is 4. In embodiments, x is 5. In embodiments, x is 6.

In embodiments, the exonuclease resistant moiety is a modified nucleotide or a nucleic acid comprising a modified nucleotide. In embodiments, the exonuclease resistant moiety is a modified nucleotide. In embodiments, the exonuclease resistant moiety is a nucleic acid comprising a modified nucleotide. In embodiments, the nucleic acid comprises one or more modified nucleotides. In embodiments, the nucleic acid comprises a modified nucleotide time at the 3′ end. In embodiments, the modified nucleotide comprises a hydroxyalkyl-terminated monophosphate group, a hydroxyalkyl-terminated diphosphate group, a hydroxyalkyl-terminated triphosphate group, a dideoxy modification, an inverted deoxybasic modification, 2′-O-aminopropyl group, a 2′ constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, or a locked nucleic acid modification. In embodiments, the modified nucleotide comprises a hydroxyalkyl-terminated monophosphate group. In embodiments, the modified nucleotide comprises a hydroxyalkyl-terminated diphosphate group. In embodiments, the modified nucleotide comprises a hydroxyalkyl-terminated triphosphate group. In embodiments, the modified nucleotide comprises a 2′-O-aminopropyl group. In embodiments, the modified nucleotide comprises a 2′-constrained ethyl group. In embodiments, the modified nucleotide comprises a 2′-fluoro group. In embodiments, the modified nucleotide comprises a 2′-O-methyl group. In embodiments, the modified nucleotide comprises a 2′-deoxy-2′fluoro group. In embodiments, the modified nucleotide comprises a 2′-O-methoxyethyl group. In embodiments, the modified nucleotide comprises a 2′-O-allyl group. In embodiments, the modified nucleotide comprises a 2′-O-propyl group. In embodiments, the modified nucleotide comprises a 2′-O-pentyl group. In embodiments, the modified nucleotide comprises a locked nucleic acid (LNA) modification. In embodiments, the modified nucleotide comprises a dideoxy modification. In embodiments, the modified nucleotide comprises an inverted deoxybasic modification.

The disclosure provides 6tdG-CpG oligodeoxynucleotides comprising at least one 6-thio-2′-deoxyguanosine residue. In embodiments, the 6-thio-2′-deoxyguanosine residue replaces at least one guanosine residue in the CpG oligodeoxynucleotide. In embodiments, the 6-thio-2′-deoxyguanosine residue is an additional nucleic acid residue in a CpG oligodeoxynucleotide. In embodiments, the 6tdG-CpG oligodeoxynucleotide comprises one 6-thio-2′-deoxyguanosine residues. In embodiments, the 6tdG-CpG oligodeoxynucleotide comprises two 6-thio-2′-deoxyguanosine residues. In embodiments, the 6tdG-CpG oligodeoxynucleotide comprises three 6-thio-2′-deoxyguanosine residues. In embodiments, the 6tdG-CpG oligodeoxynucleotide comprises four 6-thio-2′-deoxyguanosine residues. In embodiments, the 6tdG-CpG oligodeoxynucleotide comprises five 6-thio-2′-deoxyguanosine residues. In embodiments, the 6tdG-CpG oligodeoxynucleotide comprises six 6-thio-2′-deoxyguanosine residues. In embodiments, the 6tdG-CpG oligodeoxynucleotide comprises at least five 6-thio-2′-deoxyguanosine residues. In embodiments, the 6tdG-CpG oligodeoxynucleotide comprises at least six 6-thio-2′-deoxyguanosine residues. In embodiments, the at least one 6-thio-2′-deoxyguanosine residue is within 10 nucleic acids of the 3′ end of the 6tdG-CpG oligodeoxynucleotide. In embodiments, the at least one 6-thio-2′-deoxyguanosine residue is within 8 nucleic acids of the 3′ end of the 6tdG-CpG oligodeoxynucleotide. In embodiments, the at least one 6-thio-2′-deoxyguanosine residue is within 6 nucleic acids of the 3′ end of the 6tdG-CpG oligodeoxynucleotide. In embodiments, the 6tdG-CpG oligodeoxynucleotide comprises from about 10 nucleic acid residues to about 40 nucleic acid residues. In embodiments, the 6tdG-CpG oligodeoxynucleotide comprises from about 10 nucleic acid residues to about 35 nucleic acid residues. In embodiments, the 6tdG-CpG oligodeoxynucleotide comprises from about 12 nucleic acid residues to about 32 nucleic acid residues. In embodiments, the 6tdG-CpG oligodeoxynucleotide comprises from about 15 nucleic acid residues to about 30 nucleic acid residues. In embodiments, the 6tdG-CpG oligodeoxynucleotide comprises from about 15 nucleic acid residues to about 28 nucleic acid residues. In embodiments, the 6tdG-CpG oligodeoxynucleotide comprises from about 15 nucleic acid residues to about 25 nucleic acid residues. In embodiments, the 6tdG-CpG oligodeoxynucleotide comprises from about 15 nucleic acid residues to about 24 nucleic acid residues. In embodiments, the 6tdG-CpG oligodeoxynucleotide comprises from about 15 nucleic acid residues to about 23 nucleic acid residues. In embodiments, the 6tdG-CpG oligodeoxynucleotide comprises from about 15 nucleic acid residues to about 22 nucleic acid residues. In embodiments, the 6tdG-CpG oligodeoxynucleotide comprises from about 15 nucleic acid residues to about 20 nucleic acid residues.

In embodiments, the 6tdG-CpG oligodeoxynucleotide described herein or the CpG oligodeoxynucleotide described herein is a CpG-A ODN, a CpG-B ODN, a CpG-C ODN, or a combination of two or more thereof. In embodiments, the 6tdG-CpG oligodeoxynucleotide or the CpG oligodeoxynucleotide is a CpG-A ODN. In embodiments, the 6tdG-CpG oligodeoxynucleotide or the CpG oligodeoxynucleotide is a CpG-B ODN. In embodiments, the 6tdG-CpG oligodeoxynucleotide or the CpG oligodeoxynucleotide is a CpG-C ODN. In embodiments, the 6tdG-CpG oligodeoxynucleotide or the CpG oligodeoxynucleotide is CpG ODN D19, CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof. In embodiments, the 6tdG-CpG oligodeoxynucleotide or the CpG oligodeoxynucleotide is CpG ODN D19. In embodiments, the 6tdG-CpG oligodeoxynucleotide or the CpG oligodeoxynucleotide is CpG ODN 1585. In embodiments, the 6tdG-CpG oligodeoxynucleotide or the CpG oligodeoxynucleotide is CpG ODN 2216. In embodiments, the 6tdG-CpG oligodeoxynucleotide or the CpG oligodeoxynucleotide is CpG ODN 2336. In embodiments, the 6tdG-CpG oligodeoxynucleotide or the CpG oligodeoxynucleotide is CpG ODN 1668. In embodiments, the CpG ODN is CpG ODN 1826. In embodiments, the 6tdG-CpG oligodeoxynucleotide or the CpG oligodeoxynucleotide is CpG ODN 2006. In embodiments, the CpG ODN is CpG ODN 2007. In embodiments, the 6tdG-CpG oligodeoxynucleotide or the CpG oligodeoxynucleotide is CpG ODN BW006. In embodiments, the CpG ODN is CpG ODN D-SL01. In embodiments, the CpG ODN is CpG ODN 2395. In embodiments, the 6tdG-CpG oligodeoxynucleotide or the CpG oligodeoxynucleotide is CpG ODN CpG ODN M362. In embodiments, the 6tdG-CpG oligodeoxynucleotide or the CpG oligodeoxynucleotide is CpG ODN D-SL03. In embodiments, the 6tdG-CpG oligodeoxynucleotide or the CpG oligodeoxynucleotide comprises one or more phosphorothioate linkages.

In embodiments, the 6tdG-CpG oligodeoxynucleotide or the CpG oligodeoxynucleotide comprises a CpG ODN nucleic acid sequence listed in Table 1. In embodiments, the 6tdG-CpG oligodeoxynucleotide or the CpG oligodeoxynucleotide listed in Table 1 have one or more phosphorothioate linkages.

TABLE 1

| | SEQ ID | |
|---|---|---|
| NAME | NO: | SEQUENCE 5′-3′ |
| ODN D19 | 15 | GGTGCATCGATGCAGGGGGG |
| GpC(A)-ODN | 16 | GGTGCATGCATGCAGGGGGG |
| CpG(B)-ODN | 17 | TCCATGACGTTCCTGATGCT |
| ODN 1585 | 18 | GGGGTCAACGTTGAGGGGGG |
| ODN 2216 | 19 | GGGGGACGATCGTCGGGGGG |
| ODN 2336 | 20 | GGGGACGACGTCGTGGGGGGG |
| ODN 1668 | 21 | TCCATGACGTTCCTGATGCT |
| ODN 1826 | 22 | TCCATGACGTTCCTGACGTT |
| ODN 2006 | 23 | TCGTCGTTTTGTCGTTTTGTCGTT |
| ODN 2007 | 24 | TCGTCGTTGTCGTTTTGTCGTT |
| ODN 2395 | 25 | TCGTCGTTTTCGGCGCGCGCCG |
| ODN M362 | 26 | TCGTCGTCGTTCGAACGACGTTGAT |

In embodiments, the 6tdG-CpG oligodeoxynucleotide or the CpG oligodeoxynucleotide comprises a CpG-ODN nucleic acid sequence listed in Table 2, wherein the asterisk (*) indicates a phosphorothioate linkage.

TABLE 2

| | SEQ ID | SEQUENCE 5′-3′ |
|---|---|---|
| NAME | NO: | (*= phosphorothioate linkage) |
| ODN D19 | 27 | G*G*TGCATCGATGCAGG*G*G*G*G |
| ODN D19 | 28 | G*G*TGCATCGATGCAGG*G*G*G*G* |
| GpC(A)- | 29 | G*G*TGCATGCATGCAGG*G*G*G*G |
| ODN | | |
| D19-PS | 30 | G*G*T*G*C*A*T*C*G*A*T*G*C* |
| | | A*G*G*G*G*G*G |
| CpG(B)- | 31 | T*C*C*A*T*G*A*C*G*T*T*C*C* |
| ODN | | T*G*A*T*G*C*T |
| ODN 1585 | 32 | G*G*GGTCAACGTTGAG*G*G*G*G*G |
| ODN 1585 | 33 | G*GGGTCAACGTTGAG*G*G*G*G*G |
| ODN 2216 | 34 | G*G*GGGACGATCGTCG*G*G*G*G*G |
| ODN 2216 | 35 | G*GGGGACGATCGTCG*G*G*G*G*G |
| ODN 2336 | 36 | G*G*G*GACGACGTCGTGG*G*G*G*G*G |
| ODN 2336 | 37 | G*G*GGACGACGTCGTGG*G*G*G*G*G |
| ODN 1668 | 38 | T*C*C*A*T*G*A*C*G*T*T*C*C* |
| | | T*G*A*T*G*C*T |
| ODN 1826 | 39 | T*C*C*A*T*G*A*C*G*T*T*C*C* |
| | | T*G*A*C*G*T*T |
| ODN 2006 | 40 | T*C*G*T*C*G*T*T*T*T*G*T*C* |
| | | G*T*T*T*T*G*T*C*G*T*T |
| ODN 2007 | 41 | T*C*G*T*C*G*T*T*G*T*C*G*T* |
| | | T*T*T*G*T*C*G*T*T |
| ODN 2395 | 42 | T*C*G*T*C*G*T*T*T*T*C*G*G* |
| | | C*G*C*G*C*G*C*C*G |
| ODN M362 | 43 | T*C*G*T*C*G*T*C*G*T*T*C*G* |
| | | A*A*C*G*A*C*G*T*T*G*A*T |

In embodiments, the 6tdG-CpG oligodeoxynucleotide further comprises (i) from 1 to 12 adenine residues; (ii) from 1 to 12 thymine residues; (iii) a nucleic acid comprising from 1 to about 12 adenine residues and from 1 to about 12 thymine residue; or (iv) a combination of two or more of the foregoing. In embodiments, (i), (ii), or (iii) are interspersed between one or more 6-thio-2′-deoxyguanosine residues. In embodiments, the CpG oligodeoxynucleotide comprises a 6-thio-2′-deoxyguanosine mixmer.

The disclosure provides compounds of Formula (I):

(I)
5′-R³-R¹-L¹-L²-L³-L⁴-L⁵-L⁶-R²,

where the substituents are as defined herein.

The disclosure provides compounds of Formula (II):

(II)
5′-R¹-L¹-L²-L³-L⁴-L⁵-L⁶-R²,

where the substituents are as defined herein.

The disclosure provides compounds of Formula (III):

(III)
5′-R³-R¹-L¹-L²-L³-L⁴-L⁵-L⁶-(L⁷)ₘL⁸-R²,

where the substituents are as defined herein.

R¹ is SEQ ID NO:1, SEQ ID NO:2, SEQ ID NO:3, SEQ ID NO:4, SEQ ID NO:5, SEQ ID NO:6, SEQ ID NO:7, SEQ ID NO: 8, or SEQ ID NO:9. In embodiments, R¹ is SEQ ID NO: 1. In embodiments, R¹ is SEQ ID NO:2. In embodiments, R¹ is SEQ ID NO:3. In embodiments, R¹ is SEQ ID NO:4. In embodiments, R¹ is SEQ ID NO:5. In embodiments, R¹ is SEQ ID NO:6. In embodiments, R¹ is SEQ ID NO: 7. In embodiments, R¹ is SEQ ID NO:8. In embodiments, R¹ is SEQ ID NO:9.

SEQ ID NO: 1 refers to 5′-GGTGCATCGATGCA-.
SEQ ID NO:2 refers to 5′-GGTGCATGCATGCA-.
SEQ ID NO:3 refers to 5′-GGGGTCAACGTTGA-.
SEQ ID NO:4 refers to 5′-GGGGGACGATCGTC-.
SEQ ID NO:5 refers to 5′-GGGGACGACGTCGTG-.
SEQ ID NO:6 refers to 5′-TCGTCGTTTT.
SEQ ID NO:7 refers to 5′-TCGTCGTTTTGTCGTTTTGTCGTT.
SEQ ID NO:8 refers to 5′-TCGTCGTTTTCGGCGGCCGCCG.
SEQ ID NO:9 refers to 5′-TCGTCGTTTTCGGCGCGCGCCG.

In embodiments, one or more internucleotide linkages in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9 is a phosphorothioated internucleotide linkage. In embodiments, SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9 comprise at least one phosphorothioated internucleotide linkage. In embodiments, SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9 comprise at least two phosphorothioated internucleotide linkages. In embodiments, SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9 comprise at least three phosphorothioated internucleotide linkage. In embodiments, R¹ is SEQ NO:10, SEQ ID NO:11, SEQ ID NO:12, SEQ ID NO:13, or SEQ ID NO:14. In embodiments, R¹ is SEQ NO:10. In embodiments, R¹ is SEQ NO:11. In embodiments, R¹ is SEQ NO:12. In embodiments, R¹ is SEQ NO:13. In embodiments, R¹ is SEQ NO:14. In embodiments, the 3′ end of R¹ is bonded to L¹. In embodiments, the 5′ end of R¹ is bonded to L¹.

SEQ ID NO: 10 refers to 5′-G*G*TGCATCGATGCA-.
SEQ ID NO: 11 refers to 5′-G*G*TGCATGCATGCA-.
SEQ ID NO: 12 refers to 5′-G*G*GGTCAACGTTGA-.
SEQ ID NO: 13 refers to 5′-G*G*GGGACGATCGTC-.
SEQ ID NO: 14 refers to 5′-G*G*G*GACGACGTCGTG-.

L¹, L², L³, L⁴, L⁵, and L⁶ are each independently a bond, a guanosine residue, a deoxyguanosine residue, or a 6-thio-2′-deoxyguanosine residue; wherein at least one of L¹, L², L³, L⁴, L⁵, and L⁶ is a 6-thio-2′-deoxyguanosine residue. In embodiments, L¹, L², L³, L⁴, L⁵, and L⁶ are each independently a bond, a guanosine residue, a deoxyguanosine residue, or a 6-thio-2′-deoxyguanosine residue; wherein at least two of L¹, L², L³, L⁴, In embodiments, L⁵, and L⁶ are a 6-thio-2′-deoxyguanosine residue. In embodiments, L¹, L², L³, L⁴, L⁵, and L⁶ are each independently a bond, a guanosine residue, a deoxyguanosine residue, or a 6-thio-2′-deoxyguanosine residue; wherein at least three of L¹, L², L³, L⁴, L⁵, and L⁶ are a 6-thio-2′-deoxyguanosine residue. In embodiments, L¹, L², L³, L⁴, L⁵, and L⁶ are each independently a bond, a guanosine residue, a deoxyguanosine residue, or a 6-thio-2′-deoxyguanosine residue; wherein at least four of L¹, L², L³, L⁴, L⁵, and L⁶ are a 6-thio-2′-deoxyguanosine residue. In embodiments, L¹, L², L³, L⁴, L⁵, and L⁶ are each independently a bond, a guanosine residue, a deoxyguanosine residue, or a 6-thio-2′-deoxyguanosine residue; wherein at least five of L¹, L², L³, L⁴, L⁵, and L⁶ are a 6-thio-2′-deoxyguanosine residue. L¹, L², L³, L⁴, L⁵, and L⁶ are each independently a guanosine residue or a 6-thio-2′-deoxyguanosine residue; wherein at least one of L¹, L², L³, L⁴, L⁵, and L⁶ is a 6-thio-2′-deoxyguanosine residue. In embodiments, L¹, L², L³, L⁴, L⁵, and L⁶ are each independently a guanosine residue or a 6-thio-2′-deoxyguanosine residue; wherein at least two of L¹, L², L³, L⁴, In embodiments, L⁵, and L⁶ are a 6-thio-2′-deoxyguanosine residue. In embodiments, L¹, L², L³, L⁴, L⁵, and L⁶ are each independently a guanosine residue or a 6-thio-2′-deoxyguanosine residue; wherein at least three of L¹, L², L³, L⁴, L⁵, and L⁶ are a 6-thio-2′-deoxyguanosine residue. In embodiments, L¹, L², L³, L⁴, L⁵, and L⁶ are each independently a guanosine residue or a 6-thio-2′-deoxyguanosine residue; wherein at least four of L¹, L², L³, L⁴, L⁵, and L⁶ are a 6-thio-2′-deoxyguanosine residue. In embodiments, L¹, L², L³, L⁴, L⁵, and L⁶ are each independently a guanosine residue or a 6-thio-2′-deoxyguanosine residue; wherein at least five of L¹, L², L³, L⁴, L⁵, and L⁶ are a 6-thio-2′-deoxyguanosine residue. In embodiments, L⁶ is a guanosine residue and L¹, L², L³, L⁴, and L⁵ are a 6-thio-2′-deoxyguanosine residue. In embodiments, L¹, L², L³, L⁴, L⁵, and L⁶ are a 6-thio-2′-deoxyguanosine residue.

L⁷ is independently a guanosine residue or a 6tdG residue. In embodiments, L⁷ is independently a 6tdG residue. m is an integer from 0 to 12. In embodiments, m is 0. In embodiments, m is 1. In embodiments, m is 2. In embodiments, m is 3. In embodiments, m is 4. In embodiments, m is 5. In embodiments, m is 6.

In embodiments, R² is hydrogen, a phosphate moiety, an exonuclease resistant moiety, a therapeutic moiety, or a detectable moiety. In embodiments, R² is hydrogen. In embodiments, R² is a phosphate moiety. In embodiments, the phosphate moiety is a monophosphate moiety, a diphosphate moiety, or a triphosphate moiety. In embodiments, the phosphate moiety is a monophosphate moiety comprising a phosphorothioate internucleotide linkage, a diphosphate moiety comprising a phosphorothioate internucleotide linkage, or a triphosphate moiety comprising a phosphorothioate internucleotide linkage.

In embodiments, R³ is absent, a therapeutic moiety, or a detectable moiety. In embodiments, R³ is absent. In embodiments, R³ is a therapeutic moiety. In embodiments, R³ is a detectable moiety. In embodiments, R² is hydrogen, a phosphate moiety, an exonuclease resistant moiety, a therapeutic moiety, or a detectable moiety, and R³ is absent, a therapeutic moiety, or a detectable moiety. In embodiments, R² is hydrogen, a phosphate moiety, an exonuclease resistant moiety, a therapeutic moiety, or a detectable moiety, and R³ is absent. R² is hydrogen, a phosphate moiety, an exonuclease resistant moiety, a therapeutic moiety, or a detectable moiety, and R³ is a therapeutic moiety. R² is hydrogen, a phosphate moiety, an exonuclease resistant moiety, a therapeutic moiety, or a detectable moiety, and R³ is a detectable moiety.

In embodiments, R² is an exonuclease resistant moiety. In embodiments, the exonuclease resistant moiety is hydroxyalkyl-terminated phosphate group. In embodiments, the exonuclease resistant moiety is —OP(═O)(OH)—O—(CH₂)ₓOH, and x is an integer from 1 to 20. In embodiments, x is an integer from 1 to 10. In embodiments, x is an integer from 1 to 6. In embodiments, x is 1. In embodiments, x is 2. In embodiments, x is 3. In embodiments, x is 4. In embodiments, x is 5. In embodiments, x is 6. In embodiments, the exonuclease resistant moiety comprises a modified nucleotide (e.g., a single nucleotide) or a nucleic acid comprising a modified nucleotide (e.g., a nucleic acid containing 2 to 12 nucleotides having at least one modified nucleotide or a nucleic acid containing 2 to 12 nucleotides where at least the 3′end nucleotide is a modified nucleotide). The modified nucleotide can contain a modified base, a modified sugar, a modified phosphate group, or a combination of two or more thereof. In embodiments the modified nucleotide comprises a phosphorothioate group, a dideoxy modification, an inverted deoxybasic modification, 2′-O-aminopropyl group, a 2′ constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, or a locked nucleic acid modification.

In embodiments, at least one of R² and R³ is a therapeutic moiety. In embodiments, R² is a therapeutic moiety and R³ is absent. In embodiments, R³ is a therapeutic moiety and R² is absent. In embodiments, R² and R³ are a therapeutic moiety. In embodiments, R² and R³ are the same therapeutic moiety. In embodiments, R² and R³ are each a different therapeutic moiety. In embodiments, the therapeutic moiety is a compound, such as small molecule, antibody, DNA aptamer, or RNA aptamer, that binds to a target molecule. In such an embodiment, the therapeutic moiety can be referred to as a “targeting moiety” because it binds to a target molecule. The target molecule can be a receptor (e.g., a cell surface receptor) or a protein or enzyme (e.g., a protein or enzyme that binds to a cell surface receptor). Thus, the therapeutic moiety (optionally referred to as a targeting moiety) can bind directly to a cell surface receptor or bind to a protein or enzyme in order to prevent that protein or enzyme from binding to a cell surface receptor or to interfere with a signaling pathway. In embodiments, the therapeutic moiety binds to a cell surface receptor to inhibit the activity of the cell surface receptor. In embodiments, the therapeutic moiety binds to a protein or enzyme to inhibit the ability of that protein or enzyme to bind to a cell surface receptor. In embodiments, the therapeutic moiety binds to a protein or enzyme to inhibit or otherwise interfere with a signaling pathway. The therapeutic moiety can be released from the 6tdG-oligonucleotide prior to binding to a target molecule or can remain bonded to the 6tdG-oligonucleotide when binding to the target molecule. Exemplary therapeutic moieties include monoclonal antibodies, folate, cholesterol, N-acetylgalactosamine, and STAT inhibitors. In embodiments, the monoclonal antibody is capable of binding to HER2, EFGR, VEGFR, HGFR, SLAMF7, GD2, CD19, CD20, CD30, CD33, CD38, CD70, PSCA, or PSMA. In embodiments, the therapeutic moiety is a nucleic acid. In embodiments, the therapeutic moiety is a small molecule. In embodiments, the therapeutic moiety is an antibody. In embodiments, the therapeutic moiety is a DNA aptamer. In embodiments, the therapeutic moiety is an RNA aptamer. In embodiments, the therapeutic moiety is a STAT inhibitor. In embodiments, the therapeutic moiety is a STAT3 inhibitor. In embodiments, the therapeutic moiety is an antibody to HER2. In embodiments, the therapeutic moiety is an antibody to EGFR. In embodiments, the therapeutic moiety is an antibody to VEGFR. In embodiments, the therapeutic moiety is an antibody to HGFR. In embodiments, the therapeutic moiety is an antibody to SLAMF7. In embodiments, the therapeutic moiety is an antibody to GD2. In embodiments, the therapeutic moiety is an antibody to CD19. In embodiments, the therapeutic moiety is an antibody to CD20. In embodiments, the therapeutic moiety is an antibody to CD30. In embodiments, the therapeutic moiety is an antibody to CD33. In embodiments, the therapeutic moiety is an antibody to CD38. In embodiments, the therapeutic moiety is an antibody to CD70. In embodiments, the therapeutic moiety is an antibody to PSCA. In embodiments, the therapeutic moiety is an antibody to PSMA. In embodiments, the therapeutic moiety is folate, cholesterol, or N-acetylgalactosamine (GalNac). In embodiments, the therapeutic moiety is capable of being cleaved or otherwise released from the 6tdG-oligonucleotide in vivo.

In embodiments, the 6tdG-oligonucleotides described herein further comprise a detectable moiety. In embodiments, at least one of R² and R³ is a detectable moiety. In embodiments, R² is a detectable moiety and R³ is absent. In embodiments, R³ is a detectable moiety and R² is absent. In embodiments, R² and R³ are a detectable moiety. In embodiments, R² and R³ are the same detectable moiety. In embodiments, R² and R³ are each a different detectable moiety.

A “detectable moiety” is a compound or composition detectable by appropriate means such as spectroscopic, photochemical, biochemical, immunochemical, chemical, magnetic resonance imaging, or other physical means. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, the detectable agent is a detectable fluorescent agent. In embodiments, the detectable agent is a detectable phosphorescent agent. In embodiments, the detectable agent is a detectable radioactive agent. In embodiments, the detectable agent is a detectable metalloenzyme. In embodiments, the detectable agent is a detectable colorimetric agent. In embodiments, the detectable agent is a detectable luminescent agent. In embodiments, the detectable agent is a detectable spectrophotometric agent. In embodiments, the detectable agent is a detectable metal-organic framework. In embodiments, the detectable agent comprises a fluorophore linked to biotin, avidin, or streptavidin. In embodiments, the detectable agent is a chemiluminescent agent. In embodiments, the detectable agent is a radionuclide. In embodiments, the detectable agent is a radioisotope. In embodiments, the detectable agent is a paramagnetic molecule or a paramagnetic nanoparticle.

### Pharmaceutical Compositions

In embodiments, the disclosure provides pharmaceutical compositions comprising: (i) an effective amount of the 6tdG-oligonucleotides described herein, including all embodiments thereof, and (ii) a pharmaceutically acceptable excipient. In embodiments, the disclosure provides pharmaceutical compositions comprising: (i) an effective amount of the 6tdG-oligonucleotides described herein, including all embodiments thereof, (ii) an effective amount of a therapeutic agent (e.g., a STAT inhibitor), and (iii) a pharmaceutically acceptable excipient. In embodiments, the disclosure provides pharmaceutical compositions comprising: (i) an effective amount of a CpG oligodeoxynucleotide, (ii) an effective amount of 6-thio-2′-deoxyguanosine, and (iii) a pharmaceutically acceptable excipient. In embodiments, the disclosure provides pharmaceutical compositions comprising: (i) an effective amount of a CpG oligodeoxynucleotide, (ii) an effective amount of 6-thio-2′-deoxyguanosine, (iii) an effective amount of a therapeutic agent (e.g., a STAT inhibitor), and (iv) a pharmaceutically acceptable excipient.

A “effective amount” is an amount sufficient for a compound of the disclosure to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound (e.g., oligonucleotide) that are capable of achieving the methods described herein, as measured using the methods described herein or known in the art.

As is known in the art, therapeutically effective amounts for use in humans can also be determined from animal models. For example, a dose for humans can be formulated to achieve a concentration that has been found to be effective in animals. The dosage in humans can be adjusted by monitoring the effectiveness of the compositions, neural stem cells, and vesicles described herein, and adjusting the dosage upwards or downwards. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent (e.g., oligonucleotide) sufficient to ameliorate the disorder, as described above. For example, for the given parameter, a therapeutically effective amount will show an increase or decrease of at least 10%, 25%, 50%, 75%, 90%, or at least 100%. Therapeutic efficacy can also be expressed as “-fold” increase or decrease. For example, a therapeutically effective amount can have at least a 2-fold, 5-fold, or more effect over a control.

Dosages may be varied depending upon the requirements of the patient and the compound used. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intra-tumoral, intravenous, intramuscular, intra-arteriole, intradermal, subcutaneous, intraperitoneal, intraventricular, and intracranial. In embodiments, the neural stem cells, vesicles or pharmaceutical compositions described herein are parenterally administered to a patient. In embodiments, the neural stem cells, vesicles or pharmaceutical compositions described herein are administered intra-tumorally to a patient. Other modes of delivery include, but are not limited to, the use of liposomal formulations, intravenous infusion, transdermal patches, etc. In embodiments, the administering does not include administration of any active agent other than the recited active agent.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

### Dose and Dosing Regimens

The dosage and frequency (single or multiple doses) of the active agents described herein, including all embodiments thereof, administered to a subject can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer and severity of such symptoms), kind of concurrent treatment, complications from the disease being treated or other health-related problems. Other therapeutic regimens or agents can be used in conjunction with the methods described herein. Adjustment and manipulation of established dosages (e.g., frequency and duration) are well within the ability of those skilled in the art.

For any active agents described herein, the effective amount can be initially determined from cell culture assays. Target concentrations will be those concentrations of active agents that are capable of achieving the methods described herein, as measured using the methods described herein or known in the art. As is known in the art, effective amounts of active agents for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the active agents may be varied depending upon the requirements of the patient. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Generally, treatment is initiated with smaller dosages which are less than the optimum dose of the active agents. Thereafter, the dosage is increased by small increments until the optimum effect under circumstances is reached. Dosage amounts and intervals can be adjusted individually to provide levels of the active agents effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active agents by considering factors such as compound potency, relative bioavailability, patient body weight, presence and severity of adverse side effects.

In embodiments, the oligonucleotide is administered to a patient at an amount of about 0.01 mg/kg to about 500 mg/kg. In aspects, the oligonucleotide is administered to a patient in an amount of about 0.01 mg/kg to about 300 mg/kg. It is understood that where the amount is referred to as “mg/kg,” the amount is milligram per kilogram body weight of the subject being administered with the oligonucleotide. In aspects, the oligonucleotide is administered in an amount from about 0.1 mg to about 1,000 mg per day, as a single or divided dose.

### Methods of Treatment

In embodiments, the disclosure provides methods of treating cancer in a patient in need thereof by administering to the patient in an effective amount of the 6tdG-oligonucleotides described herein, including all embodiments thereof. In embodiments, the disclosure provides methods of treating cancer in a patient in need thereof by administering to the patient in an effective amount of the 6tdG-CpG oligodeoxynucleotides described herein, including all embodiments thereof. In embodiments, the disclosure provides methods of treating cancer in a patient in need thereof by administering to the patient in an effective amount of the 6tdG-CpG oligodeoxynucleotides of Formula (I) described herein, including all embodiments thereof. In embodiments, the disclosure provides methods of treating cancer in a patient in need thereof by administering to the patient in an effective amount of the 6tdG-CpG oligodeoxynucleotides of Formula (II) described herein, including all embodiments thereof. In embodiments, the disclosure provides methods of treating cancer in a patient in need thereof by administering to the patient in an effective amount of the 6tdG-CpG oligodeoxynucleotides of Formula (III) described herein, including all embodiments thereof. In embodiments, the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control.

In embodiments, the disclosure provides methods of treating cancer in a patient in need thereof by administering to the patient in an effective amount of a pharmaceutical composition comprising a pharmaceutically acceptable excipient and the 6tdG-oligonucleotides described herein, including all embodiments thereof. In embodiments, the disclosure provides methods of treating cancer in a patient in need thereof by administering to the patient in an effective amount of a pharmaceutical composition comprising a pharmaceutically acceptable excipient and the 6tdG-CpG oligodeoxynucleotide described herein, including all embodiments thereof. In embodiments, the disclosure provides methods of treating cancer in a patient in need thereof by administering to the patient in an effective amount of a pharmaceutical composition comprising a pharmaceutically acceptable excipient and the 6tdG-CpG oligodeoxynucleotides of Formula (I) described herein, including all embodiments thereof. In embodiments, the disclosure provides methods of treating cancer in a patient in need thereof by administering to the patient in an effective amount of a pharmaceutical composition comprising a pharmaceutically acceptable excipient and the 6tdG-CpG oligodeoxynucleotides of Formula (II) described herein, including all embodiments thereof. In embodiments, the disclosure provides methods of treating cancer in a patient in need thereof by administering to the patient in an effective amount of a pharmaceutical composition comprising a pharmaceutically acceptable excipient and the 6tdG-CpG oligodeoxynucleotides of Formula (III) described herein, including all embodiments thereof. In embodiments, the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control.

In embodiments, the disclosure provides methods of treating cancer in a patient in need thereof by administering to the patient in an effective amount of (i) the 6tdG-oligodeoxynucleotides described herein, including all embodiments and embodiments thereof, and (ii) a STAT inhibitor. In embodiments, the disclosure provides methods of treating cancer in a patient in need thereof by administering to the patient in an effective amount of: (i) the 6tdG-CpG oligodeoxynucleotides described herein, including all embodiments and embodiments thereof, and (ii) a STAT inhibitor. In embodiments, the disclosure provides methods of treating cancer in a patient in need thereof by administering to the patient in an effective amount of: (i) the 6tdG-CpG oligodeoxynucleotides of Formula (I) described herein, including all embodiments and embodiments thereof, and (ii) a STAT inhibitor. In embodiments, the disclosure provides methods of treating cancer in a patient in need thereof by administering to the patient in an effective amount of: (i) the 6tdG-CpG oligodeoxynucleotides of Formula (II) described herein, including all embodiments and embodiments thereof, and (ii) a STAT inhibitor. In embodiments, the disclosure provides methods of treating cancer in a patient in need thereof by administering to the patient in an effective amount of: (i) the 6tdG-CpG oligodeoxynucleotides of Formula (III) described herein, including all embodiments and embodiments thereof, and (ii) a STAT inhibitor. In embodiments, the disclosure provides methods of treating cancer in a patient in need thereof by administering to the patient in an effective amount of: (i) a pharmaceutical composition comprising a pharmaceutically acceptable excipient and the 6tdG-CpG oligodeoxynucleotide described herein, and (ii) a pharmaceutical composition comprising a STAT inhibitor and a pharmaceutically acceptable excipient. In embodiments, the disclosure provides methods of treating cancer in a patient in need thereof by administering to the patient in an effective amount of: (i) a pharmaceutical composition comprising a pharmaceutically acceptable excipient and the 6tdG-CpG oligodeoxynucleotides of Formula (I) described herein, and (ii) a pharmaceutical composition comprising a STAT inhibitor and a pharmaceutically acceptable excipient. In embodiments, the disclosure provides methods of treating cancer in a patient in need thereof by administering to the patient in an effective amount of: (i) a pharmaceutical composition comprising a pharmaceutically acceptable excipient and the 6tdG-CpG oligodeoxynucleotides of Formula (II) described herein, and (ii) a pharmaceutical composition comprising a STAT inhibitor and a pharmaceutically acceptable excipient. In embodiments, the disclosure provides methods of treating cancer in a patient in need thereof by administering to the patient in an effective amount of: (i) a pharmaceutical composition comprising a pharmaceutically acceptable excipient and the 6tdG-CpG oligodeoxynucleotides of Formula (III) described herein, and (ii) a pharmaceutical composition comprising a STAT inhibitor and a pharmaceutically acceptable excipient. In embodiments, the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control.

In embodiments, the disclosure provides methods of treating cancer in a patient in need thereof by administering to the patient in an effective amount of: (i) a CpG oligodeoxynucleotide, and (ii) 6-thio-2′-deoxyguanosine. In embodiments, the disclosure provides methods of treating cancer in a patient in need thereof by administering to the patient in an effective amount of: (i) a pharmaceutical composition comprising a pharmaceutically acceptable excipient and a CpG oligodeoxynucleotide, and (ii) a pharmaceutical composition comprising a 6-thio-2′-deoxyguanosine and a pharmaceutically acceptable excipient. In embodiments, the disclosure provides methods of treating cancer in a patient in need thereof by administering to the patient in an effective amount of a pharmaceutical composition comprising (a) a pharmaceutically acceptable excipient, (b) a CpG oligodeoxynucleotide, and (c) 6-thio-2′-deoxyguanosine. In embodiments, the CpG oligodeoxynucleotide a CpG-A ODN, a CpG-B ODN, a CpG-C ODN, or a combination of two or more thereof. In embodiments, the CpG ODN is a CpG-A ODN. In embodiments, the CpG ODN is a CpG-B ODN. In embodiments, the CpG ODN is a CpG-C ODN. In embodiments, the CpG ODN is CpG ODN D19, CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof. In embodiments, the CpG ODN is CpG ODN D19. In embodiments, the CpG ODN is CpG ODN 1585. In embodiments, the CpG ODN is CpG ODN 2216. In embodiments, the CpG ODN is CpG ODN 2336. In embodiments, the CpG ODN is CpG ODN 1668. In embodiments, the CpG ODN is CpG ODN 1826. In embodiments, the CpG ODN is CpG ODN 2006. In embodiments, the CpG ODN is CpG ODN 2007. In embodiments, the CpG ODN is CpG ODN BW006. In embodiments, the CpG ODN is CpG ODN D-SL01. In embodiments, the CpG ODN is CpG ODN 2395. In embodiments, the CpG ODN is CpG ODN CpG ODN M362. In embodiments, the CpG ODN is CpG ODN D-SL03. In embodiments, the CpG oligodeoxynucleotide is selected from the group consisting of SEQ ID NOS:15-43. In embodiments, the CpG oligodeoxynucleotide comprises one or more phosphorothioate linkages. In embodiments, the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control.

In embodiments, the disclosure provides methods of treating cancer in a patient in need thereof by administering to the patient in an effective amount of: (i) a CpG oligodeoxynucleotide, (ii) 6-thio-2′-deoxyguanosine, and (iii) a STAT inhibitor. In embodiments, the disclosure provides methods of treating cancer in a patient in need thereof by administering to the patient in an effective amount of: (i) a pharmaceutical composition comprising a pharmaceutically acceptable excipient and a CpG oligodeoxynucleotide, (ii) a pharmaceutical composition comprising a 6-thio-2′-deoxyguanosine and a pharmaceutically acceptable excipient, and (iii) a pharmaceutical composition comprising a STAT inhibitor and a pharmaceutically acceptable excipient. In embodiments, the disclosure provides methods of treating cancer in a patient in need thereof by administering to the patient in an effective amount of (i) a pharmaceutical composition comprising (a) a pharmaceutically acceptable excipient, (b) a CpG oligodeoxynucleotide, and (c) 6-thio-2′-deoxyguanosine; and (ii) a pharmaceutical composition comprising a STAT inhibitor and a pharmaceutically acceptable excipient. In embodiments, the disclosure provides methods of treating cancer in a patient in need thereof by administering to the patient in an effective amount of (i) a pharmaceutical composition comprising (a) a pharmaceutically acceptable excipient, (b) a CpG oligodeoxynucleotide, and (c) a STAT inhibitor; and (ii) a pharmaceutical composition comprising 6-thio-2′-deoxyguanosine and a pharmaceutically acceptable excipient. In embodiments, the disclosure provides methods of treating cancer in a patient in need thereof by administering to the patient in an effective amount of (i) a pharmaceutical composition comprising (a) a pharmaceutically acceptable excipient, (b) 6-thio-2′-deoxyguanosine, and (c) a STAT inhibitor; and (ii) a pharmaceutical composition comprising a CpG oligodeoxynucleotide and a pharmaceutically acceptable excipient. In embodiments, the disclosure provides methods of treating cancer in a patient in need thereof by administering to the patient in an effective amount of a pharmaceutical composition comprising (a) a pharmaceutically acceptable excipient, (b) 6-thio-2′-deoxyguanosine, (c) a STAT inhibitor, and (d) a CpG oligodeoxynucleotide. In embodiments, the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control.

In embodiments, the methods described herein are for treating prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma. In embodiments, the methods described herein are for treating prostate cancer. In embodiments, the methods described herein are for treating colon cancer. In embodiments, the methods described herein are for treating non-small cell lung cancer. In embodiments, the methods described herein are for treating liver cancer. In embodiments, the methods described herein are for treating bladder cancer. In embodiments, the methods described herein are for treating pancreatic cancer. In embodiments, the methods described herein are for treating breast cancer. In embodiments, the methods described herein are for treating ovarian cancer. In embodiments, the methods described herein are for treating brain cancer. In embodiments, the methods described herein are for treating glioma. In embodiments, the methods described herein are for treating melanoma. In embodiments, the methods described herein are for treating head and neck cancer. In embodiments, the methods described herein are for treating renal cancer. In embodiments, the methods described herein are for treating leukemia. In embodiments, the methods described herein are for treating acute myeloid leukemia (AML). In embodiments, the methods described herein are for treating lymphoma. In embodiments, the methods described herein are for treating B-cell lymphoma. In embodiments, the methods described herein are for treating diffuse large B-cell lymphoma (DLBCL).

In embodiments, the cancer expresses telomerase reverse transcriptase. In embodiments, the cancer expresses elevated levels of telomerase reverse transcriptase. In embodiments, the cancer expresses elevated levels of telomerase reverse transcriptase relative to a control. In embodiments, the cancer expressing (e.g., elevated levels of) telomerase reverse transcriptase is ovarian cancer, prostate cancer, colon cancer, liver cancer, bladder cancer, head and neck cancer, renal cancer, leukemia, or lymphoma. In embodiments, the cancer expressing (e.g., elevated levels of) telomerase reverse transcriptase is prostate cancer, colon cancer, liver cancer, bladder cancer, head and neck cancer, renal cancer, leukemia, or lymphoma. In embodiments, the cancer expressing (e.g., elevated levels of) telomerase reverse transcriptase is prostate cancer, colon cancer, liver cancer, bladder cancer, head and neck cancer, renal cancer, acute myeloid leukemia, or lymphoma. In embodiments, the cancer expressing (e.g., elevated levels of) telomerase reverse transcriptase is prostate cancer. In embodiments, the prostate cancer is castration-resistant prostate cancer. In embodiments, the prostate cancer is non-metastatic castration-resistant prostate cancer. In embodiments, the cancer expressing (e.g., elevated levels of) telomerase reverse transcriptase is colon cancer. In embodiments, the cancer expressing (e.g., elevated levels of) telomerase reverse transcriptase is liver cancer. In embodiments, the cancer expressing (e.g., elevated levels of) telomerase reverse transcriptase is bladder cancer. In embodiments, the cancer expressing (e.g., elevated levels of) telomerase reverse transcriptase is a head and neck cancer. In embodiments, the head and neck cancer is head and neck squamous cell carcinoma that is positive for human papilloma virus. In embodiments, the head and neck cancer is head and neck squamous cell carcinoma that is positive for human papilloma virus 16. In embodiments, the cancer expressing (e.g., elevated levels of) telomerase reverse transcriptase is renal cancer. In embodiments, the cancer expressing (e.g., elevated levels of) telomerase reverse transcriptase is leukemia. In embodiments, the leukemia is myeloid leukemia. In embodiments, the leukemia is acute myeloid leukemia. In embodiments, the cancer expressing (e.g., elevated levels of) telomerase reverse transcriptase is lymphoma. In embodiments, the cancer expressing (e.g., elevated levels of) telomerase reverse transcriptase is B-cell lymphoma. In embodiments, the cancer expressing (e.g., elevated levels of) telomerase reverse transcriptase is diffuse large B-cell lymphoma (DLBCL). In embodiments, the cancer expressing (e.g., elevated levels of) telomerase reverse transcriptase is ovarian cancer.

The term “cancer” refers to all types of cancer, neoplasm or malignant tumors found in mammals (e.g. humans), including leukemias, lymphomas, carcinomas and sarcomas. Exemplary cancers that may be treated with compounds, nucleic acids, and pharmaceutical compositions described herein include leukemia (e.g., acute myeloid leukemia (“AML”) or chronic myeloid leukemia (“CML”)) brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's disease, and Non-Hodgkin's lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, chronic myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large B-cell lymphoma (DLBCL), Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Treating” or “treatment” as used herein (and as well-understood in the art) includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. Treatment may inhibit the disease's spread; relieve the disease's symptoms, fully or partially remove the disease's underlying cause, shorten a disease's duration, or do a combination of these things. “Treating” does not include preventing.

“Patient” or “subject in need thereof” refers to a living organism suffering from or prone to a disease or condition that can be treated by administration of a pharmaceutical composition as provided herein. Non-limiting examples include humans, other mammals, bovines, horses, dogs, cats, monkeys, and other non-mammalian animals. In embodiments, a patient is human.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

“Coadminister” means that compounds, nucleic acids, or pharmaceutical composition described herein is administered at the same time, just prior to, or just after the administration of one or more additional anti-inflammatory agents, anti-cancer agents and/or radiation treatment. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

In embodiments of the methods of treating cancer described herein, the methods may further comprising administering to the patient an effective amount of an additional (different) anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. Exemplary anti-cancer agents include antibodies, small molecules, large molecules, and combinations thereof. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, erbulozole (i.e. R-55104), dolastatin 10 (i.e. DLS-10 and NSC-376128), mivobulin isethionate (i.e. as CI-980), vincristine, NSC-639829, Discodermolide (i.e. as NVP-XX-A-296), ABT-751 (Abbott, i.e. E-7010), altorhyrtins (e.g. Altorhyrtin A and Altorhyrtin C), spongistatins (e.g. spongistatin 1, spongistatin 2, spongistatin 3, spongistatin 4, spongistatin 5, spongistatin 6, spongistatin 7, spongistatin 8, and Spongistatin 9), cemadotin hydrochloride (i.e. LU-103793 and NSC-D-669356), epothilones (e.g. Epothilone A, Epothilone B, Epothilone C (i.e. desoxyepothilone A or dEpoA), epothilone D (i.e. KOS-862, dEpoB, and desoxyepothilone B), epothilone E, epothilone F, epothilone B N-oxide, epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), soblidotin (i.e. TZT-1027), vincristine sulfate, cryptophycin 52 (i.e. LY-355703), vitilevuamide, tubulysin A, canadensol, centaureidin (i.e. NSC-106969), Oncocidin A1 (i.e. BTO-956 and DIME), fijianolide B, laulimalide, narcosine (also known as NSC-5366), nascapine, hemiasterlin, vanadocene acetylacetonate, monsatrol, lnanocine (i.e. NSC-698666), eleutherobins (such as desmethyleleutherobin, desaetyleleutherobin, lsoeleutherobin A, and Z-Eleutherobin), caribaeoside, caribaeolin, halichondrin B, diazonamide A, taccalonolide A, diozostatin, (−)-phenylahistin (i.e. NSCL-96F037), myoseverin B, resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-Pseudomonas exotoxin conjugate, etc.), radioimmunotherapy (e.g., anti-CD20 monoclonal antibody conjugated to ¹¹¹In, ⁹⁰Y, or 1311 etc.), triptolide, homoharringtonine, dactinomycin, doxorubicin, epirubicin, topotecan, itraconazole, vindesine, cerivastatin, vincristine, deoxyadenosine, sertraline, pitavastatin, innotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

The singular terms “a”, “an”, and “the” include the plural reference unless the context clearly indicates otherwise.

The term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

### P Embodiments

Embodiment P1. A 6tdG-oligonucleotide comprising at least two 6-thio-2′-deoxyguanosine residues.

Embodiment P2. The 6tdG-oligonucleotide of Embodiment P1, comprising from three to about forty 6-thio-2′-deoxyguanosine residues.

Embodiment P3. The 6tdG-oligonucleotide of Embodiment P2, wherein at least three 6-thio-2′-deoxyguanosine residues are covalently bonded together via phosphate bonds.

Embodiment P4. The 6tdG-oligonucleotide of Embodiment P1, comprising from five to about forty 6-thio-2′-deoxyguanosine residues.

Embodiment P5. The 6tdG-oligonucleotide of Embodiment P4, wherein at least five 6-thio-2′-deoxyguanosine residues are covalently bonded together via phosphate bonds.

Embodiment P6. The 6tdG-oligonucleotide of any one of Embodiments P1 to P5, further comprising a nucleotide residue selected from the group consisting of an adenine residue, a deoxyadenine residue, a cytidine residue, a deoxycytidine residue, a uracil residue, or a deoxyuracil residue.

Embodiment P7. The 6tdG-oligonucleotide of any one of Embodiments P1 to P5, further comprising a nucleotide residue selected from the group consisting of an adenine residue, a deoxyadenine residue, a guanine residue, a deoxyguanine residue, a thymine residue, a deoxythymine residue, a cytidine residue, a deoxycytidine residue, a uracil residue, or a deoxyuracil residue.

Embodiment P8. The 6tdG-oligonucleotide of Embodiment P6 or P7, wherein at least one nucleotide residue is a modified nucleotide residue.

Embodiment P9. The 6tdG-oligonucleotide of Embodiment P8, wherein the modified nucleotide residue comprises a dideoxy modification, an inverted deoxybasic modification, a 2′-O-aminopropyl group, a 2′ constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, or a locked nucleic acid modification.

Embodiment P10. The 6tdG-oligonucleotide of any one of Embodiments P1 to P9, wherein the 6tdG-oligonucleotide comprises at least one phosphorothioated internucleotide linkage.

Embodiment P11. The 6tdG-oligonucleotide of any one of Embodiments P1 to P10, further comprising (i) from 1 to 12 adenosine residues; (ii) from 1 to 12 thymidine residues; (iii) a nucleic acid comprising from 1 to about 12 adenosine residues and from 1 to about 12 thymidine residues; or (iv) a combination of two or more of the foregoing.

Embodiment P12. The 6tdG-oligonucleotide of any one of Embodiments P1 to P11, further comprising a 6-thio-2′-deoxyguanosine mixmer.

Embodiment P13. The 6tdG-oligonucleotide of any one of Embodiments P1 to P12, wherein the 6tdG-oligonucleotide is a 6tdG-oligodeoxynucleotide.

Embodiment P14. The 6tdG-oligonucleotide of any one of Embodiments P1 to P13, wherein the 6tdG-oligonucleotide comprises a CpG motif.

Embodiment P15. The 6tdG-oligonucleotide of any one of Embodiments P1 to P12, wherein the 6tdG-oligonucleotide is a 6tdG-oligodeoxynucleotide comprising a CpG motif.

Embodiment P16. The 6tdG-oligonucleotide of Embodiment P15, wherein the 6tdG-oligonucleotide is selected from the group consisting of SEQ ID NOS:15-43.

Embodiment P17. The 6tdG-oligonucleotide of any one of Embodiments P1 to P16, further comprising a therapeutic moiety.

Embodiment P18. The 6tdG-oligonucleotide of Embodiment P17, wherein the therapeutic moiety is an antibody moiety.

Embodiment P19. The 6tdG-oligonucleotide of Embodiment P17, wherein the therapeutic moiety is a DNA aptamer or an RNA aptamer.

Embodiment P20. The 6tdG-oligonucleotide of Embodiment P17, wherein the therapeutic moiety is a STAT inhibitor moiety.

Embodiment P21. The 6tdG-oligonucleotide of any one of Embodiments P17 to P20, wherein the therapeutic moiety is at the 3′ end of the 6tdG-oligonucleotide or the 5′ end of the 6tdG-oligonucleotide.

Embodiment P22. The 6tdG-oligonucleotide of any one of Embodiments P1 to P21, further comprising a exonuclease resistant moiety.

Embodiment P23. The 6tdG-oligonucleotide of Embodiment P22, wherein the exonuclease resistant moiety is at the 3′ end of the 6tdG-oligonucleotide.

Embodiment P24. The 6tdG-oligonucleotide of Embodiment P15, wherein the 6tdG-oligodeoxynucleotide comprising the CpG motif and is a compound of Formula (I): 5′-R3-R1-L1-L2-L3-L4-L5-L6-R2-3′ (I), wherein: R1 is SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9; R2 is hydrogen, a phosphate group, an exonuclease resistant moiety, a therapeutic moiety, or a detectable moiety; R3 is absent, a therapeutic moiety, or a detectable moiety; L1, L2, L3, L4, L5, and L6 are each independently a bond, a guanosine residue, or a 6-thio-2′-deoxyguanosine residue, wherein at least two of L1, L2, L3, L4, L5, and L6 are a 6-thio-2′-deoxyguanosine residue.

Embodiment P25. The 6tdG-oligonucleotide of Embodiment P24, wherein L1, L2, L3, L4, L5, and L6 are each independently a guanosine residue or a 6-thio-2′-deoxyguanosine residue, wherein at least two of L1, L2, L3, L4, L5, and L6 are a 6-thio-2′-deoxyguanosine residue.

Embodiment P26. The 6tdG-oligonucleotide of Embodiment P25, wherein at least three of L1, L2, L3, L4, L5, and L6 is a 6-thio-2′-deoxyguanosine residue.

Embodiment P27. The 6tdG-oligonucleotide of Embodiment P26, wherein at least four of L1, L2, L3, L4, L5, and L6 is a 6-thio-2′-deoxyguanosine residue.

Embodiment P28. The 6tdG-oligonucleotide of Embodiment P27, wherein at least five of L1, L2, L3, L4, L5, and L6 is a 6-thio-2′-deoxyguanosine residue.

Embodiment P29. The 6tdG-oligonucleotide of Embodiment P28, wherein L1, L2, L3, L4, and L5 are a 6-thio-2′-deoxyguanosine residue, and L6 is a guanosine residue.

Embodiment P30. The 6tdG-oligonucleotide of Embodiment P28, wherein L1, L2, L3, L4, L5, and L6 are a 6-thio-2′-deoxyguanosine residue.

Embodiment P31. The 6tdG-oligonucleotide of any one of Embodiments P24 to P30, comprising at least one phosphorothioated internucleotide linker between L1 and L2; L2 and L3; L3 and L4; L4 and L5; or L5 and L6.

Embodiment P32. The 6tdG-oligonucleotide of any one of Embodiments P24 to P31, wherein R1 comprises at least one phosphorothioated internucleotide linker.

Embodiment P33. The 6tdG-oligonucleotide of Embodiment P32, wherein R1 is SEQ ID NO:10, SEQ ID NO:11, SEQ ID NO: 12, SEQ ID NO: 13, or SEQ ID NO:14.

Embodiment P34. The 6tdG-oligonucleotide of any one of Embodiments P24 to P33, wherein R2 is hydrogen.

Embodiment P35. The 6tdG-oligonucleotide of any one of Embodiments P24 to P33, wherein R2 is the exonuclease resistant moiety.

Embodiment P36. The 6tdG-oligonucleotide of Embodiment P22, P23, P24, or P35, wherein the exonuclease resistant moiety is —OP(═O)(OH)—O—(CH2)xOH, and x is an integer from 1 to 20.

Embodiment P37. The 6tdG-oligonucleotide of Embodiment P22, P23, P24, or P35, wherein the exonuclease resistant moiety comprises a modified nucleotide or a nucleic acid comprising a modified nucleotide.

Embodiment P38. The 6tdG-oligonucleotide of Embodiment P37, wherein the modified nucleotide comprises a dideoxy modification, an inverted deoxybasic modification, 2′-O-aminopropyl group, a 2′ constrained ethyl group, a 2′-fluoro group, a 2′-O-methyl group, 2′-deoxy-2′fluoro group, a 2′-O-methoxyethyl group, a 2′-O-allyl group, a 2′-O-propyl group, a 2′-O-pentyl group, or a locked nucleic acid modification.

Embodiment P39. The 6tdG-oligonucleotide of any one of Embodiments P22-P24 and P35-P38, wherein the exonuclease resistant moiety comprises a phosphorothioated internucleotide linker.

Embodiment P40. The 6tdG-oligonucleotide of any one of Embodiments P24 to P33, wherein R2 is the therapeutic moiety.

Embodiment P41. The 6tdG-oligonucleotide of any one of Embodiments P24 to P40, wherein R3 is the therapeutic moiety.

Embodiment P42. The 6tdG-oligonucleotide of Embodiment P24, P40, or P41, wherein the therapeutic moiety comprises a STAT inhibitor moiety.

Embodiment P43. The 6tdG-oligonucleotide of Embodiment P24, P40, or P41, wherein the therapeutic moiety comprises an antibody moiety.

Embodiment P44. The 6tdG-oligonucleotide of Embodiment P24, P40, or P41, wherein the therapeutic moiety comprises a DNA aptamer or an RNA aptamer.

Embodiment P45. The 6tdG-oligonucleotide of any one of Embodiments P24 to P40, wherein R3 is absent.

Embodiment P46. A pharmaceutical composition comprising the 6tdG-oligonucleotide of any one of Embodiments P1 to P45 and a pharmaceutically acceptable excipient.

Embodiment P47. A method of treating cancer in a patient in need thereof, the method comprising administering to the patient an effective amount of the 6tdG-oligonucleotide of any one of Embodiments P1 to P45 or the pharmaceutical composition of Embodiment P46.

Embodiment P48. A method of treating cancer in a patient in need thereof, the method comprising administering to the patient an effective amount of the 6tdG-oligonucleotide of any one of Embodiments P24 to P45, or a pharmaceutical composition comprising the 6tdG-oligonucleotide of any one of Embodiments P24 to P45 and a pharmaceutically acceptable excipient.

Embodiment P49. The method of Embodiment P47 or P48, further comprising administering to the patient an effective amount of a STAT inhibitor.

Embodiment P50. A method of treating cancer in a patient in need thereof, the method comprising administering to the patient an effective amount of a CpG oligodeoxynucleotide and an effective amount of 6-thio-2′-deoxyguanosine.

Embodiment P51. The method of Embodiment P50, further comprising administering to the patient an effective amount of a STAT inhibitor.

Embodiment P52. The method of any one of Embodiments P47 to P51, wherein the cancer expresses telomerase reverse transcriptase.

Embodiment P53. The method of any one of Embodiments P47 to P52, wherein the cancer is prostate cancer, colon cancer, non-small cell lung cancer, liver cancer, bladder cancer, pancreatic cancer, breast cancer, ovarian cancer, glioma, melanoma, head and neck cancer, renal cancer, leukemia, or lymphoma.

Embodiment P54. The method of any one of Embodiments P47 to P53, comprising intratumoral administration.

Embodiment P55. A pharmaceutical composition comprising a CpG oligonucleotide, 6-thio-2′-deoxyguanosine, and a pharmaceutically acceptable excipient.

Embodiment P56. The pharmaceutical composition of Embodiment P55, further comprising a STAT inhibitor.

### Example

It is understood that the example described herein is for illustrative purposes only and that various modifications or changes in light thereof will be suggested to persons skilled in the art and are to be included within the spirit and purview of this application and scope of the appended claims.

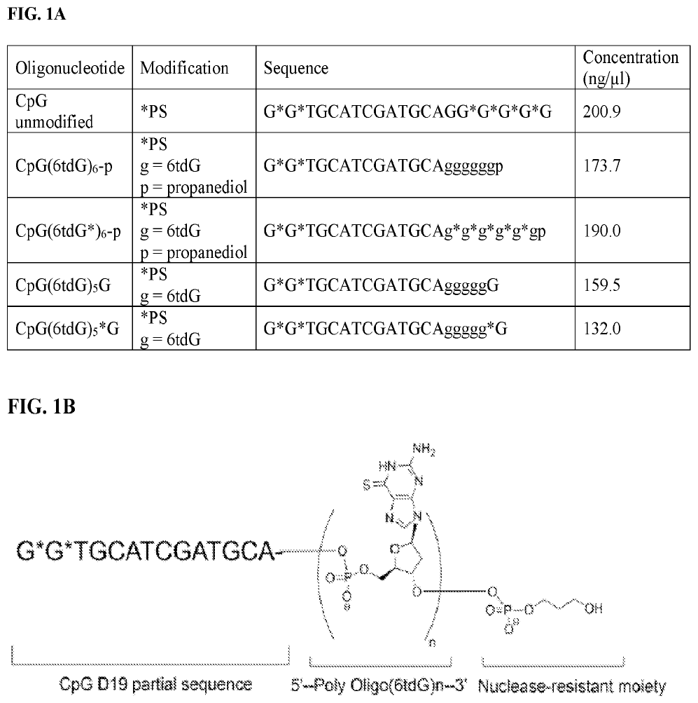

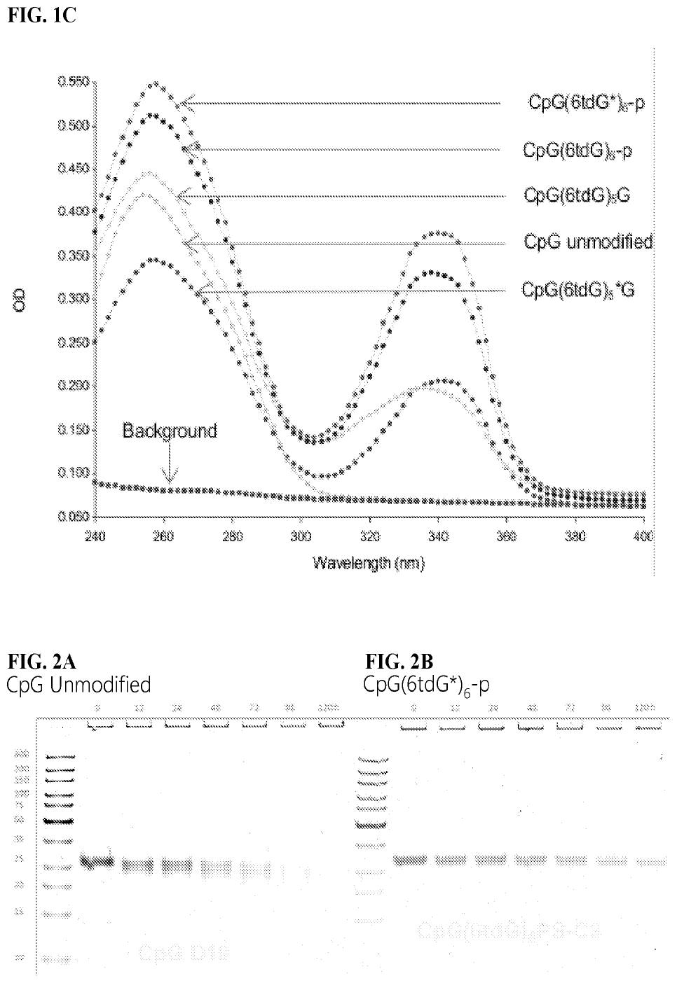

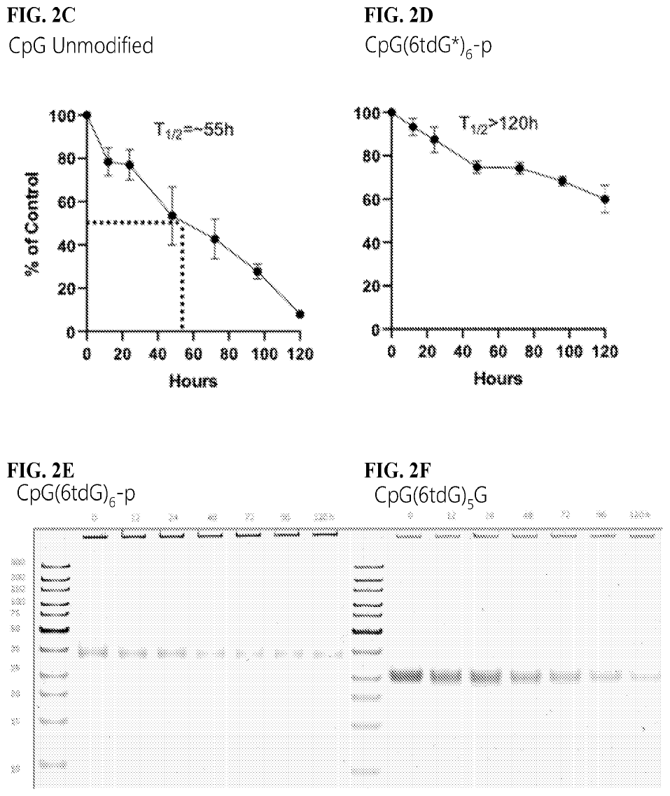

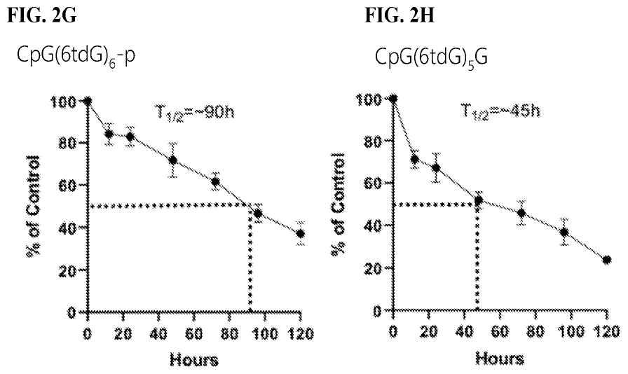

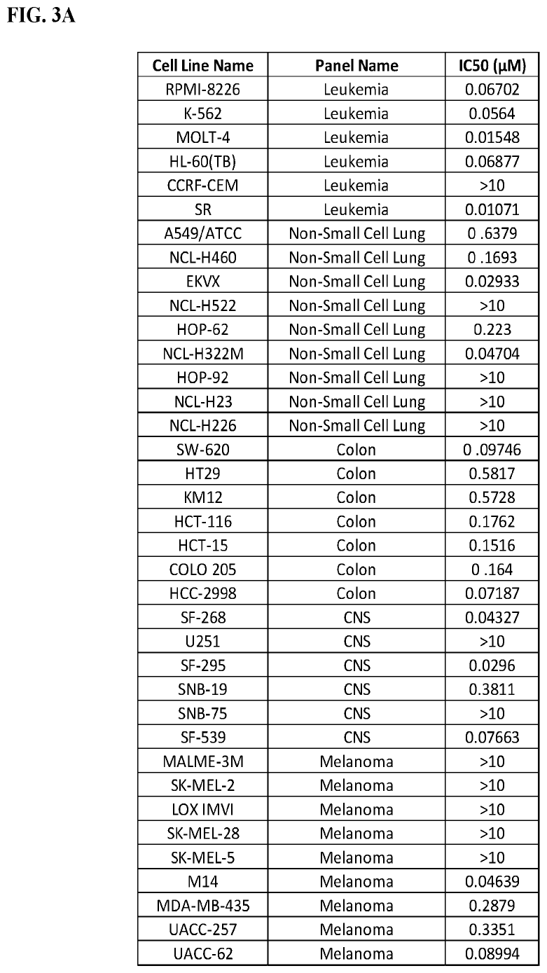

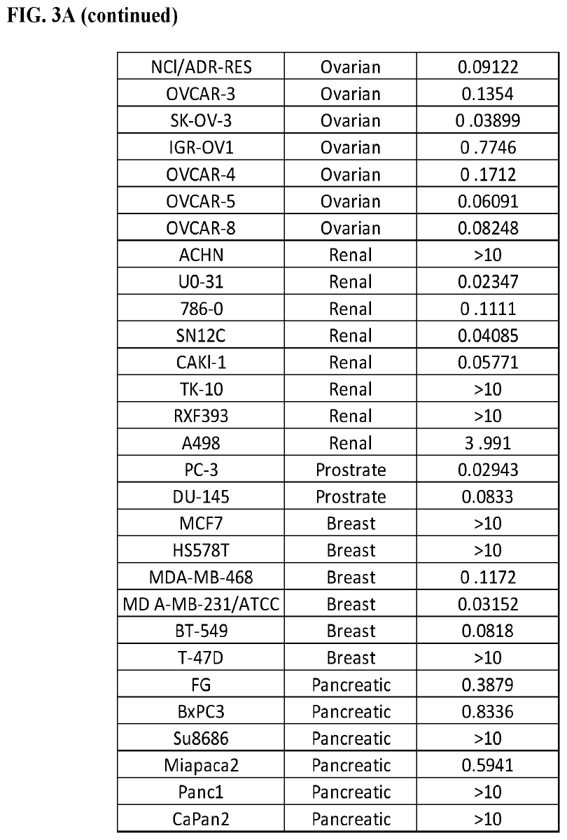

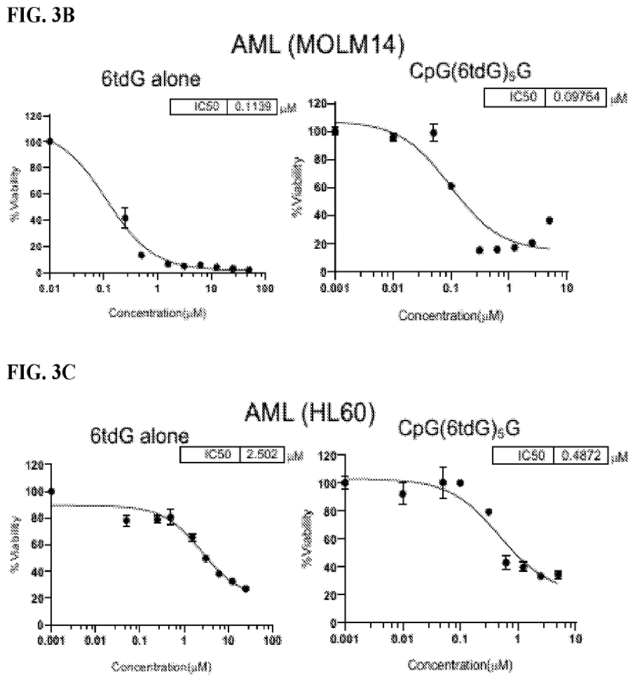

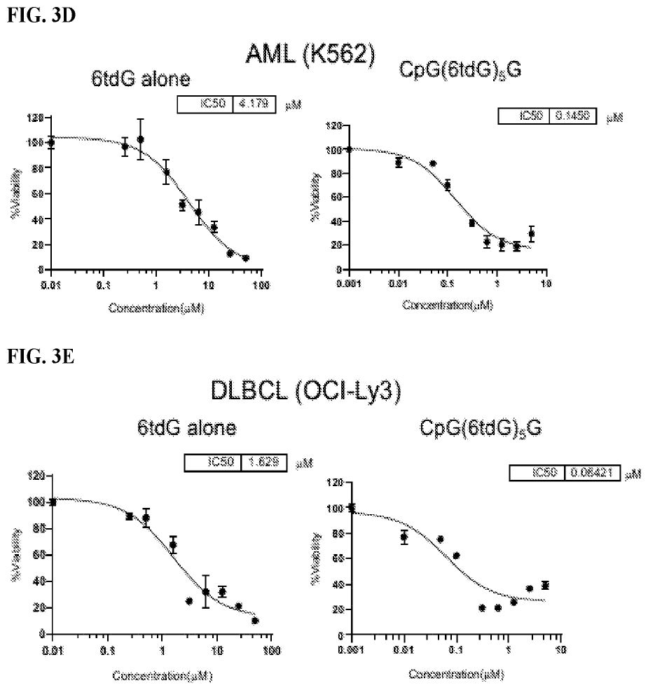

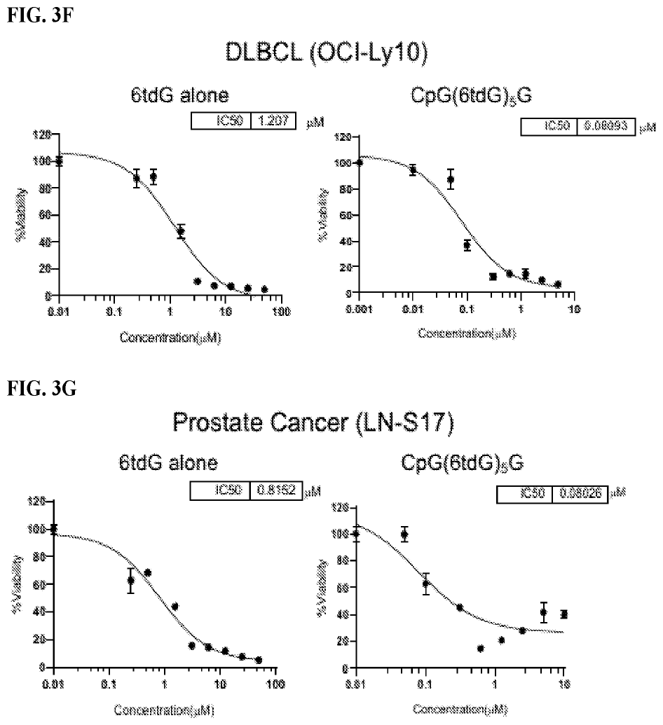

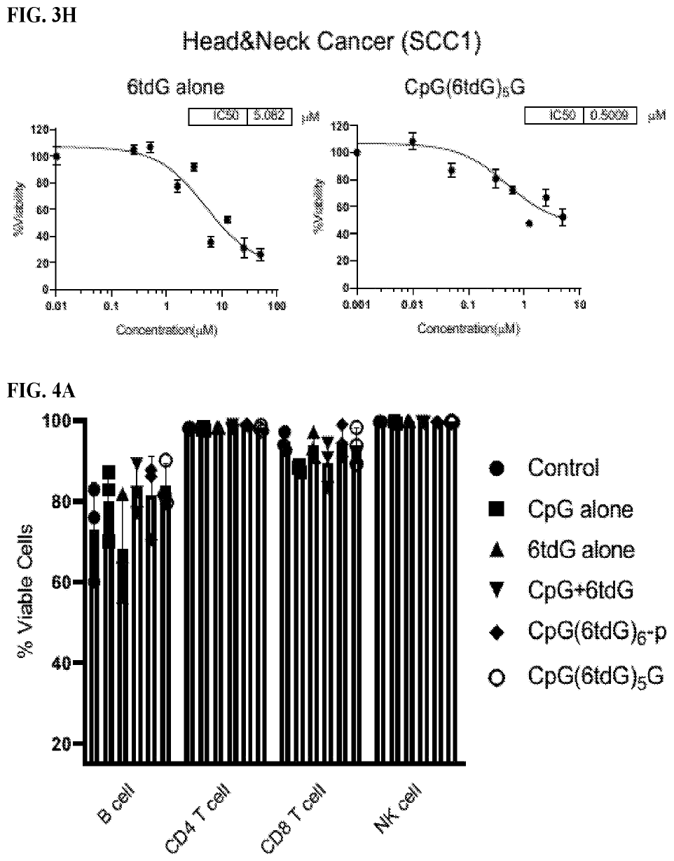

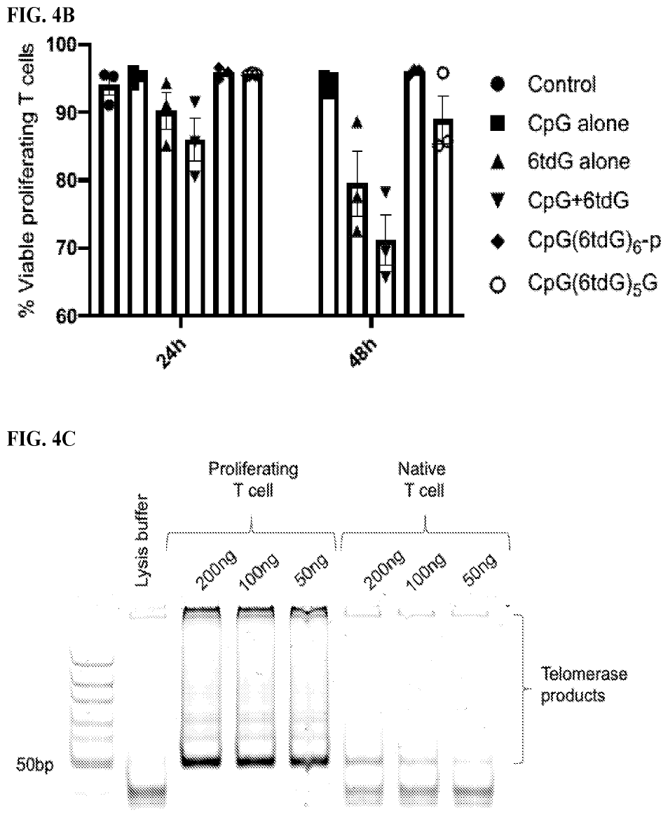

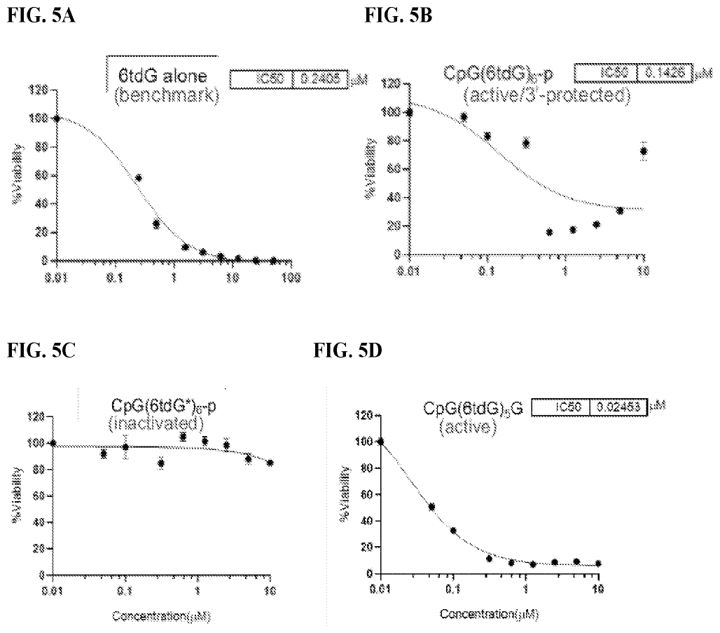

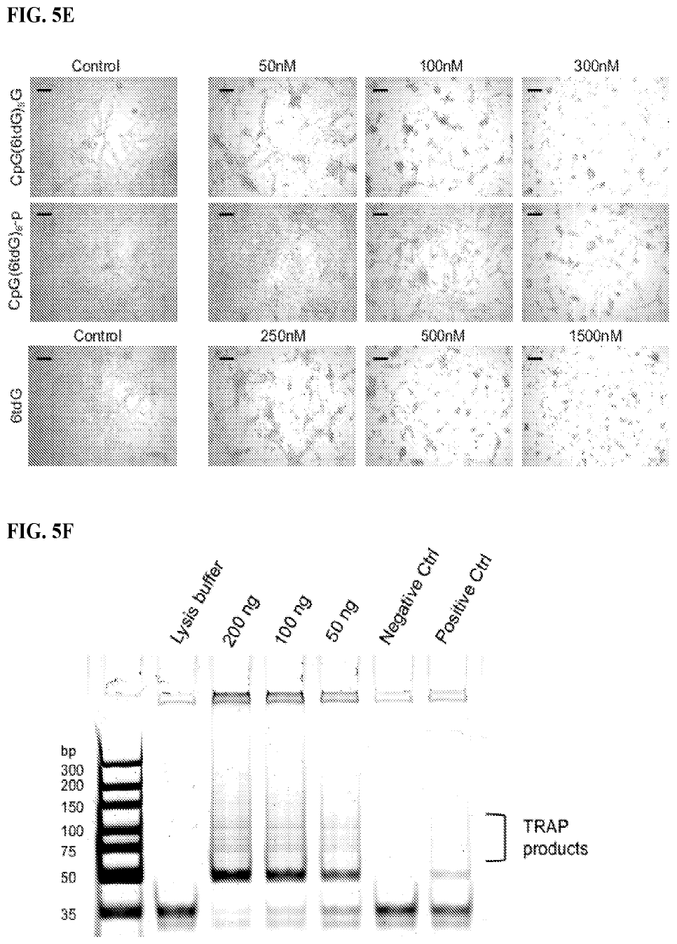

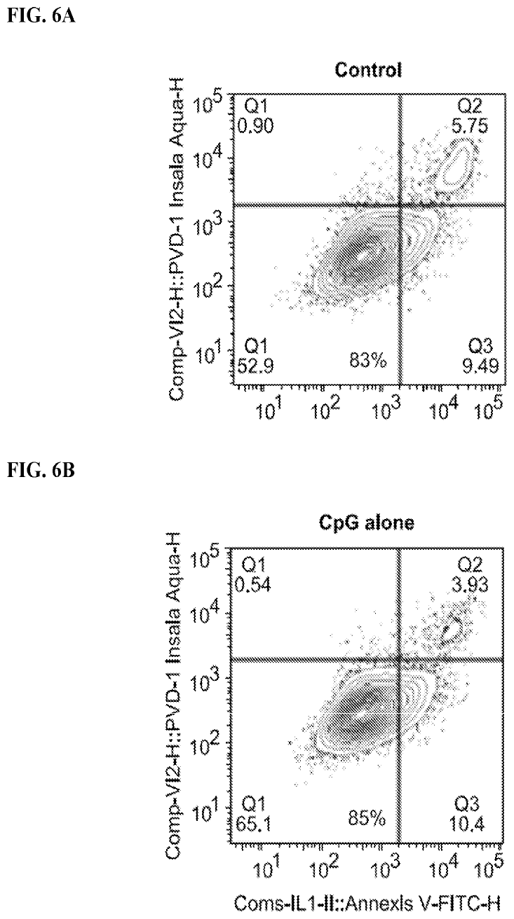

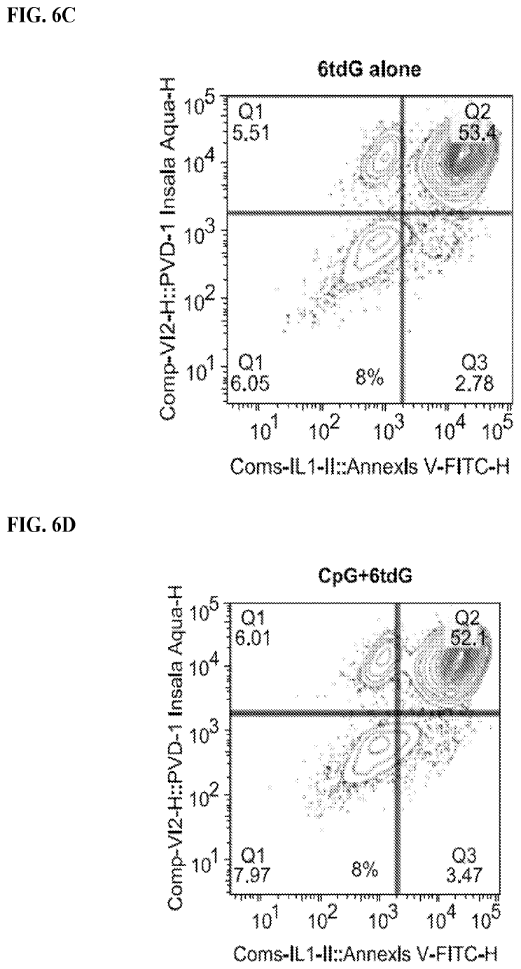

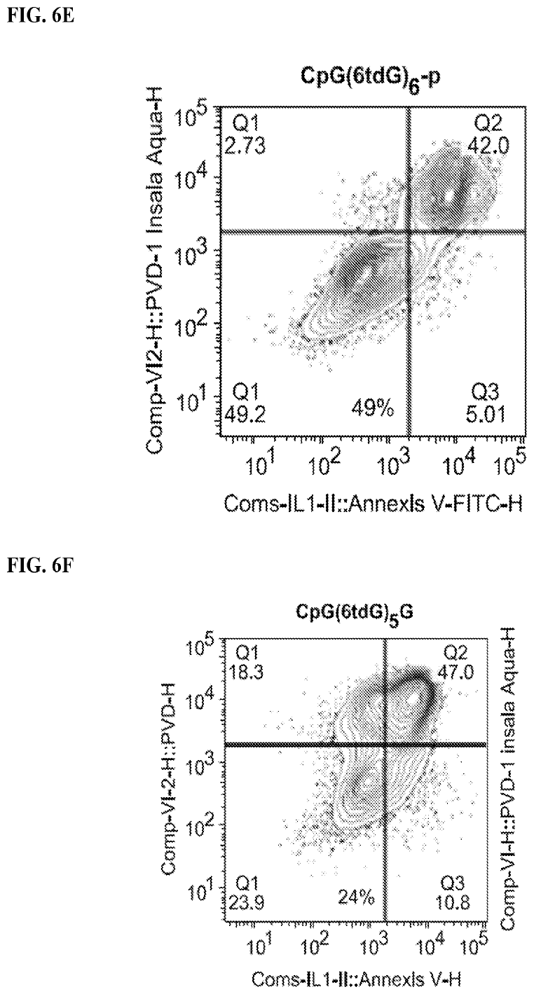

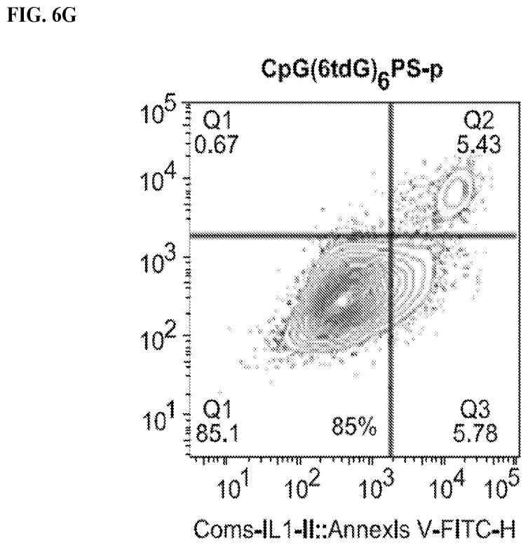

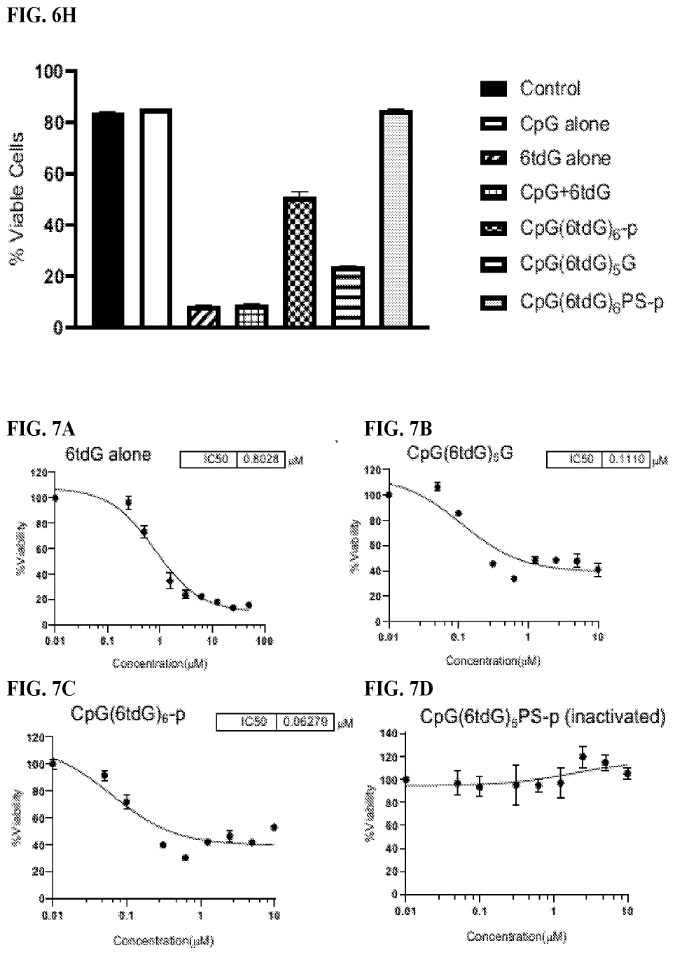

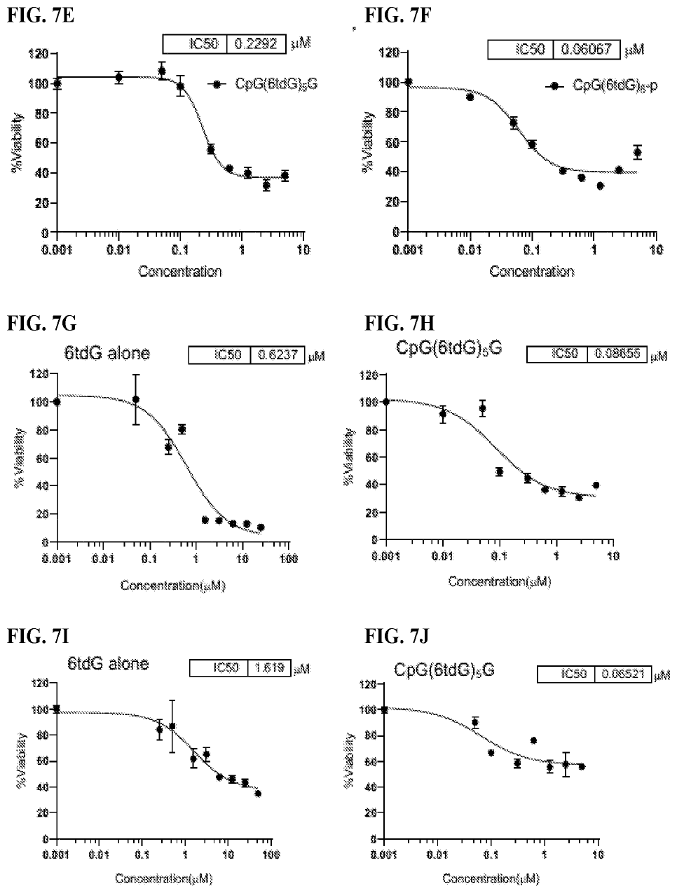

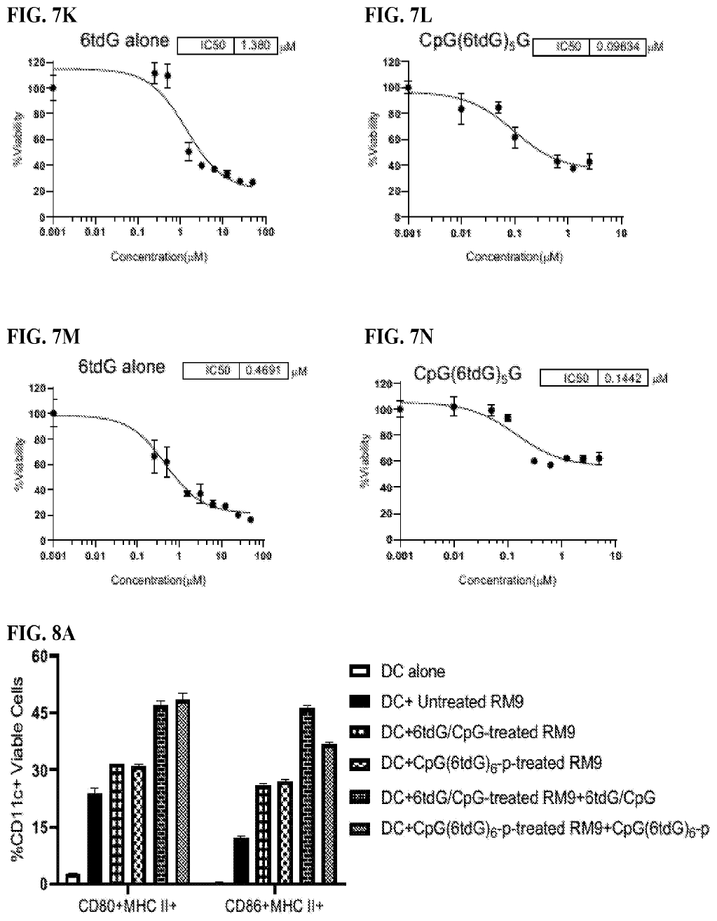

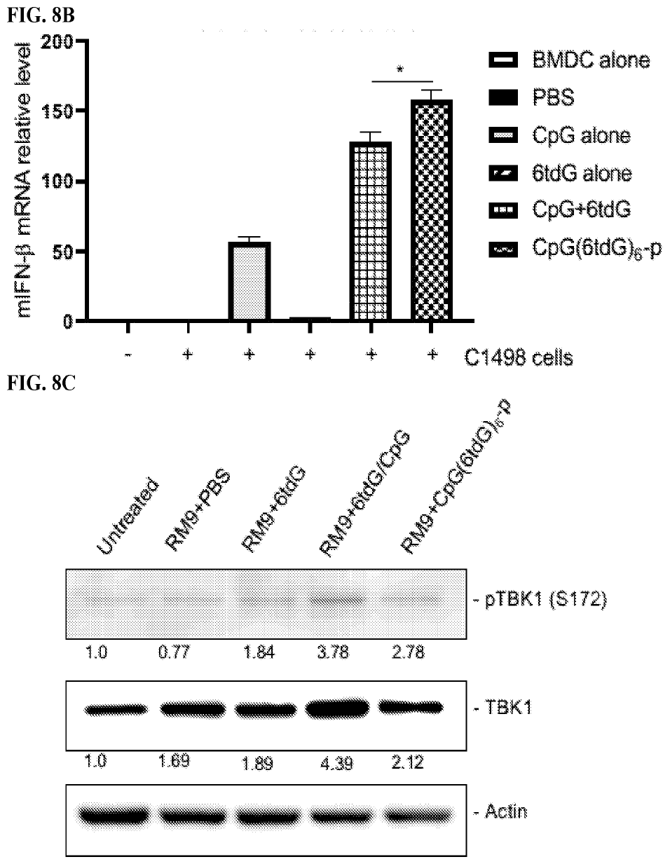

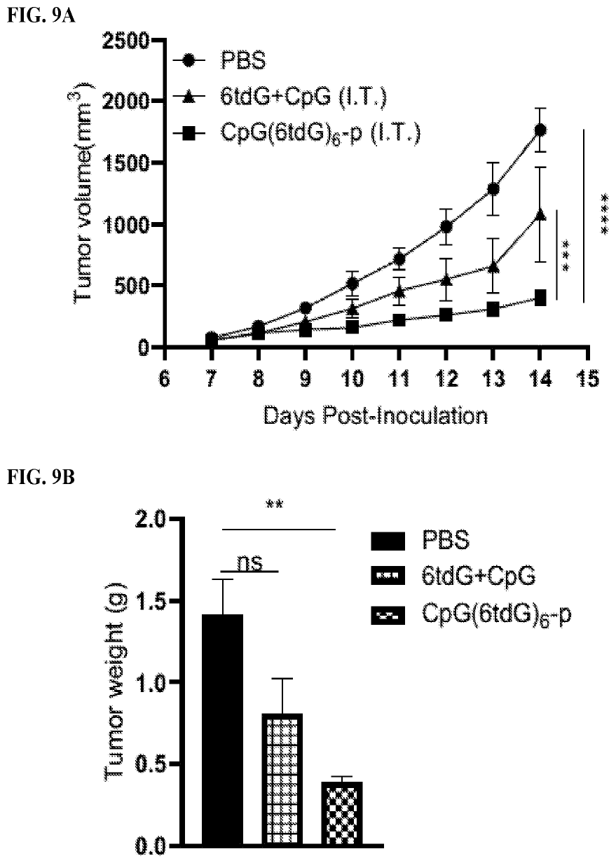

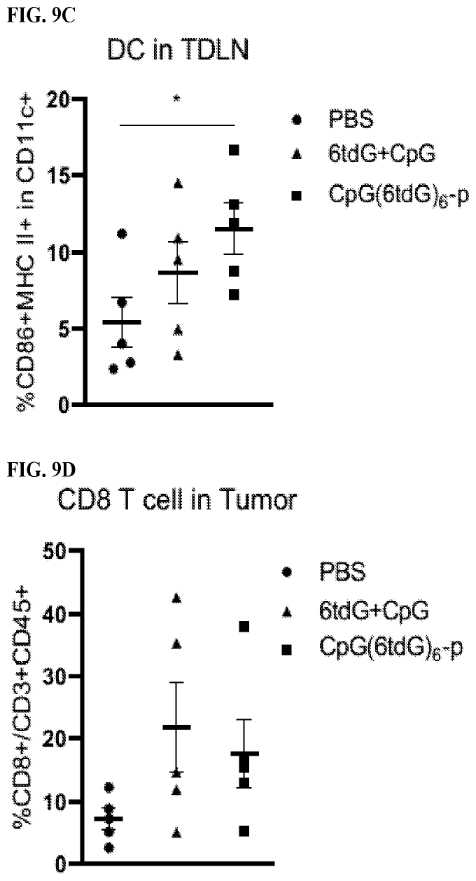

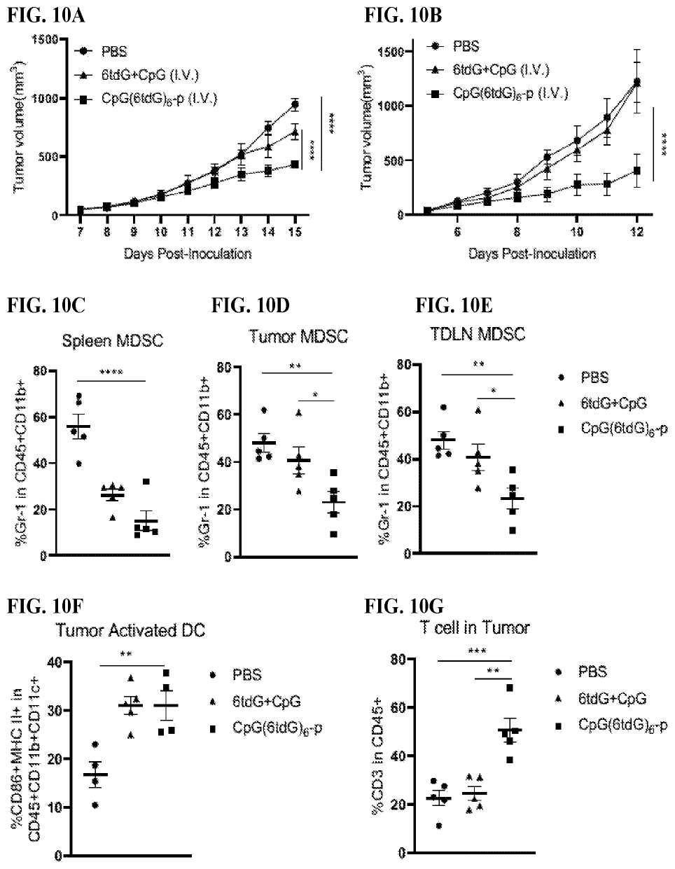

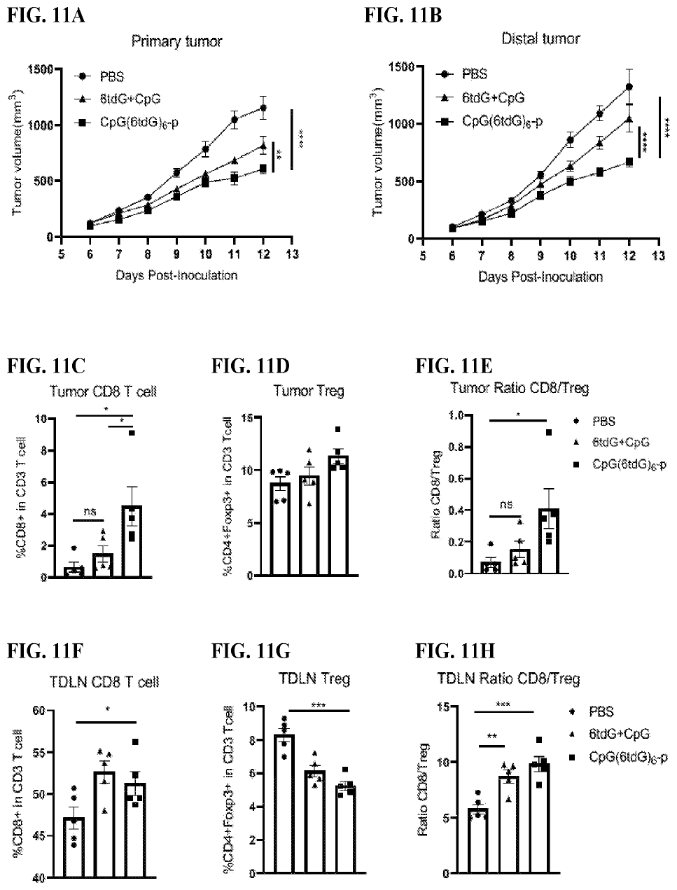

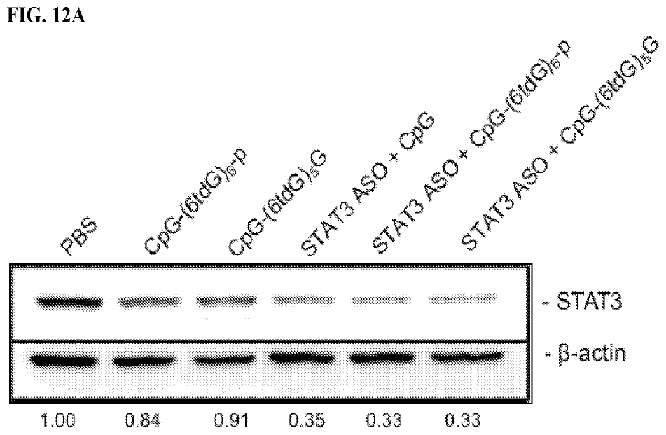

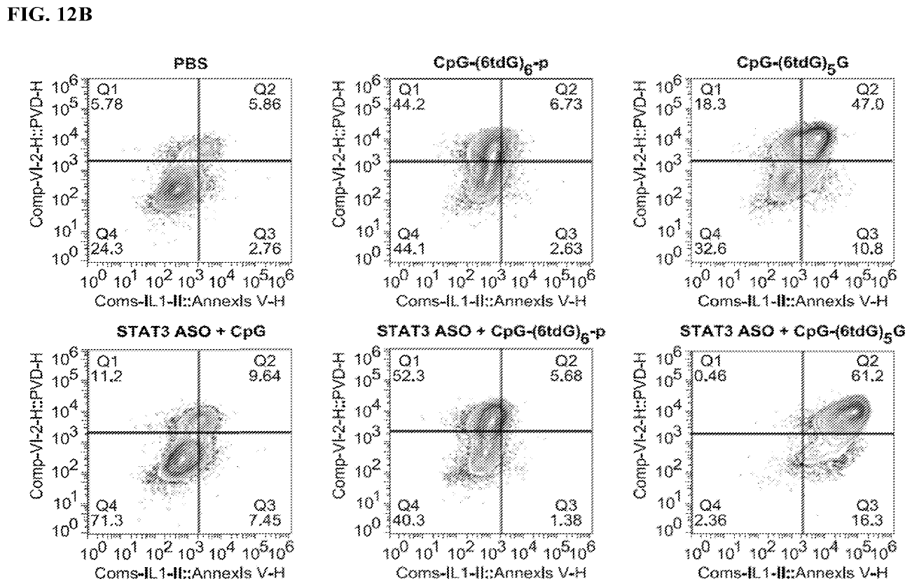

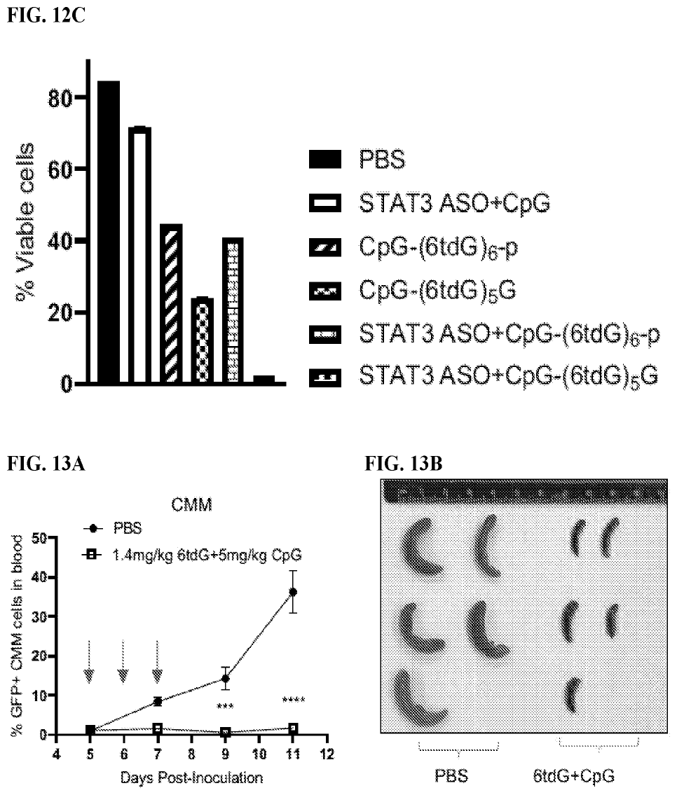

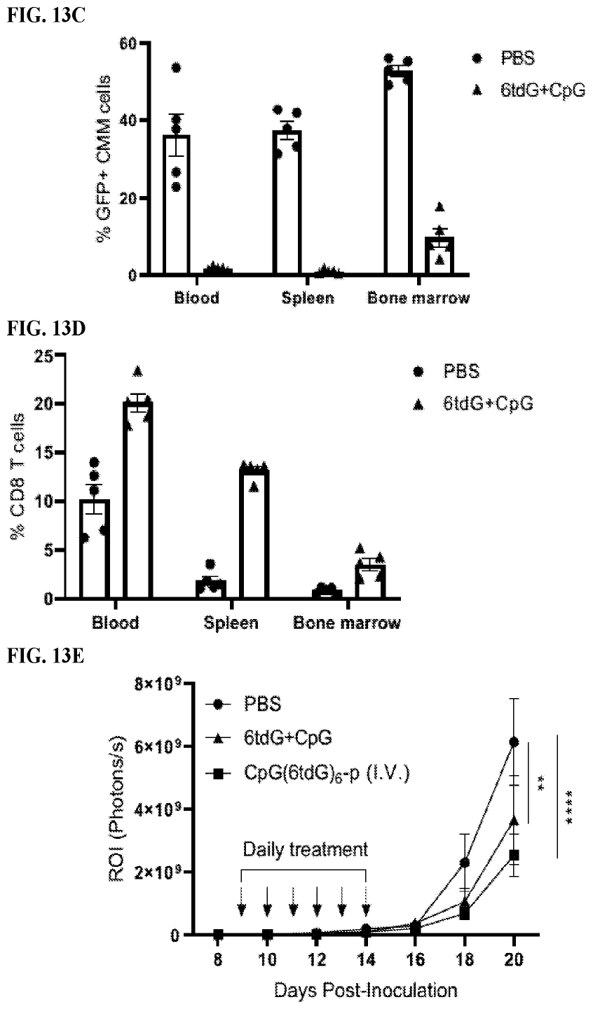

Our CpG-(6-thio-dG)ₙ or 6tdG oligodeoxynucleotides (ODN) consist of several (5-6) telomerase-blocking 6tdG nucleotide residues incorporated into the 3′ terminus of phosphorothioate oligodeoxynucleotide (ODN) with a single, unmethylated CpG motif (FIG. 1). The hybrid 6tdG ODN sequence (20 nts long) can be modified at 3′ terminus (e.g., FIG. 1B) for increased resistance to serum exonucleases, while still allowing for the intracellular release of the 6tdG nucleotides from the 3′ end (FIG. 2). Similar as the CpG ODNs, the 6tdG ODNs can target a variety of human and mouse cancer cells as well as myeloid cells, B lymphocytes but not T cells. In vitro, 6tdG ODN variants induce cell death in a variety of human (FIG. 3) and mouse cancer cells (FIGS. 5-7) with potency matching and sometimes exceeding the effect of 6tdG alone. In contrast, we did not find toxicity of 6tdGs to non-malignant immune cells or to activated T cells in contrast to 6tdG small molecule (FIG. 4) which can be ascribed to the disruption of telomere stability (FIG. 5). Importantly, the products of cancer cell death induced by 6tdG conjugates were found to have significantly higher immunostimulatory activity on mouse dendritic cells (DCs) than observed for CpG or 6tdG alone (FIG. 8). Our studies in two cancer models in mice confirmed that 6tdGs have shown strong antitumor effects. Local as well as systemic/intravenous administration of stabilized 6tdG variant (CpG(6tdG)₆-p) reduced growth of aggressive Ras Myc-driven RM9 prostate tumors and C1498 leukemia in mice (FIGS. 9-13). These effects are likely driven by both immune-mediated and direct anticancer effects as indicated by significant inhibition of prostate tumor growth in immunocompetent (FIGS. 9A and 10) as well as in immunodeficient mice (FIG. 9B). The induction of antitumor immune responses was evidenced by the abscopal effect of 6tdG treatment (FIG. 11) with the increased percentages of tumor-infiltrating CD8 T cells (FIGS. 11C-11D). Importantly, therapeutic effects of 6tdG ODNs were further enhanced by an inhibition of STAT3 signaling, which is known to reduce cancer cell survival and immune evasion (FIG. 12).

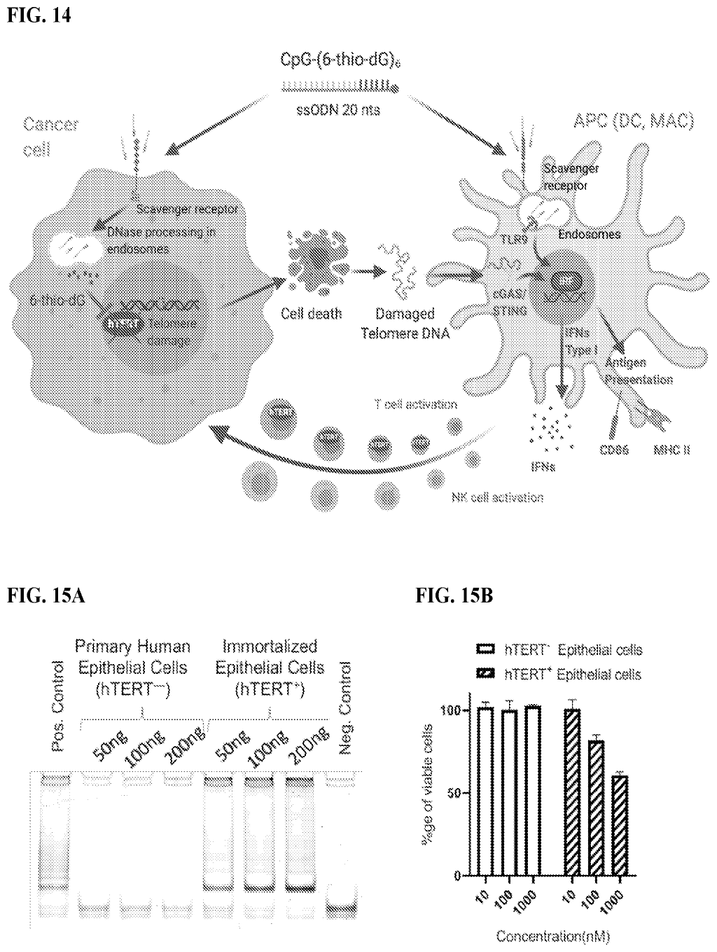

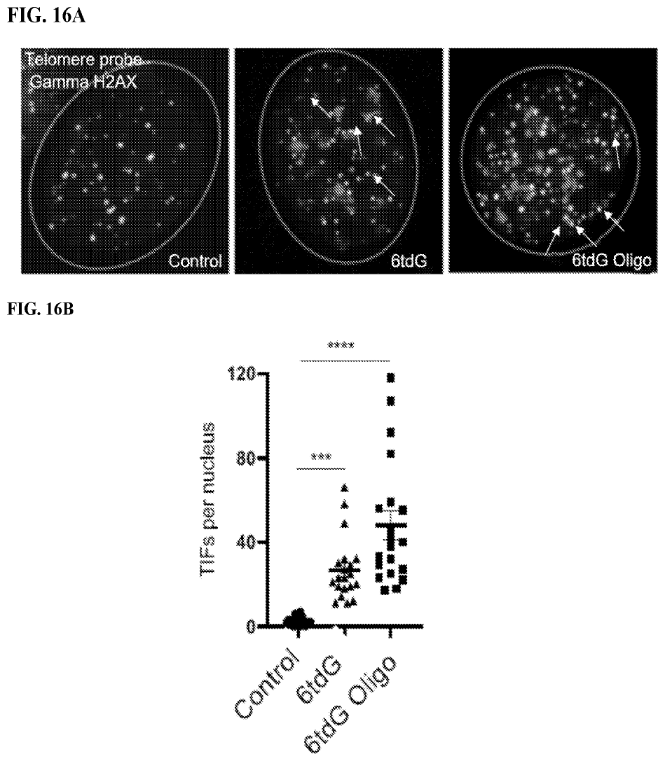

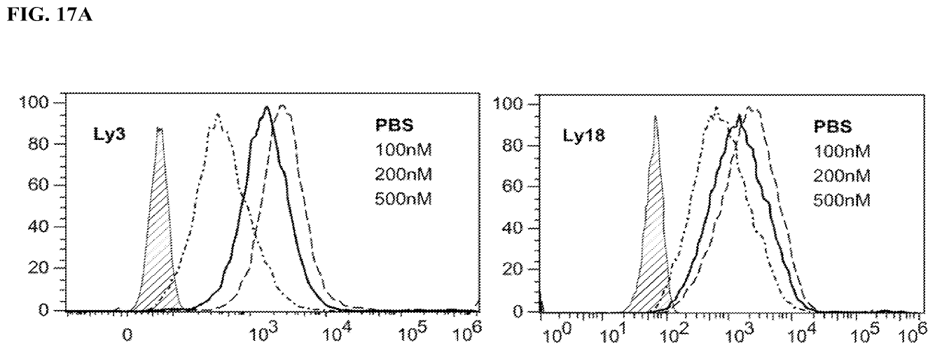

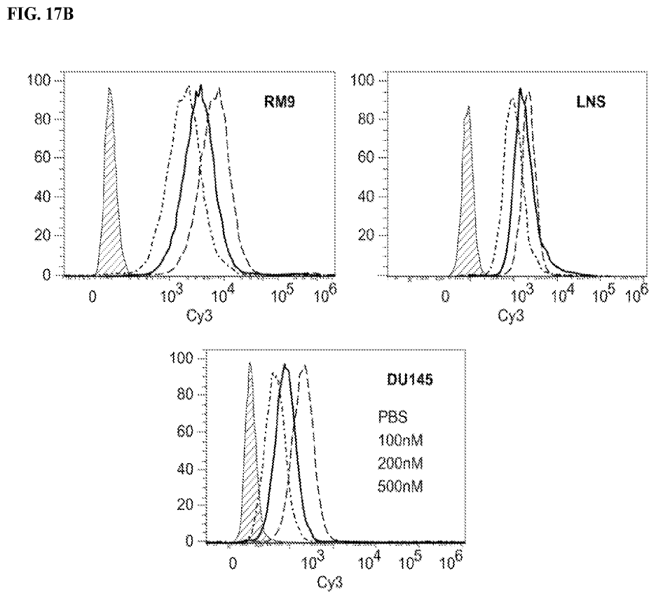

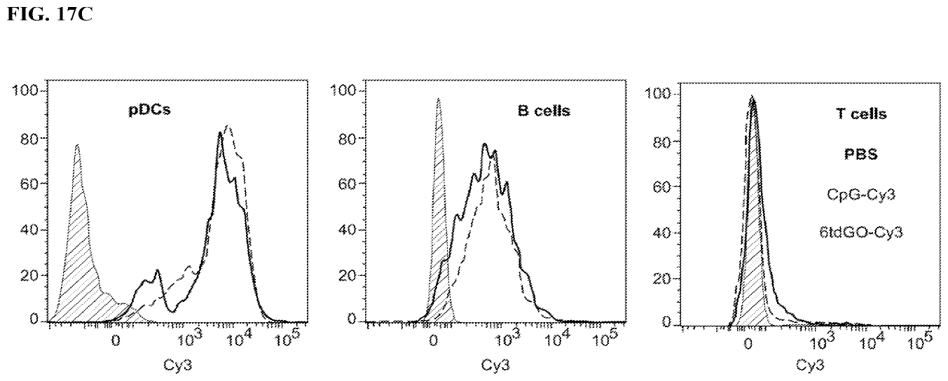

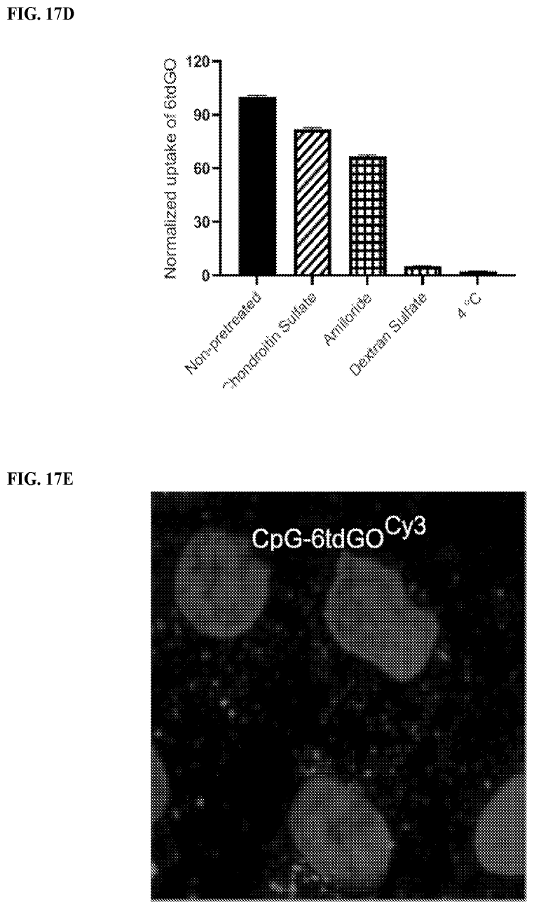

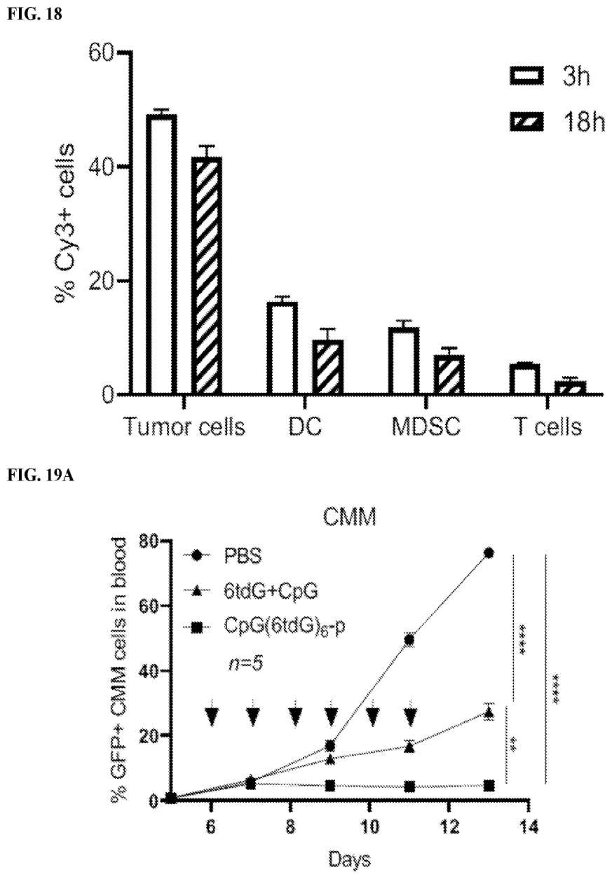

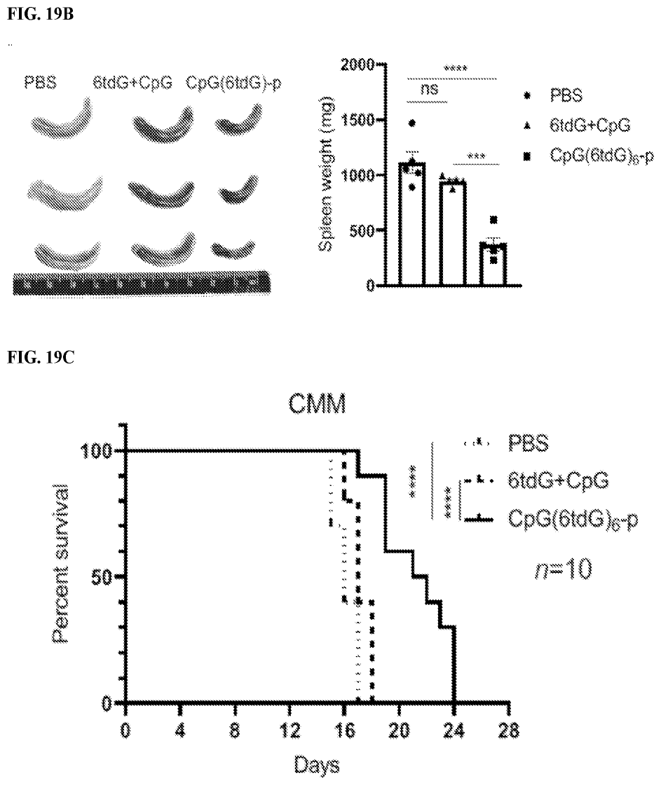

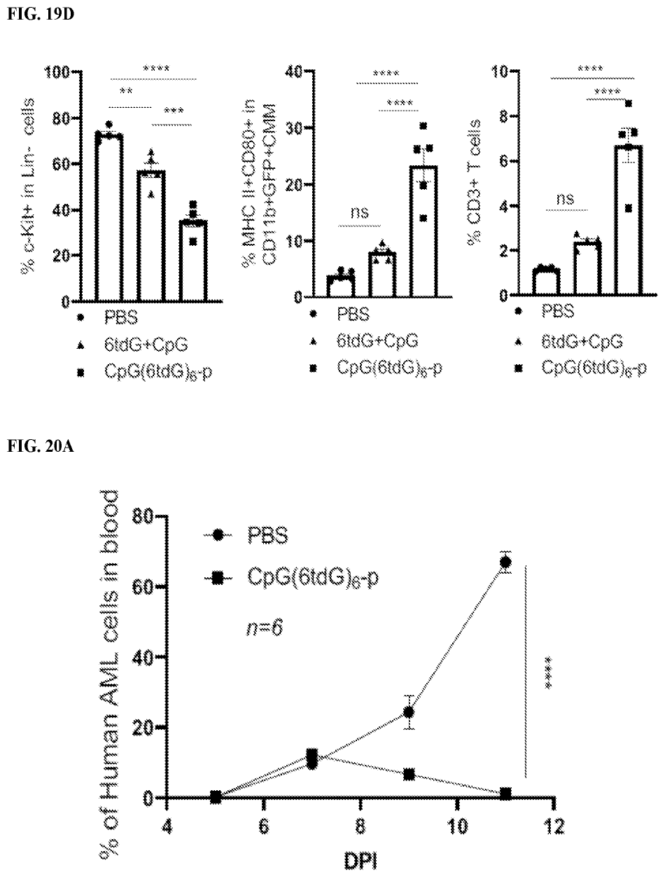

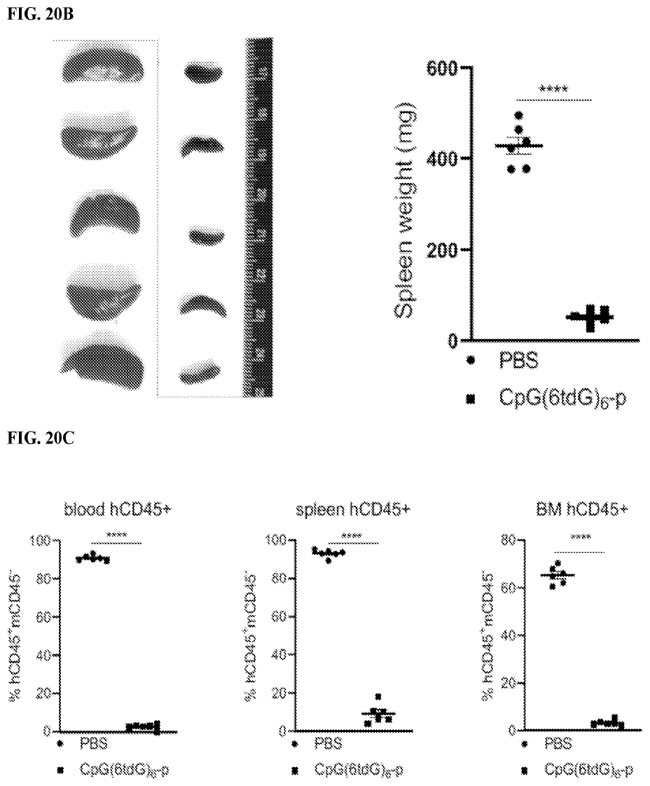

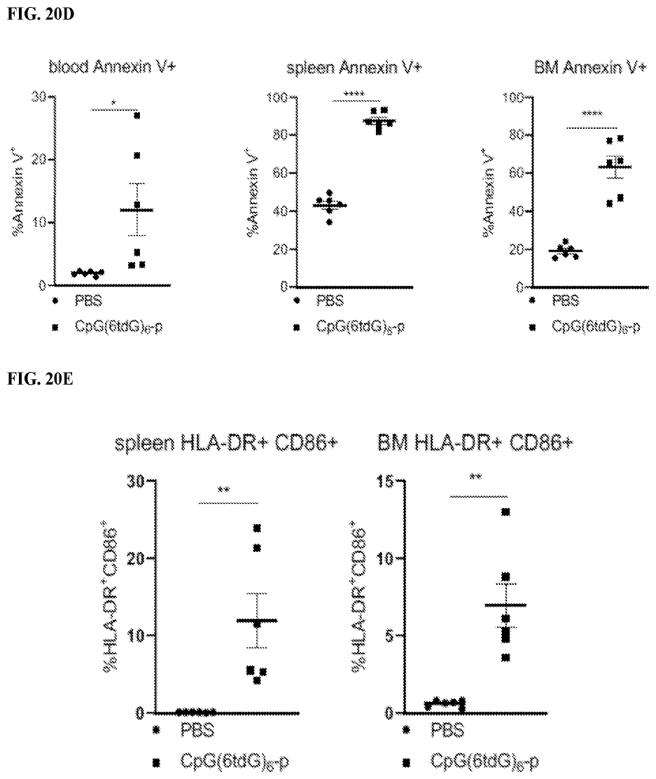

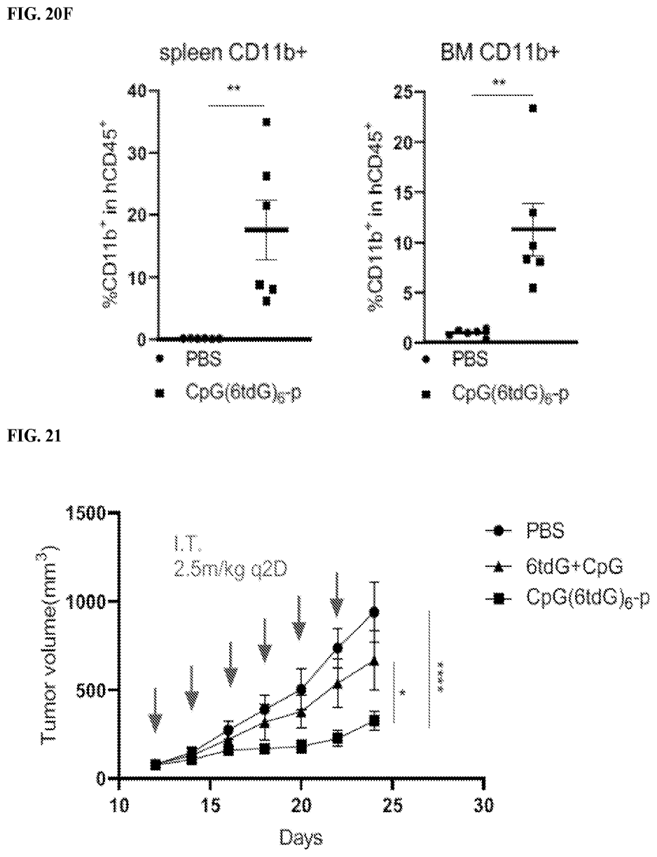

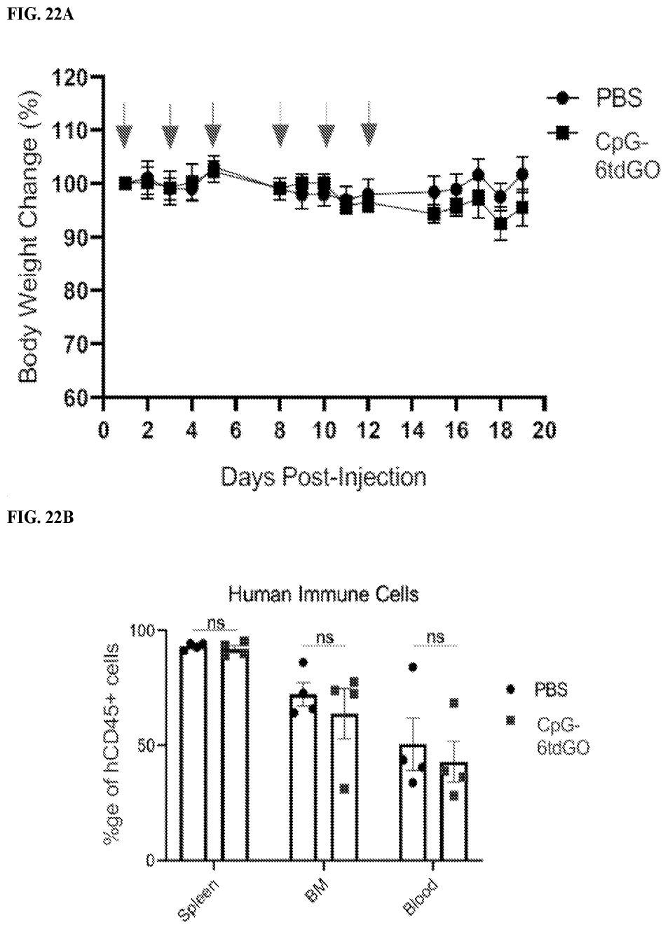

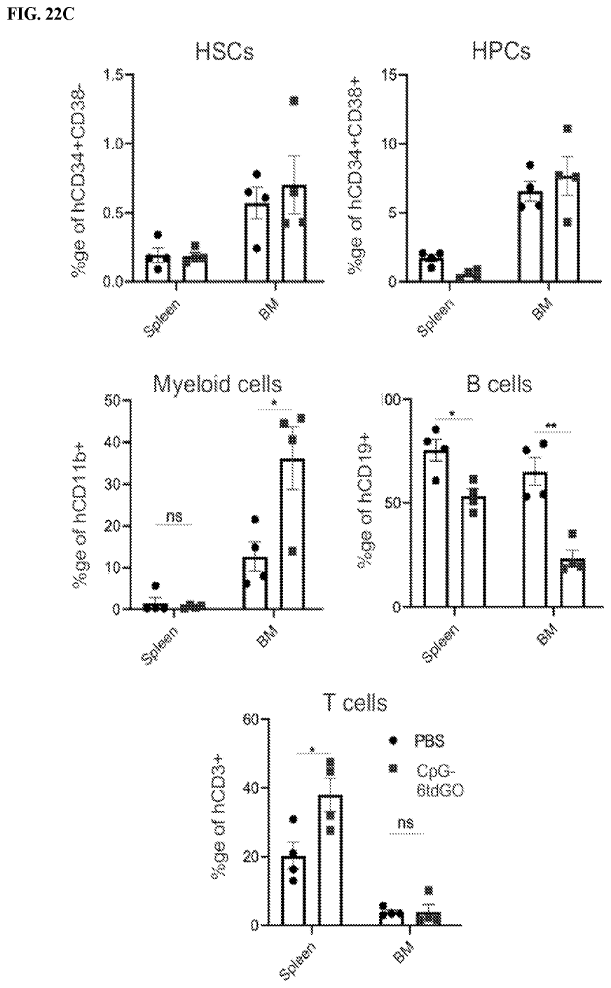

The putative mechanism of 6tdG ODN antitumor effects is two-pronged. In addition to the direct cytotoxicity to cancer cells, 6tdGs likely activate antigen-presenting cells (APCs) using at least two molecular mechanisms (FIG. 14). As typical for standard CpG ODNs, 6tdGs can activate immunostimulatory TLR9 signaling in specialized immune cells (DCs and macrophages). In addition, the telomere-associated DNA released from dying cancer cells can trigger cGAS/STING activation in immune cells. The activation of immune signaling by endosomal TLR9 and cytosolic STING should synergistically augment the antigen-presentation and production of immune mediators, such as type I interferons (IFNα/β), which are critical for antitumor immune responses. Our initial results suggest that therapeutic effect of 6tdG ODN can be a double whammy resulting from the direct cytotoxicity to cancer cells followed by an induction of potent antitumor immunity.

Thus, the putative mechanism of 6tdG ODN antitumor effects is two-pronged. In addition to the direct cytotoxicity to cancer cells, 6tdGs likely activate antigen-presenting cells (APCs) using at least two molecular mechanisms. As typical for standard CpG ODNs, 6tdGs can activate immunostimulatory TLR9 signaling in specialized immune cells (DCs and macrophages). In addition, the telomere-associated DNA released from dying cancer cells can trigger cGAS/STING activation in immune cells. The activation of immune signaling by endosomal TLR9 and cytosolic STING should synergistically augment the antigen-presentation and production of immune mediators, such as type I interferons (IFNa/b), which are critical for antitumor immune responses. Our initial results indicate that therapeutic effect of 6tdG ODN can be a double whammy resulting from the direct cytotoxicity to cancer cells followed by an induction of potent antitumor immunity.

### Methods

Chemical Synthesis. Synthesis of various CpG-(6-thio-dG)n conjugates was performed at 10 micromole scale (OligoPilot10 Synthesizer/GE), using the solid phase synthesis and standard phosphoramidite chemistry. DNA amidite were purchased from ThermoFisher, 3′-Spacer C3 CPG solid support from Glen Research and 6-Thio-dG-CE Phosphoramidite from ChemGenes was used for the introduction of the 6-thio deoxy Guanosine into the oligonucleotide.

Synthesis Parameters. Amidite Concentration: 200 mM. Activator: 0.5M ETT. Coupling: standard coupling time for deoxy-phosphoramidites. Oxidation: use 0.02 M Iodine in Pyridine-Water, (9:1) for the oxidation. Deprotection: deprotect with 1.0 M 1,8-Diazabicyclo(5.4.0)undec-7-ene (DBU) in anhydrous acetonitrile at Room Temperature for 5 h to remove the cyanoethyl protection. Wash the support two times with acetonitrile after the removal of cyanoethyls is completed. Final deprotection was completed with 50 mM NaSH in concentrated NH4OH at Room Temperature (e.g., about 20° C. to about 22° C.) for 36 h. After the deprotection was completed the reaction mixture was evaporated to dryness under the reduced pressure. Oligonucleotide was then purified on the GE Purifier using ion-paired purification on the PRP-1 resin from Hamilton using 10 mM TBAA buffers at pH 7.5. The product was analyzed on the analytical 15% PAGE and by an analytical HPLC on the Agilent 1200 HPLC system using HAA buffers.

1-56. (canceled)

57. A 6tdG-oligonucleotide comprising at least two 6-thio-2′-deoxyguanosine residues.

58. The 6tdG-oligonucleotide of claim 57, comprising from three to about forty 6-thio-2′-deoxyguanosine residues.

59. The 6tdG-oligonucleotide of claim 58, wherein at least three 6-thio-2′-deoxyguanosine residues are covalently bonded together via phosphate bonds.

60. The 6tdG-oligonucleotide of claim 59, wherein at least five 6-thio-2′-deoxyguanosine residues are covalently bonded together via phosphate bonds.

61. The 6tdG-oligonucleotide of claim 57, further comprising a nucleotide residue selected from the group consisting of an adenine residue, a deoxyadenine residue, a guanine residue, a deoxyguanine residue, a thymine residue, a deoxythymine residue, a cytidine residue, a deoxycytidine residue, a uracil residue, or a deoxyuracil residue.

62. The 6tdG-oligonucleotide of claim 61, wherein at least one nucleotide residue is a modified nucleotide residue; wherein the modified nucleotide residue comprises a dideoxy modification, an inverted deoxybasic modification, a 2′-O-aminopropyl group, a 2′ constrained ethyl group, a 2′-fluoro group, a 2′-O-methyl group, 2′-deoxy-2′-fluoro group, a 2′-O-methoxyethyl group, a 2′-O-allyl group, a 2′-O-propyl group, a 2′-O-pentyl group, or a locked nucleic acid modification.

63. The 6tdG-oligonucleotide of claim 57, wherein the 6tdG-oligonucleotide comprises at least one phosphorothioated internucleotide linkage.

64. The 6tdG-oligonucleotide of claim 57, further comprising a 6-thio-2′-deoxyguanosine mixmer.

65. The 6tdG-oligonucleotide of claim 57, wherein the 6tdG-oligonucleotide is a 6tdG-oligodeoxynucleotide comprising a CpG motif.

66. The 6tdG-oligonucleotide of claim 65, wherein the 6tdG-oligonucleotide is any one of SEQ ID NOS:15-43.

67. The 6tdG-oligonucleotide of claim 65, wherein the 6tdG-oligodeoxynucleotide is a compound of Formula (I):
(I)
5′-R³-R¹-L¹-L²-L³-L⁴-L⁵-L⁶-R²,
wherein:
R¹ is SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14;
R² is hydrogen, a phosphate group, an exonuclease resistant moiety, a therapeutic moiety, or a detectable moiety;
R³ is absent, a therapeutic moiety, or a detectable moiety;
L¹, L², L³, L⁴, L⁵, and L⁶ are each independently a bond, a guanosine residue, or a 6-thio-2′-deoxyguanosine residue, wherein at least two of L¹, L², L³, L⁴, L⁵, and L⁶ are a 6-thio-2′-deoxyguanosine residue.

68. The 6tdG-oligonucleotide of claim 67, wherein:
(a) L¹, L², L³, L⁴, and L⁵ are a 6-thio-2′-deoxyguanosine residue, and L⁶ is a guanosine residue, or
(b) L¹, L², L³, L⁴, L⁵, and L⁶ are a 6-thio-2′-deoxyguanosine residue.

69. The 6tdG-oligonucleotide of claim 67, comprising at least one phosphorothioated internucleotide linker between L¹ and L²; L² and L³; L³ and L⁴; L⁴ and L⁵; or L⁵ and L⁶.

70. The 6tdG-oligonucleotide of claim 67, wherein R² is hydrogen.

71. The 6tdG-oligonucleotide of claim 67, wherein R² is the exonuclease resistant moiety, wherein the exonuclease resistant moiety is: (i) —OP(═O)(OH)—O—(CH₂)ₓOH, and x is an integer from 1 to 20, (ii) a modified nucleotide, or (iii) a nucleic acid comprising a modified nucleotide.

72. The 6tdG-oligonucleotide of claim 71, wherein the modified nucleotide comprises a dideoxy modification, an inverted deoxybasic modification, 2′-O-aminopropyl group, a 2′ constrained ethyl group, a 2′-fluoro group, a 2′-O-methyl group, 2′-deoxy-2′-fluoro group, a 2′-O-methoxyethyl group, a 2′-O-allyl group, a 2′-O-propyl group, a 2′-O-pentyl group, or a locked nucleic acid modification.

73. The 6tdG-oligonucleotide of claim 67, wherein R³ is absent.

74. A pharmaceutical composition comprising the 6tdG-oligonucleotide of claim 57 and a pharmaceutically acceptable excipient.

75. A pharmaceutical composition comprising a CpG oligonucleotide, 6-thio-2′-deoxyguanosine, and a pharmaceutically acceptable excipient.

76. A method of treating cancer in a patient in need thereof, the method comprising administering to the patient:
(i) an effective amount of the 6tdG-oligonucleotide of claim 57, or
(ii) an effective amount of a CpG oligodeoxynucleotide and an effective amount of 6-thio-2′-deoxyguanosine.
